(19) United States
(12) Patent Application Publication
(10) Pub. No.: US 2022/0241208 A1
(43) Pub. Date: Aug. 4, 2022

(54) PHARMACEUTICAL COMPOSITIONS FOR THE TREATMENT OF HBV

(71) Applicant: ASSEMBLY BIOSCIENCES, INC., San Francisco, CA (US)

(72) Inventors: Lee D. ARNOLD, Mount Sinai, NY (US); George Koan WONG, Belle Mead, NJ (US); Kirk HENNE, Belmont, CA (US); James Francis HULVAT, Cincinnati, OH (US); Sanjay KONAGURTHU, Bend, OR (US); Ian Scott MCINTOSH, Bend, OR (US); Matthew David WESSEL, Sisters, OR (US); Thomas REYNOLDS, Bend, OR (US); Richard J. COLONNO, San Francisco, CA (US); Uri A. LOPATIN, San Francisco, CA (US)

(21) Appl. No.: 17/613,670

(22) PCT Filed: May 22, 2020
(86) PCT No.: PCT/US2020/034367

§ 371 Date: Nov. 23, 2021

Related U.S. Application Data
(60) Provisional application No. 62/852,705, filed on May 24, 2019.
(60) Provisional application No. 63/020,927, filed on May 6, 2020.

(51) Int. Cl. A61K 9/20; A61K 9/10; A61K 31/554; A61K 47/32; A61K 47/38; A61K 9/16; A61K 47/26; A61K 47/02; A61K 47/14; A61K 45/06; A61P 31/20

(52) U.S. Cl. CPC A61K 9/2027; A61K 9/10; A61K 31/554; A61K 47/32; A61K 47/38; A61K 9/16; A61P 31/20; A61K 47/02; A61K 47/14; A61K 9/2095; A61K 9/2054; A61K 45/06; A61K 47/26

(57) ABSTRACT

The present disclosure provides, in part, pharmaceutical compositions that comprise a spray dried dispersion which contains the disclosed compound, and optionally, pharmaceutical excipients. The pharmaceutical compositions of the disclosure may be used in the treatment of Hepatitis B (HBV).

### CROSS REFERENCE TO RELATED APPLICATIONS

This application claims the benefit of, and priority to, U.S. provisional patent application Ser. No. 62/852,705, filed May 24, 2019, and U.S. provisional patent application Ser. No. 63/020,927 filed May 6, 2020, each of which are hereby incorporated by reference herein in their entirety.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleotide analogs (e.g., entecavir) that suppress the virus while the patient remains on treatment, but do not eliminate the infection, even after many years of treatment. Once a patient starts taking nucleotide analogs, most must continue taking them or risk the possibility of a life-threatening immune response due to viral rebound. Further, nucleotide therapy may lead to the emergence of antiviral drug resistance.

The only FDA approved alternative to nucleotide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients is likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

### SUMMARY

The present disclosure provides pharmaceutical compositions and methods of preparing pharmaceutical compositions for the treatment of Hepatitis B (HBV). In one aspect, the present disclosure provides pharmaceutical compositions comprising: a solid dispersion of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, in a polymer.

In some embodiments, the solid dispersion includes about 10 wt % to about 50 wt % of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, and about 40 wt % to about 90 wt % of the polymer. In some embodiments, the solid dispersion includes about 25 wt % to about 50 wt % of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, and about 50 wt % to about 75 wt % of the polymer. In some embodiments, the solid dispersion includes about 20 wt % of 11-oxo-N-((2-(trifluoromethyl)thiazol-5-yl)methyl)-10,11 dihydrodibenzo[b,f][1,4]thiazepine-8-carboxamide 5,5-dioxide, or a pharmaceutically acceptable salt thereof, and about 80 wt % of the polymer.

In some embodiments, the polymer includes a group capable of hydrogen bonding, such as a carboxylate group, and a hydrophobic group or region, such as an aromatic group. In some embodiments, the polymer is capable of interactions between 11-oxo-N-((2-(trifluoromethyl) thiazol-5-yl)methyl)-10,11-dihydrodibenzo[b,f][1,4]thiazepine-8-carboxamide 5,5-dioxide and the polymer (e.g., hydrogen bonding interactions, hydrophobic interactions, or a combination thereof).

In some embodiments, the polymer is a methacrylate polymer or a cellulosic polymer. In some embodiments, the polymer is a poly(methacrylic acid-co-methyl methacrylate), hypromellose acetate succinate, or hydroxypropyl methylcellulose phthalate polymer. In some embodiments, the polymer is a poly(methacrylic acid-co-methyl methacrylate) polymer. In some embodiments, the polymer is a hypromellose acetate succinate polymer. In some embodiments, the polymer is a hydroxypropyl methylcellulose phthalate polymer.

In some embodiments, the pharmaceutical composition is a spray-dried solid dispersion. In some embodiments, the solid dispersion is amorphous or substantially amorphous. In some embodiments, the amorphous or substantially amorphous solid dispersion has a single T_{g}. In some embodiments, the amorphous or substantially amorphous solid dispersion is stable for at least four weeks.

In some embodiments, the pharmaceutical composition further comprises a pharmaceutically acceptable excipient. Suitable excipients include fillers, sweeteners, diluents, binders, lubricants, disintegrants, and glidants, or a combination thereof. In some embodiments, the pharmaceutical composition further comprises microcrystalline cellulose, mannitol, talc, croscarmellose sodium, magnesium stearate, or sodium lauryl sulfate, or a combination thereof. In some embodiments, the pharmaceutical composition further comprises a colorant, fragrance, or flavoring agent.

In some embodiments, the solid dispersion further comprises a pharmaceutically acceptable excipient. Suitable excipients include fillers, sweeteners, diluents, binders, lubricants, disintegrants, and glidants, or a combination thereof. In some embodiments, the solid dispersion further comprises microcrystalline cellulose, mannitol, talc, croscarmellose sodium, magnesium stearate, or sodium lauryl sulfate, or any combination thereof. In some embodiments, the solid dispersion further comprises a colorant, fragrance, or flavoring agent.

In some embodiments, the pharmaceutical composition is in a dose form, such as a granule, a pellet, a tablet, a particle, or a mini-tablet. In some embodiments, the dose form includes about 75 mg to about 125 mg of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof. In some embodiments, the dose form includes about 300 mg of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof.

In another aspect, the present disclosure provides a method of treating Hepatitis B (HBV) in a patient in need thereof, comprising: administering to the patient a therapeutically effective amount of a pharmaceutical composition as described herein.

In another aspect, the present disclosure provides methods for preparing the pharmaceutical compositions described herein. The method generally comprises: combining 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, with a polymer in a solvent to form a mixture and drying the mixture, thereby forming a solid dispersion. The solid dispersion can be optionally combined with at least one excipient. In some embodiments, drying the mixture can include spray drying the mixture.

In some embodiments, the solvent used in the methods includes water. In some embodiments, the solvent includes an organic solvent. In some embodiments, the solvent includes acetone and water.

In some embodiments, methods of preparing a pharmaceutical composition can further include compressing the pharmaceutical composition into a tablet.

Provided herein, in part, are methods of treating hepatitis B in a subject in need thereof, the method comprising administering daily to the subject for example, about 300 mg of a dosage amount as disclosed herein, of a compound represented by:

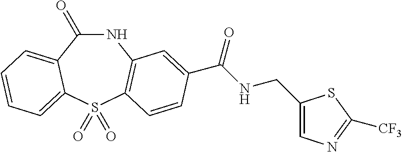 [SMILES: O=C(NCc1cnc(C(F)(F)F)s1)c1ccc2c(c1)NC(=O)c1ccccc1S2(=O)=O]

(also referred to as Compound 1); and administering to the subject a therapeutically effective amount of a nucleos(t)ide inhibitor, such as one selected from the group consisting of entecavir, tenofovir and tenofovir alafenamide fumarate. In some embodiments, the subject is virologically suppressed and HBeAg negative before administering the compound. In other aspects, the subject is virologically suppressed and HBeAg positive before administering the compound. In other embodiments, the subject is treatment naïve and HBeAg positive before administering the compound.

In some other embodiments, a contemplated subject is virologically suppressed for at least 6 months and/or has previously been administered a nucleos(t)ide inhibitor alone, for example, a subject which has been previously administered a nucleos(t))ide inhibitor alone for at least 2 months.

In another embodiment, a subject may not have been previously administered nucleos(t)ide inhibitor.

Contemplated subjects may have detectable levels of hepatitis B viral DNA prior to administration. For example, a subject may be positive for the hepatitis B e-antigen (HBeAg). Such HBeAg positive subjects may, after about 24 weeks, 36 weeks or more (such as time interval as disclosed herein) of daily administration as described in the disclosed methods, have sustained HBeAg loss of <0.11 PEI units/mL.

Also contemplated herein are methods of treating HBeAg negative patients prior to daily administration as disclosed.

In certain aspects, a disclosed method may include daily administration of compound 1 and administration of a therapeutically effective amount of a nucleos(t)ide inhibitor, for at least 12 weeks, 24 weeks, 28 weeks, 32 weeks, 40 weeks, 44 weeks or more.

In some aspects, after 24 weeks or more of daily administration as disclosed herein the subject has a reduction of HBeAg and/or HBsAg, for example the subject may have a loss or stable reduction of HBsAg to <100 IU/mL, and/or the subject may have sustained viral suppression (e.g. below the limits of detection=20 IU/mL).

Alternatively or additionally, a subject may have a reduction in HBV DNA or HBV RNA, for example, the HBV DNA reduction may be below the detectable limit—for example as detected using a PCR-assay. In some embodiments, the HBV RNA is below the limit of detection.

Such disclosed methods, e.g., after about 24 weeks or more of daily administration as disclosed herein, may result in subject with greater than or about equal to a 0.5 log₁₀ decline in HBeAg, e.g., a disclosed method may reduce hepatitis B virus to below detection levels in the subject.

In other aspects, disclosed herein are methods of treating hepatitis B in a subject who is virologically suppressed and HBeAg negative, wherein the subject is administered daily about 300 mg of a compound represented by:

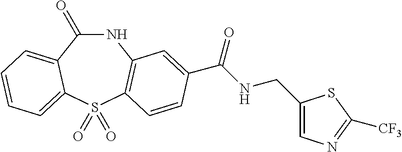 [SMILES: O=C(NCc1cnc(C(F)(F)F)s1)c1ccc2c(c1)NC(=O)c1ccccc1S2(=O)=O]

and
administering to the subject a therapeutically effective amount of a nucleos(t)ide inhibitor such as entecavir, tenofovir or tenofovir alafenamide fumarate; and the subject was virologically suppressed and HBeAg negative before administration of the compound; and wherein if after the 76^{th} week of administering the compound and the nucleos(t)ide the subject has a hepatitis B viral DNA concentration of less than 20 IU/mL and a HBeAg concentration of less than or equal to 5 IU/mL for at least six months prior to the 76^{th} week of administering the compound, administration of the compound and the nucleos(t)ide inhibitor is stopped. In the other aspects, method further comprises monitoring the subject for up to three years for hepatitis B viral DNA concentration and HBeAg concentration after the 76^{th} week of administering the compound. In some aspects, the nucleos(t)ide inhibitor is entecavir. In some aspects, the compound is in a solid dosage form. In some other aspects, the compound is in a solid dispersion. In other aspects, the solid dispersion further comprises a polymer. In some embodiments, the solid dispersion further comprises an excipient. In some other embodiments, the compound is administered to the subject is in a solid spray dispersion as disclosed herein. In some other embodiments, the compound is administered in a pharmaceutical composition as disclosed herein.

Some embodiments disclosed here are methods of treating hepatitis B in a subject who is virologically suppressed and HBeAg positive the method comprising administering daily to the subject about 300 mg of a compound represented by:

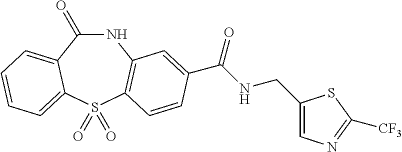 [SMILES: O=C(NCc1cnc(C(F)(F)F)s1)c1ccc2c(c1)NC(=O)c1ccccc1S2(=O)=O]

and
administering to the subject a therapeutically effective amount of a nucleos(t)ide inhibitor such as entecavir, tenofovir or tenofovir alafenamide fumarate; and the subject was virologically suppressed and HBeAg positive before administration of the compound;
wherein if, after 76 weeks of administering the compound and the nucleosi(t)e the subject has a hepatitis B viral DNA concentration of less than 20 IU/mL and a HBeAg concentration of less than or equal to 5 IU/mL for at least six months prior to the 76^{th} week, the administration of the compound and the nucleos(t)ide inhibitor is stopped; or, after 76 weeks of administering the compound,
the subject has a hepatitis B viral DNA concentration of greater than or equal to 20 IU/mL or a HBeAg concentration of greater than 5 IU/mL during the six months prior to the 76^{th} week of administering the compound, administration of the compound is stopped and administration of the nucleos(t)ide inhibitor is continued. In some aspects, the method further comprises monitoring the subject for up to three years after the 76^{th} week of administering the compound for hepatitis B viral DNA concentration and HBeAg concentration if the subject has a hepatitis B viral DNA concentration of less than 20 IU/mL and a HBeAg concentration of less than or equal to 5 IU/mL for at least six months prior to the 76^{th} week of administering the compound. In other embodiments, the method further comprises monitoring the subject for up to twelve weeks after the 76^{th} week of administering the compound for hepatitis B viral DNA concentration and HBeAg concentration if the subject has a hepatitis B viral DNA concentration of greater than or equal to 20 IU/mL or a HBeAg concentration of greater than 5 IU/mL during the six months prior to the 76^{th} week of administering the compound. In some aspects, the nucleos(t)ide inhibitor is entecavir. In further embodiments, the compound is a solid dosage form. In further aspects, the compound is in a solid dispersion. In some aspects, the solid dispersion further comprises a polymer. In some other aspects, the solid dispersion further comprises an excipient. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein.

Some embodiments described herein are methods of treating hepatitis B in subject who is treatment naïve and HBeAg positive, the method comprising: administering daily to the subject about 300 mg of Compound 1 and administering to the subject a therapeutically effective amount of a nucleos(t)ide inhibitor selected from the group consisting of entecavir, tenofovir and tenofovir alafenamide fumarate; and the subject was treatment naive and HBeAg positive before administration of the compound; and wherein if after 76 weeks of administering the compound and the nucleosi(t)e the subject has a pgRNA decline of greater than or equal to 2.5 log₁₀ U/mL from baseline for at least six months prior to the 76^{th} week of administering the compound, administration of the compound and nucleos(t)ide inhibitor continues up to 48 weeks; or

after 76 weeks of administering the compound and the nucleosi(t)e the subject has a pgRNA decline of less than 2.5 log₁₀ U/mL from baseline during the six months prior to the 76^{th} week of administering the compound, administration of the compound is stopped and administration of the nucleos(t)ide inhibitor continues. In some aspects, the method further comprises monitoring the subject for up to twelve weeks after the 76^{th} week of administering the compound for hepatitis B viral DNA concentration and HBeAg concentration if the subject has a pgRNA decline of less than 2.5 log₁₀ U/mL from baseline during the six months prior to the 76^{th} week of administering the compound. In further aspects, the nucleos(t)ide inhibitor is entecavir. In other embodiments, the compound is in a solid dosage form. In some further aspects, the compound is in a solid dispersion. In some aspects, the solid dispersion further comprises a polymer. In further aspects, the solid dispersion further comprises an excipient.

In some embodiments, methods described herein, for example, treating hepatitis B in a subject by administering daily to the subject about 300 mg of a compound represented by:

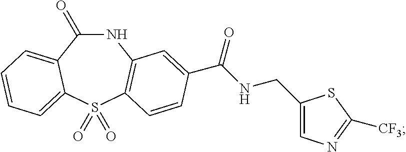 [SMILES: O=C(NCc1cnc(C(F)(F)F)s1)c1ccc2c(c1)NC(=O)c1ccccc1S2(=O)=O]

and administering to the subject a therapeutically effective amount of a nucleos(t)ide inhibitor, such as one selected from the group consisting of entecavir, tenofovir and tenofovir alafenamide fumarate, wherein the 300 mg of the compound is in a pharmaceutical composition disclosed herein comprising a solid dispersion of 11-oxo-N-((2-(trifluoromethyl)thiazol-5-yl)methyl)-10,11-dihydrodibenzo[b,f][1,4]thiazepine-8-carboxamide 5,5-dioxide, in a polymer. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein.

### BRIEF DESCRIPTION OF THE FIGURES

FIGS. 1 and 2 depict thermographs for crystalline 11-oxo-N-((2-(trifluoromethyl)thiazol-5-yl)methyl)-10,11-dihydrodibenzo[b,f][1,4]thiazepine-8-carboxamide 5,5-dioxide.

FIG. 3 depicts the results of thermal analysis of the four dispersion formulations described in Example 1 and Table 1 (i.e., SDDs of Formulations 1-4).

FIG. 4 depicts the diffraction patterns of crystalline 11-oxo-N-((2-(trifluoromethyl)thiazol-5-yl)methyl)-10,11-dihydrodibenzo[b,f][1,4]thiazepine-8-carboxamide 5,5-dioxide.

FIG. 5 depicts PXRD Diffractograms of the four dispersion formulations described in Example 1 and Table 1 (i.e., SDDs of Formulations 1-4).

FIGS. 6 and 7 depict the SEM images of crystalline 11-oxo-N-((2-(trifluoromethyl)thiazol-5-yl)methyl)-10,11-dihydrodibenzo[b,f][1,4]thiazepine-8-carboxamide 5,5-dioxide.

FIGS. 8-11 report the SEM images of the SDD particles, at 5,000× magnification, for the four formulations disclosed in Example 1 and Table 1.

FIG. 12 depicts the results of the SGF/FaSSIF Non-sink dissolution test for compound dispersions, as described in Example 1 and Table 1, compared to bulk crystalline 11-oxo-N-((2-(trifluoromethyl)thiazol-5-yl)methyl)-10,11-dihydrodibenzo[b,f][1,4]thiazepine-8-carboxamide 5,5-dioxide

FIG. 13 reports the SGF/FaSSIF non-sink dissolution test results for Formulation 2 (20:80 Compound 1: Hypromellose Acetate Succinate MG grade SDD) prepared as a suspension, compared to SDD dry powder.

FIG. 14 reports the SGF/FaSSIF non-sink dissolution test results for Formulation 4 (20:80 Compound 1: Hydroxypropyl Methylcellulose Phthalate SDD) prepared as a suspension compared to SDD dry powder.

FIG. 15 reports the PXRD diffractogram of micronized compound compared to bulk compound 11-oxo-N-((2-(trifluoromethyl)thiazol-5-yl)methyl)-10,11-dihydrodibenzo[b,f][1,4]thiazepine-8-carboxamide 5,5-dioxide.

FIG. 16 reports the SGF/FaSSIF non-sink dissolution test for micronized compound compared to bulk crystalline 11-oxo-N-((2-(trifluoromethyl)thiazol-5-yl)methyl)-10,11-dihydrodibenzo[b,f][1,4]thiazepine-8-carboxamide 5,5-dioxide.

FIG. 17 depicts the PXRD diffractograms of Formulation 2 (20:80 Compound 1:HPMCAS-M SDD) after 4 weeks stability.

FIG. 18 depicts the PXRD diffractograms of Formulation 4 (20:80 Compound 1:HPMCP HP-55 SDD) after 4 weeks stability.

FIG. 19 depicts Compression Pressure (M Pa) vs. Solid Fraction for tablets of Formulation 10 (without sodium lauryl sulfate), and

FIG. 20 depicts Compression Pressure (M Pa) vs. Solid Fraction for the tablet comprising Formulation 20 (with sodium lauryl sulfate).

FIG. 21 depicts the Compression Pressure (M Pa) vs. Tensile Strength (M Pa) for Formulation 10 (without sodium lauryl sulfate), and

FIG. 22 depicts Compression Pressure (M Pa) vs. Tensile Strength (M Pa) for Formulation 20 (with sodium lauryl sulfate).

FIG. 23 depicts the release profiles for tablets of Formulation 10 and Formulation 20.

FIG. 24 depicts the plasma concentration of Compound 1 after PO1 dosing at 100 mg/monkey for Formulation 10.

FIG. 25 depicts the plasma concentration of Compound 1 after PO2 dosing at 100 mg/monkey for Formulation 20.

FIG. 26 depicts the PXRD results for the four high drug loading SDDs listed in Table 17.

FIG. 27 depicts the MDSC results for the four high drug loading SDDs listed in Table 17.

FIG. 28 depicts a flow chart of study 201 and 202.

FIG. 29 shows the HBV DNA reduction with Compound 1 in combination with ETV.

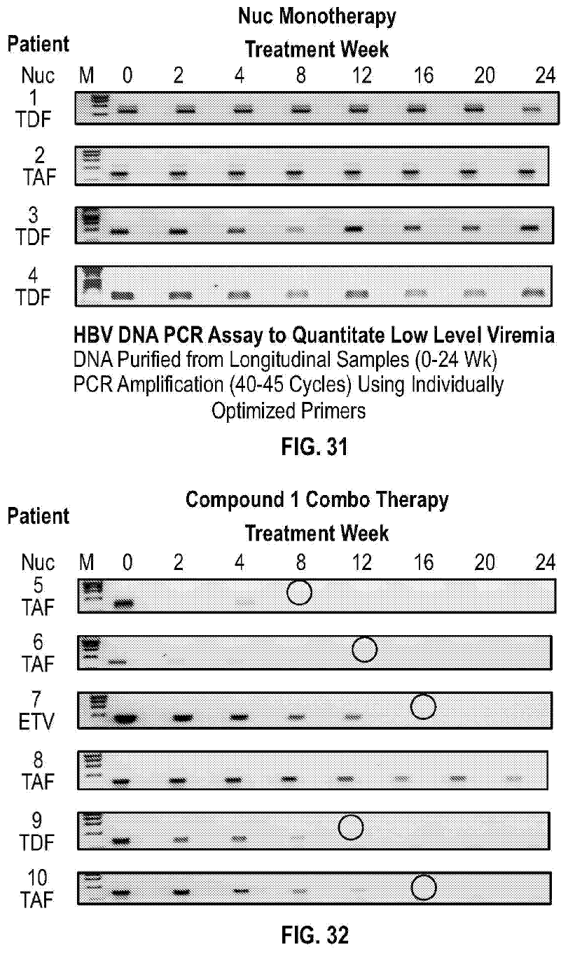

FIG. 30 shows the HBV RNA reduction with Compound 1 in combination with ETV.

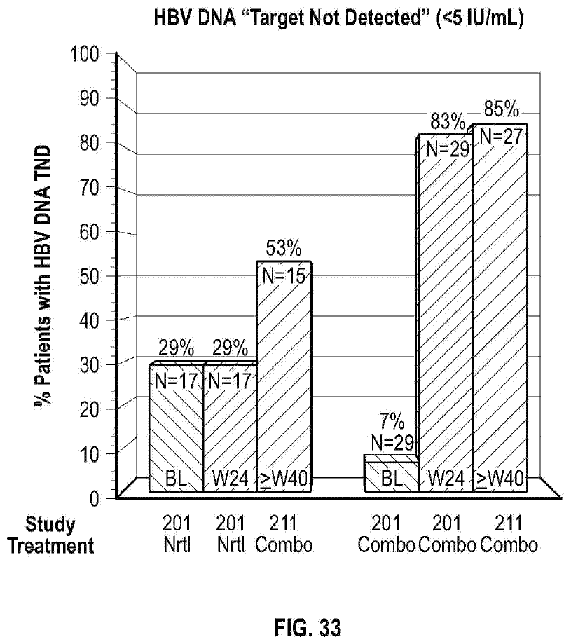

FIG. 31 depicts HBV DNA PCR assay results for Nuc monotherapy (ETV alone).

FIG. 32 depicts HBV DNA PCR assay results for combination therapy of Compound 1 and Nuc therapy.

FIG. 33 shows the percentage of patients with HBV DNA in the open label with HBV DNA at undetectable limits.

FIG. 34a shows the percentage of patients with HBV RNA in the open label with HBV RNA levels less than 35 U/mL and FIG. 34b depicts the percentage of patients with HBV pgRNA levels less than 35 U/mL.

FIG. 35 shows the HBV DNA Log Reduction by treatment week.

FIG. 36 shows the mean HBV RNA Log Reduction by treatment week.

FIG. 37 summarizes the HBeAg reduction levels in patients.

FIG. 38 depicts the correlations between HBV pgRNA reductions and viral antigen declines (patients treated 16-60 weeks with Compound 1 and ETV in Study 202/211).

FIG. 39 summarizes the progression of viral markers in HBV Nrtl-Suppressed patients (patients treated 16-60 weeks with Compound 1 and Nrtl in Study 201/211).

FIG. 40 depicts the log₁₀ change from baseline for patients in Study 202/211.

FIG. 41 depicts the percentage of patients with HBV DNA TND for study 201/211.

FIG. 42 depicts the percentage of patients with composite DNA and pgRNA less than 20 IU/mL.

FIG. 43 depicts the percentage of patients with HBV DNA TND.

FIG. 44 depicts the percentage of patients with DNA and pgRNA less than 20 IU/mL.

### DETAILED DESCRIPTION

The present disclosure provides a pharmaceutical composition that includes a solid dispersion and methods of preparation and use of the same. The solid dispersion includes Compound 1, or a pharmaceutically acceptable salt thereof, and a polymer. The solid dispersion can be prepared by spray drying, which forms a solid spray-dried dispersion. Pharmaceutical compositions of the disclosure can also include pharmaceutically acceptable excipients.

As generally described herein, the present disclosure provides methods of treating hepatitis B in a subject in need thereof, by for example, administering daily to the subject for example, about 300 mg or a dosage amount as disclosed herein of a compound represented by:

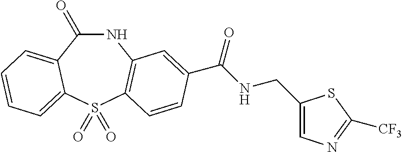 [SMILES: O=C(NCc1cnc(C(F)(F)F)s1)c1ccc2c(c1)NC(=O)c1ccccc1S2(=O)=O]

(also referred to as Compound 1) or a pharmaceutically acceptable salt thereof; and administering to the subject a therapeutically effective amount of a nucleos(t)ide inhibitor selected from the group consisting of entecavir, tenofovir and tenofovir alafenamide fumarate.

### Definitions

As used herein, “Compound 1” refers to 11-oxo-N-((2-(trifluoromethyl)thiazol-5-yl)methyl)-10,11-dihydrodibenzo[b,f][1,4]thiazepine-8-carboxamide 5,5-dioxide, having the structure

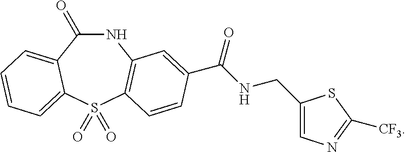 [SMILES: O=C(NCc1cnc(C(F)(F)F)s1)c1ccc2c(c1)NC(=O)c1ccccc1S2(=O)=O]

As used herein, “API” refers to an active pharmaceutical ingredient, e.g., Compound 1, or a pharmaceutically acceptable salt thereof.

As used herein, the term “amorphous” refers to a solid material having no long-range order in the position of its molecules. Amorphous solids are substances in which the molecules are arranged in a random manner so that there is no well-defined arrangement, e.g., molecular packing, and no long-range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid.

As used herein, the expression “substantially amorphous” refers to a solid material having little or no long-range order in the position of its molecules. For example, substantially amorphous materials have less than about 15% crystallinity (e.g., less than about 10% crystallinity or less than about 5% crystallinity). It is also noted that the term “substantially amorphous” includes the descriptor, “amorphous”, which refers to materials having no (0%) crystallinity.

As used herein, the term “dispersion” refers to a disperse system in which one substance, the dispersed phase, is distributed, in discrete units, throughout a second substance (the continuous phase or vehicle or carrier). The size of the dispersed phase can vary considerably (e.g. single molecules or colloidal particles of nanometer dimension up to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in crystalline polymer; a crystalline drug in an amorphous polymer; or a crystalline drug in crystalline polymer. Herein, a solid dispersion can include an amorphous drug in an amorphous polymer, an amorphous drug in crystalline polymer, or a crystalline drug in an amorphous polymer. In some embodiments, a solid dispersion includes the polymer constituting the dispersed phase, and the drug or compound constitutes the continuous phase. Or, a solid dispersion includes the drug constituting the dispersed phase, and the polymer constitutes the continuous phase or carrier.

As used herein, “patient” refers to a mammal, such as a human.

As used herein, the term “therapeutically effective amount” or “effective amount” as used herein refers to the amount of the subject compound that will elicit the biological or medical response of a tissue, system or animal, (e.g. mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

As used herein: \the terms “treating” includes any effect, e.g., lessening, reducing, modulating, or eliminating, via disruption of HBV core protein assembly, that results in the improvement of the disease. “Disruption” includes inhibition of HBV viral assembly and infection.

As used herein, where the term “about” is before a quantitative value, the present teachings also include the specific quantitative value itself, unless specifically stated otherwise. Further, the term “about” refers to a ±10% variation from the nominal value unless otherwise indicated or inferred.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The term “Cₘₐₓ” refers to the maximum concentration of a therapeutic agent (e.g. Compound 1) in the blood (e.g. plasma) following administration of the pharmaceutical composition.

The term “tₘₐₓ” refers to the time in hours when Cₘₐₓ is achieved following administration of the pharmaceutical composition comprising the therapeutic agent (e.g. Compound 1).

“Viraemic infection” refers to Hepatitis B infection associated with presence of virus in the blood (as measured by HBV DNA), and often referred to as active, ongoing or current infection.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

As used herein, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

As used herein, the term “poly(methacrylic acid-co-methyl methacrylate)” (also known as p(MAA-co-MMA)) refers to a class of anionic copolymers, as depicted below, which are a polymerization product of methacrylic acid and methyl methacrylate. The dissolution pH of p(MAA-co-MMA) is dictated by the ratio of monomers utilized in the polymerization. For example, a 1:1 molar ratio of methyl methacrylate and methacrylic acid results in a dissolution above pH 6.0. The resulting polymer is designated as Type A. Type B, synthesized using a 2:1 molar ratio of methyl ester and carboxylic acid monomers, results in a dissolution pH of >7.0.

Chemical structure of p(MAA-co-MMA): n:m=1:1 (type A); n:m=2:1 (type B)

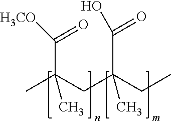

Poly(methacrylic acid-co-methyl methacrylate) polymers are marketed, for example, by Evonik Industries under the EUDRAGIT® tradename.

As used herein, the term “EUDRAGIT® L100” refers to an anionic 1:1 methacrylic acid-methyl methacrylate copolymer (CAS number 25086-15-1), which dissolves in water above pH 6, has a weight average molecular mass of approximately 125,000 g/mol.

As used herein, the term “HPMCAS” refers to hydroxypropylmethylcellulose acetate succinate (CAS 71138-97-1). HPMCAS is typically made from HPMC by esterification with acetic acid anhydride and succinic acid anhydride in acetic acid using a basic catalyst such as sodium acetate. The resulting product, as depicted below, is precipitated by addition of water and subsequently purified by washing with additional water. This reaction sequence leads to a plurality of hydrophobic sites and hydrogen bond acceptor and donor capability.

### Chemical Structure of HPMCAS

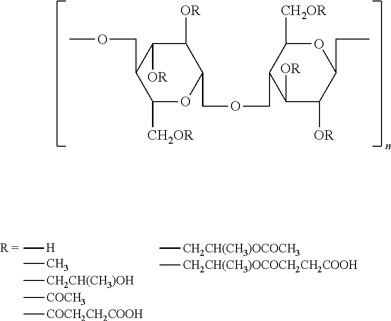

HPMCAS was first introduced by Shin-Etsu Chemical Co., Ltd., Japan, as an enteric coating agent with three substitution levels designated according to the content of acetyl substituents as L, M, or H (e.g., Shin-Etsu AQOAT® LF, MF, HF, LG, MG and HG) The dissolution pH of HPMCAS ranges from about 5.5 (L) to about 6.5 (H) depending on the buffer type used for dissolution. Dow Chemical also markets HPMCAS (e.g., Dow AFFINISOL® 716, 912 and 126) as well as Ashland Chemical (e.g., AQUASOLVE® L, M and H grades). In contrast to HPMC, where substitution levels are specified by the monographs, the range for HPMCAS is not limited to the three commercially available subranges. Manufacturer's specs for these products are shown below in Tables A-C.

TABLE A
Manufacturer's Specs for AQOAT ® HPMCAS by Shin-Estu

| | AS-LG | AS-MG | AS-HG |
|---|---|---|---|
| | AS-LF | AS-MF | AS-HF |
| Viscosity (mm²/s) | 2.4-3.6 | 2.4-3.6 | 2.4-3.6 |
| Heavy metals | ≤10 ppm | ≤10 ppm | ≤10 ppm |
| Arsenic | ≤2 ppm | ≤2 ppm | ≤2 ppm |
| Free succinic acid | ≤1.0% | ≤1.0% | ≤1.0% |
| Loss on drying | ≤5.0% | ≤5.0% | ≤5.0% |
| Residue on ignition | ≤0.20% | ≤0.20% | ≤0.20% |
| Methoxy content | 20.0-24.0% | 21.0-25.0% | 22.0-26.0% |
| Hydroxypropoxy | 5.0-9.0% | 5.0-9.0% | 6.0-10.0% |
| content | | | |
| Acetyl content | 5.0-9.0% | 7.0-11.0% | 10.0-14.0% |
| Succinoyl content | 14.0-18.0% | 10.0-14.0% | 4.0-8.0% |

TABLE B
Manufacturer's Specs for AFFINISOL ® HPMCAS products by Dow

| | 716 | 912 | 128 |
|---|---|---|---|
| Hydroxypropyl | 5.0-9.0% | 5.0-9.0% | 6.0-10.0% |
| Methoxyl | 20-24% | 21-25% | 22-26% |
| viscosity* (cP) | 2.4-3.6 | 2.4-3.6 | 2.4-3.6 |
| Residue on ignition | <0.20% | <0.20% | <0.20% |
| Loss on drying | <5.0% | <5.0% | <5.0% |
| Free acids | <1.0% | <1.0% | <1.0% |
| Acetate substitution | 5.0-9.0% | 7.0-11.0% | 10.0-14.0% |
| Succinate substitution | 14.0-18.0% | 10.0-14.0% | 4.0-8.0% |
| Acetic acid | 0.5% | 0.5% | 0.5% |

*Viscosity determined as a 2% solution in NaOH solution

TABLE C
Manufacturer's Specs for AQUASOLVE ® HPMCAS products by
Ashland

| | LF & LG | MF & MG | HF & HG |
|---|---|---|---|
| Viscosity* (mPa * s) | 2-4-3.6 | 2-4-3.6 | 2-4-3.6 |
| Loss on drying | ≤5% | ≤5% | ≤5% |
| Residue on ignition | ≤0.20% | ≤0.20% | ≤0.20% |
| Heavy metals | ≤10 ppm | ≤10 ppm | ≤10 ppm |
| Arsenic | ≤2 ppm | ≤2 ppm | ≤2 ppm |
| Limit of free succinic | ≤1.0% | ≤1.0% | ≤1.0% |
| and acetic acids | | | |
| Acetyl content | 5-9% | 7-11% | 10-14% |
| Succinoyl content | 14-18% | 10-14% | 4-8% |
| Methoxyl content | 20-24% | 21-25% | 22-26% |
| Hydroxypropoxy | 5-9% | 5-9% | 6-10% |
| content | | | |

*measured for a 2% solution at 20° C.

As used herein the term “HPMCP” refers to hydroxypropyl methylcellulose phthalate (CAS 9050-31-1). The chemical structure of HPMCP, as depicted below, is a phthalic half ester of hydroxypropyl methylcellulose. The threshold pH value for rapid disintegration of HPMCP can be controlled by varying the phthalyl content. HPMCP is marketed, for example, by Shin-Etsu (e.g., HP-55 and HP-50 and HP-55S). Manufacturer's specs for these products are shown below in Table D.

### Chemical Structure of HPMCP

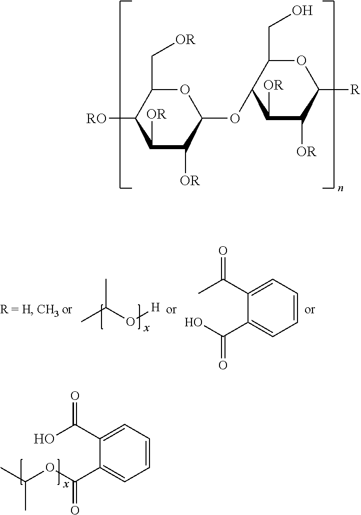

TABLE D
Manufacturer's Specs for HPMCP products by Shin-Etsu

| | HP-55 | HP-55S | HP-50 |
|---|---|---|---|
| Labelled viscosity (cst) | 40 | 170 | 55 |
| Viscosity (cst) | 32-48 | 136-204 | 44-66 |
| Water | ≤5.0% | ≤5.0% | ≤5.0% |
| Residue on ignition | ≤0.20% | ≤0.20% | ≤0.20% |
| Chloride | ≤0.07% | ≤0.07% | ≤0.07% |
| Heavy metals | ≤0.001% | ≤0.001% | ≤0.001% |
| Free phthalic acid | ≤1.0% | ≤1.0% | ≤1.0% |
| Phthalyl content | 27.0-35.0% | 27.0-35.0% | 21.0-27.0% |
| Methoxy content | 18.0-22.0% | 18.0-22.0% | 20.0-24.0% |
| Hydroxypropoxyl | 5.0-9.0% | 5.0-9.0% | 6.0-10.0% |
| content | | | |

### Solid Dispersions:

The pharmaceutical compositions disclosed herein include a solid dispersion off 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, in a polymer. The solid dispersions can be formed by any known technique, e.g., spray drying. In certain embodiments, Compound 1 and a polymer are dissolved in a solvent to firm a mixture, and the solvent is evaporated to form a solid dispersion. In certain embodiments, the solid dispersion is formed without the use of solvents.

Polymers used herein include inert, pharmaceutically acceptable polymers. Suitable polymers include natural or synthetic homopolymers (e.g., polysaccharides) and copolymers (e.g., block copolymers). The polymers typically include a hydrophobic group or region, an/or a group capable of hydrogen bonding. Without being bound by any particular theory, it is believed that hydrogen bonding or hydrophobic interactions between the polymer and Compound 1 impart stabilization, particularly when Compound 1 is in an amorphous or substantially amorphous state.

In certain embodiments, the polymer is a methacrylate or acrylate polymer, for example, a poly(methacrylic acid-co-methyl methacrylate), methacrylic acid/methacrylate copolymer, poly(ethyl methacrylate, poly(propyl methacrylate), or poly(butyl methacrylate). In certain embodiments, the polymer is an acrylate/maleate co-polymer or other functionalized polymer, such as a styrene acrylate.

In certain embodiments, the polymer is a methacrylic acid copolymer selected from the group consisting of: a methacrylic acid copolymer, methacrylic acid—methacrylate copolymer, methacrylic acid—ethyl acrylate copolymer, ammonium methacrylate copolymer, and aminoalkyl methacrylate copolymer. In certain embodiments, the methacrylic acid copolymer is EUDRAGIT® L 100 or EUDRAGIT® L 12,5 (also referred to as, or conforms with: “Methacrylic Acid Copolymer, Type A;” “Methacrylic Acid—Methyl Methacrylate Copolymer (1:1);” “Methacrylic Acid Copolymer L;” “DMF 1242” or “PR-MF 6918”); EUDRAGIT® S 100 and EUDRAGIT® S 12,5 (also referred to as, or conforms with: “Methacrylic Acid Copolymer, Type B;” “Methacrylic Acid—Methyl Methacrylate Copolymer (1:2);” “Methacrylic Acid Copolymer S;” “DMF 1242” or “PR-MF 6918”); EUDRAGIT® L 100-55 (also referred to as, or conforms with: “Methacrylic Acid Copolymer, Type C;” “Methacrylic Acid-Ethyl Acrylate Copolymer (1:1) Type A;” “Dried Methacrylic Acid Copolymer LD;” or “DMF 2584”); EUDRAGIT® L 30 D-55 (also referred to as, or conforms with: “Methacrylic Acid Copolymer Dispersion;” “Methacrylic Acid—Ethyl Acrylate Copolymer (1:1) Dispersion 30 Percent;” “Methacrylic Acid Copolymer LD;” JPE DMF 2584; PR-MF 8216); EUDRAGIT® FS 30 D (also referred to as DMF 13941 or DMF 2006-176); EUDRAGIT® RL 100 (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242” or “PR-MF 6918”); EUDRAGIT® RL PO (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242”); EUDRAGIT® RL 12,5 (also referred to as, or conforms with “Ammonio Methacrylate Copolymer, Type A;” “Ammonio Methacrylate Copolymer (Type A);” “DMF 1242” or “PR-MF 6918”); EUDRAGIT® L 100-55 (also referred to as, or conforms with: “Methacrylic Acid Copolymer, Type C;” “Methacrylic Acid—Ethyl Acrylate Copolymer (1:1) Type A;” “Dried Methacrylic Acid Copolymer LD;” “DMF 2584”); EUDRAGIT® L 30 D-55 (also referred to as, or conforms with: “Methacrylic Acid Copolymer Dispersion” NF “Methacrylic Acid—Ethyl Acrylate Copolymer (1:1) Dispersion 30 Percent;” “Methacrylic Acid Copolymer LD;” “DMF 2584” or “PR-MF 8216”); EUDRAGIT® FS 30 D (also referred to as, or conforms with: “DMF 13941” or “DMF 2006-176”); EUDRAGIT® RL 100 (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242;” or “PR-MF 6918”); EUDRAGIT® RL PO (also referred to as, or conforms with: “Ammonio Methacrylate Copolymer, Type A;” “Ammonio Methacrylate Copolymer (Type A);” “Aminoalkyl Methacrylate Copolymer RS;” or “DMF 1242”); EUDRAGIT® RL 12,5 (also referred to as, or conforms with: polymer conforms to “Ammonio Methacrylate Copolymer, Type A;” “Ammonio Methacrylate Copolymer (Type A);” “DMF 1242” or “PR-MF 6918”); EUDRAGIT® RL 30 D (also referred to as, or conforms with: “Ammonio Methacrylate Copolymer Dispersion, Type A;” “Ammonio Methacrylate Copolymer (Type A);” or “DMF 1242”); EUDRAGIT® RS 100 (also referred to as, or conforms with: “Ammonio Methacrylate Copolymer, Type B;” NF “Ammonio Methacrylate Copolymer (Type B);” “Aminoalkyl Methacrylate Copolymer RS;” “DMF 1242” or “PR-MF 6918”); EUDRAGIT® RS PO (also referred to as, or conforms with: “Ammonio Methacrylate Copolymer, Type B;” “Ammonio Methacrylate Copolymer (Type B);” “Aminoalkyl Methacrylate Copolymer RS;” or “DMF 1242”); EUDRAGIT® RS 12,5 (also referred to as, or conforms with: “Ammonio Methacrylate Copolymer, Type B;” NF polymer conforms to “Ammonio Methacrylate Copolymer (Type B);” “DMF 1242” or “PR-MF 6918”); EUDRAGIT® RS 30 D (also referred to as, or conforms with: “Ammonio Methacrylate Copolymer Dispersion, Type B;” NF polymer conforms to “Ammonio Methacrylate Copolymer (Type B);” or “DMF 1242”); EUDRAGIT® E 100 (also referred to as, or conforms with: “Amino Methacrylate Copolymer;” NF “Basic Butylated Methacrylate Copolymer;” “Aminoalkyl Methacrylate Copolymer E;” “DMF 1242” or “PR-MF 6918”); EUDRAGIT® E PO (also referred to as, or conforms with: “Basic Butylated Methacrylate Copolymer;” “Aminoalkyl Methacrylate Copolymer E;” “Amino Methacrylate Copolymer;” “DMF 1242”); EUDRAGIT® E 12,5 (also referred to as, or conforms with: “Amino Methacrylate Copolymer;” “Basic Butylated Methacrylate Copolymer;” “DMF 1242” or “PR-MF 6918”); EUDRAGIT® NE 30 D (also referred to as, or conforms with: “Ethyl Acrylate and Methyl Methacrylate Copolymer Dispersion;” “Polyacrylate Dispersion 30 Percent;” (“Poly(ethylacrylat-methylmethacrylat)-Dispersion 30%”); “Ethyl Acrylate Methyl Methacrylate Copolymer Dispersion;” “DMF 2822” or “PR-MF 6918”); EUDRAGIT® NE 40 D (also referred to as, or conforms with: DMF 2822); EUDRAGIT® NM 30 D (also referred to as “Polyacrylate Dispersion 30 Percent;” “(Poly(ethylacrylat-methylmethacrylat)-Dispersion 30%);” or “DMF 2822”; PLASTOID® B (also referred to as, or conforms with: “DMF 12102”), or the like.

In certain embodiments, the polymer is a poly(methacrylic acid-co-methyl methacrylate).

In certain embodiments, the polymer is EUDRAGIT® L100, or an equivalent thereof.

In certain embodiments, the polymer is an anionic 1:1 methacrylic acid-methyl methacrylate copolymer with CAS number 25086-15-1.

In certain embodiments, the polymer is an anionic 1:1 methacrylic acid-methyl methacrylate copolymer which dissolves in water above pH 6, having a weight average molecular mass of approximately 125,000 g/mol.

In certain embodiments, the polymer is an anionic 1:1 methacrylic acid-methyl methacrylate copolymer, CAS number 25086-15-1, which dissolves in water above pH 6, having a weight average molecular mass of approximately 125,000 g/mol.

In certain embodiments, the polymer is a cellulosic polymer, or a polymer that has been modified by reaction of at least a portion of the hydroxyl groups on the saccharide repeat units with a compound to form an ester or an ether substituent. In certain embodiments, the cellulosic polymers include: methyl cellulose, sodium carboxymethyl cellulose, hemicellulose, hydroxypropyl methyl cellulose acetate (HPMCA), cellulose acetyate phthalate (CAP), hydroxypropyl methyl cellulose (HPMC), hydroxypropyl methyl cellulose phthalate (HPMCP), hydroxypropyl cellulose (HPC), hydroxypropylmethylcellulose acetate succinate (also known as hypromellose acetate succinate) (HPMCAS), hydroxyethyl methyl cellulose, hydroxyethyl cellulose, hydroxyethyl cellulose acetate, carboxymethylethyl cellulose (CMEC), cellulose acetate succinate (CAS), hydroxypropyl methyl cellulose acetate phthalate (HPMCAP), cellulose acetate trimellitate (CAT), hydroxypropyl methyl cellulose acetate trimellitate (HPMCAT), carboxymethylcellulose acetate butyrate (CMCAB) and hydroxyethyl ethyl cellulose. In certain embodiments, a cellulose derivative polymer is used, such as hydroxypropyl methylcellulose phthalate. In certain embodiments, the cellulosic polymers include: alkyl cellulose, alkyl alkoxyalkyl cellulose, carboxylalkyl cellulose, alkyl carboxyalkyl, preferably selected from carboxymethyl cellulose (CMC) including blocky CMC, methyl hydroxyethyl cellulose, methyl carboxymethyl cellulose, ethyl cellulose, hydroxypropyl cellulose, hydroxypropylmethyl cellulose, hydroxymethyl cellulose, hydroxyethyl cellulose and sodium carboxymethyl cellulose.

In certain embodiments, the polymer is a cellulosic polymer that is at least partially ionized at physiologically relevant pHs. In certain embodiments, the polymer is selected from hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, ethyl carboxymethyl cellulose, cellulose acetate phthalate, carboxymethyl ethyl cellulose, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

In certain embodiments, the polymer is a hydroxypropyl methyl cellulose acetate succinate (HPMCAS).

In certain embodiments, the polymer is a hydroxypropyl methyl cellulose phthalate (HPMCP).

In certain embodiments, the polymer is a starch, lignin, sodium alginate, polyethylene glycol (PEG), polyvinyl pyrollidone (PVP), polyvinyl/alcohol (PVA), β-cyclodextrin, mannitol, chitosan, carrageenan, polyethylene oxide (PEO)/polypropylene glycol (PPG) copolymer, PEG-modified starche, vinyl acetate/vinylpyrrolidone random copolymer, polyvinylpyrrolidone/vinyl acetate copolymer (PVP/VA), polyvinylpyrrolidone (PVP) or polyacrylic acid. In certain embodiments, the polymer is a cationic polymer, such as a deposition-aid polymer, or a cationically-modified cellulose such as cationic hydroxy ethylene cellulose, cationic guar gum, cationic starch, or cationic acrylamide.

All mixtures of the above described polymers, in any ratio, can be used in the formation of the solid dispersion.

A solid dispersion of the disclosure can be formed with one or more of recited polymers described herein. In certain embodiments, the solid dispersions of the disclosure can be formed with two or more of the polymers recited herein.

Various forms or grades of the polymers can be used, for example, based on molecular weight and the pH where the polymer is soluble. For example, HP-55 grade has a higher molecular weight than HP-50 and has a particular pH solubility. In certain embodiments, a MG or HG grade polymer is used in the pharmaceutical composition described herein.

A solid dispersion of the disclosure can be formed by a process, such as spray drying, where Compound 1, or a pharmaceutically acceptable salt thereof, and a polymer are dissolved in a solvent. In certain embodiments, the solvent is an organic solvent. In certain embodiments, the solvent is a mixture of water and an organic solvent. It should be appreciation that Compound 1, or a pharmaceutically acceptable salt thereof, and a polymer (whether one polymer or a mixture of polymers) can be combined in any ratio in the solvent. For example, the ratio of Compound 1, or a pharmaceutically acceptable salt thereof, to the polymer can be about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20 about 85:15, about 90:10, about 95:5, or about 100:0 (by wt %).

Solvents can include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, 1,1,1-trichloroethane, and tetrahydrofuran. Supercritical carbon dioxide can also be used as a solvent, or supercritical carbon dioxide can be used with an organic co-solvent, such as acetone, methanol, ethanol, and/or acetonitrile. Preferred solvents are methanol, acetone, tetrahydrofuran, ethyl acetate, mixtures of these with water, and mixtures thereof.

In certain embodiments, a surfactant and/or a excipient, is added to the mixture. For example, a surfactant such as sodium lauryl sulfate (SLS) can be included in the mixture that is spray dried.

After at least a portion of Compound 1 and the polymer have been dissolved, the solvent can be removed by evaporation or by mixing with a non-solvent. Exemplary processes are spray-drying, spray-coating (e.g., pan-coating and fluidized bed coating), and precipitation by rapid mixing of the compound and polymer mixture with carbon dioxide (CO₂), hexane, heptane, water of appropriate pH, or some other non-solvent. It should be appreciated that solid dispersions of the disclosure can be prepared by methods such as spray drying, melt extrusion, co-precipitation, solvent controlled co-precipitation, freeze drying, and/or spin-coating.

Preferably, removal of the solvent results in a solid dispersion that is substantially homogeneous. To achieve this end, it is generally desirable to rapidly remove the solvent from the solution such as in a process where the solution is atomized, and the compound and the dispersion polymer rapidly solidify.

In certain embodiments, solvent can be removed by spray-drying, e.g., a process that involves breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This can be accomplished by (1) maintaining the pressure in the spray-drying apparatus at a partial; or (2) mixing the liquid droplets with a warm drying gas; or (3) both (1) and (2). In addition, at least a portion of the heat required for evaporation of solvent can be provided by heating the spray solution.

The solvent-bearing feed can be spray-dried under a wide variety of conditions and yet still yield solid dispersions with acceptable properties. For example, various types of nozzles can be used to atomize the spray solution, thereby introducing the spray solution into the spray-dry chamber as a collection of small droplets. Essentially any type of nozzle can be used to spray the solution as long as the droplets that are formed are sufficiently small that they dry sufficiently (due to evaporation of solvent) such that they do not stick to or coat the spray-drying chamber wall.

Methods of spray drying can incorporate the use of an atomizer to break down bulk liquid feed concentrate into fine droplets to facilitate solvent evaporation and particle separations. Atomization techniques can be used to produce particles sizes ranging from 10 to 100 μm. Atomizers can also produce particles ranging from 1 to 5 μm. Atomizers can include four fluid spray nozzles, two-fluid nozzles (pneumatic atomization), pressure nozzles (hydraulic atomization), rotary atomizers (rotating wheel atomization) and ultrasonic atomizer. Any nozzle can be used in the spray-drying technique of the disclosure. Examples of types of nozzles that can be used to form the solid dispersions include the two-fluid nozzle, the fountain-type nozzle, the flat fan-type nozzle, the pressure nozzle and the rotary atomizer.

In some methods, an electro hydro-dynamic or electro-spraying (EHD) atomization can be generally used in spray drying process. In the EHD based atomization method, feed solution is first pumped through a nozzle and the nozzle is applied with a high potential difference. The electrical field formed causes the jet emitted from the nozzle to disintegrate into mono dispersed droplets in the micrometer range. Outlet air temperature is a parameter which could affect the product morphology like particle size, surface roughness, density, stickiness of particles, residual solvent or moisture levels, product yield, etc.

After performing the spray drying process, a secondary drying step of the powder can be used to remove the excess residual solvent because, presence of solvents can plasticize the solid dispersion by increasing molecular mobility and it can result in the development of crystal growth. In certain embodiments, a third (or even more components) along with the polymeric carrier can be added to the organic phase to stabilize the amorphous form of compound during storage. For example, adjuvants such as surfactants or co-solvents can be added to the mixture that forms the solid dispersion to improve the dissolution and physical stability of the compound by improving the wettability and minimize the crystallization of the compound during storage. Examples of surfactants include, among others, sodium lauryl sulfate, polysorbates, and sorbitan esters.

Glidants/drying agents can also be added during the spray drying process to improve the flow property and yield of the powder and to minimize the sticking tendency of particles in the spray drying chamber. Some other additives can also be added in spray drying process like disintegrants, pH modifiers, salt former, complexing agents, etc. The use of colloidal silica can minimize the electrostatic charge generation between powders with the spray dryer wall, leading to increased yield as well as improved flow property of powder. Moreover, porous silica can also work as adsorbents and can play a significant role in solubility enhancement.

Spray drying technology can be an operation in which a liquid stream (organic phase, solution, suspension or emulsion) is constantly divided into very fine droplets (by a process known as atomization) into a compartment where the droplets come in contact with hot gas and get dried into fine particles. The particles are further separated from the drying gas using a cyclone or a bag-filter. Spray driers can operate in open cycle mode for aqueous based or in closed-loop mode for organic based system. Spray drying can be a moderate drying technique (where gentle temperatures and little exposure times are used as compared to other solid dispersion technology like melt extrusion) that yields a powder with reasonable particle size. Moreover, fast drying processes, where the solution is dried within a few seconds or milliseconds, can be important to prevent phase separation between the compound and polymer components.

The spray drying process involves interactions between various formulation variables (feed concentration, solvent type, type of polymer) and process conditions (drying gas flow rate, feed rate, outlet temperature, atomization rate) which can influence the particle characteristics (yield, particle size, residual solvent content, flow property, surface area and release profile) of the solid dispersion.

The spray solution can be delivered to the spray nozzle(s) at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, and can be an inert gas such as nitrogen, nitrogen-enriched air or argon. The drying gas is typically introduced into the drying chamber at a temperature between about 60° C. and about 300° C. and preferably between about 80° C. and about 240° C. Other drying gas temperatures could also be used in forming solid dispersions of the disclosure.

The large surface-to-volume ratio of the droplets and the large driving force for evaporation of solvent leads to rapid solidification times for the droplets. Solidification times can be less than about 20 seconds, can be preferably less than about 10 seconds, and can be more preferably less than 1 second. This rapid solidification can be critical to the particles maintaining a uniform, homogeneous dispersion instead of separating into compound-rich and polymer-rich phases. In a preferred embodiment, the height and volume of the spray-dryer are adjusted to provide sufficient time for the droplets to dry prior to impinging on an internal surface of the spray-dryer.

Following solidification, the solid powder can stay in the spray-drying chamber for about 5 to 60 seconds, further evaporating solvent from the solid powder. The final solvent content of the solid dispersion as it exits the dryer should be low, because this reduces the mobility of compound in the solid dispersion, thereby improving its stability. Generally, the solvent content of the solid dispersion as it leaves the spray-drying chamber should be less than about 10 wt % or less than about 2 wt %.

When the solid dispersion is formed by other methods such by roto-evaporation, precipitation using a non-solvent, spray-coating, melt-congeal, or extrusion processes, the resulting dispersion can be sieved, ground, or otherwise processed to yield a plurality of small particles.

Following its formation, the solid dispersion can be dried to remove residual solvent using suitable drying processes, such as tray drying, vacuum drying, fluid bed drying, microwave drying, belt drying, rotary drying, and other drying processes known in the art.

Preferred secondary drying methods include vacuum drying or tray drying. To minimize chemical degradation during drying, drying can take place under an inert gas such as nitrogen, or can take place under vacuum.

Spray dryers can be of the usual laboratory or commercial type, where suitable spray dryers are manufactured by Buchi Laboratoriums-Technik AG, by the Anhydro Company of Attleboro, Mass. and Niro Atomizer Inc., of Columbia, Md.

In certain embodiments, the solid dispersion is in the form of small particles. The volumetric mean diameter of the particles can be less than about 500 μm, or less than about 100 μm in diameter, less than about 50 μm in diameter or less than about 25 μm in diameter. In certain embodiments, the solid dispersion is in the form of particles that are about 5 μm to about 40 μm, about 10 μm to about 35 μm, or about 15 to about 30 μm in diameter.

Other methods beside spray drying can be used in the formation of the solid dispersion, such as hot-melt extrusion. In hot-melt extrusion, no solvent, or a limited amount of solvent can be used. Hot melt extrusion is a technique for forming a solid dispersion, where a compound is melted or dissolved within a dispersion carrier and mixed to produce and stabilize the amorphous form of the compound. The melt is extruded through a shape forming orifice, and upon rapid cooling, remains a solid, single-phase, glassy amorphous matrix that is shelf-stable. Post extrusion processing can be used to manage the extruded shape making it amendable to processing into a dosage form. Still other methods of forming a solid dispersion include kneading technique, solvent evaporation method, co-precipitation method, melting method, co-grinding method, gel entrapment technique, lyophilization technique, electrospinning method, dropping method solution, and melt agglomeration process. These techniques are all well-known in the art.

b. Properties of Solid Dispersions:

A solid dispersion can be crystalline or amorphous. A solid dispersion can contain a crystalline compound dispersed within a crystalline or semi-crystalline carrier. In other solid dispersions, a carrier can be amorphous rather than crystalline, or it could be a solid crystalline suspension, a solid glassy suspension, or a solid glassy solution. For example, a solid glassy solution containing the drug or compound and carrier can be homogeneous and molecularly dispersed with each other in a single homogeneous phase, and a differential scanning calorimetry (DSC) shows a single glass transition temperature (T_{g}) peak.

In certain embodiments, the solid dispersions of the disclosure have a single glass transition temperature (T_{g}). Two-phase blends also known as solid glassy suspensions contain a compound in a partially miscible state with the polymer and are more prone to undergo phase separation during storage. A solid crystalline suspension can contain a polymer in an amorphous phase while the compound is in a crystalline phase. A DSC of such a suspension shows one T_{g} peak for the polymer and one melting peak for the compound, which indicates no miscibility between the compound and the polymer. To assist in stabilizing the solid dispersion, pharmaceutically suitable carriers like surfactants and stabilizers can be added into the formulation, usually at high concentrations to reduce the molecular mobility and re-crystallization of compound.

In certain embodiments, the solid dispersions of the disclosure are in an amorphous or substantially amorphous state. For example, the solid dispersion can include substantially amorphous Compound 1 or a pharmaceutically acceptable salt thereof, where Compound 1 (or a pharmaceutically acceptable salt thereof) is less than about 15% (e.g., less than about 10% or less than about 5%) crystalline, and at least one polymer. Likewise, the solid dispersion can include amorphous Compound 1 (or a pharmaceutically acceptable salt thereof) and a polymer.

The concentration of Compound 1 or a pharmaceutically acceptable salt thereof in the solid dispersion depends on several factors such as the amount of pharmaceutical composition needed to provide a desired amount of active ingredient, e.g., Compound 1 (or a pharmaceutically acceptable salt thereof) and the desired dissolution profile of the pharmaceutical composition.

In certain embodiments, the pharmaceutical composition comprises a solid dispersion that contains substantially amorphous Compound 1 or a pharmaceutically acceptable salt thereof and a polymer (e.g. HPMCAS or HPMCP), in which the solid dispersion has a mean particle diameter, measured by light scattering, of greater than about 5 μm (e.g., greater than about 6 μm, greater than about 7 μm, greater than about 8 μm, or greater than about 10 μm). In certain embodiments, the pharmaceutical composition of the disclosure includes a solid dispersion comprising substantially amorphous Compound 1 or a pharmaceutically acceptable salt thereof and a polymer, in which the solid dispersion has a mean particle diameter, measured by light scattering, of from about 10 μm to about 35 μm. In certain embodiments, the pharmaceutical composition comprises a solid dispersion comprising substantially amorphous Compound 1 or a pharmaceutically acceptable salt thereof and a polymer, in which the solid dispersion has a mean particle diameter, measured by light scattering, of from about 15 μm to about 35 μm. In certain embodiments, the pharmaceutical composition comprises a solid dispersion comprising amorphous Compound 1 or a pharmaceutically acceptable salt thereof and a polymer, in which the solid dispersion has a mean particle diameter, measured by light scattering, of from about 0.1 μm to about 20 μm.

In some embodiments of the disclosure, the solid dispersion includes substantially amorphous or amorphous Compound 1 (or a pharmaceutically acceptable salt thereof) and a polymer, where the substantially amorphous or amorphous Compound 1 is present in an amount of about 5 wt % to about 80 wt % of the solid dispersion, in an amount of about 10 wt % to about 50 wt % of the solid dispersion, in an amount of about 5 wt % to about 40 wt % of the solid dispersion, or in an amount of about 15 wt % to about 25 wt % of the solid dispersion.

In some embodiments, the solid dispersion includes a polymer from about 10 wt % to about 99 wt %, (e.g., from about 40 wt % to about 95 wt %, from about 40 wt % to about 90 wt %, from about 70 wt % to about 90 wt %, from about 70 wt % to about 85 wt %, or from about 70 wt % to about 80 wt %). In some embodiments, the solid dispersion of the disclosure includes substantially amorphous or amorphous Compound 1 (or a pharmaceutically acceptable salt thereof) in about 10 wt % to about 50 wt %, and a polymer in about 40 wt % to about 90 wt %. In some embodiments, the solid dispersion can include about 20% of substantially amorphous or amorphous Compound 1 (or a pharmaceutically acceptable salt thereof) and about 80 wt % of the polymer.

In some embodiments, solid dispersions of the disclosure are stable compositions. In some embodiments, solid dispersions of the disclosure are stable for at least two weeks, for at least three weeks, for at least four weeks, for at least five weeks, for at least six weeks, for at least seven weeks, for at least eight weeks, or for at least ten weeks.

### Pharmaceutical Compositions:

The present disclosure provides pharmaceutical compositions comprising a solid dispersion of Compound 1 with a polymer, as discussed herein. In general, the pharmaceutical compositions can be formed by combining a solid dispersion of the disclosure with at least one excipient. The resulting pharmaceutical composition then can be formed into a dose unit.

In some embodiments, a pharmaceutical composition of the disclosure includes a solid dispersion of amorphous or substantially amorphous Compound 1 or a pharmaceutically acceptable salt thereof, and a polymer (e.g., poly(methacrylic acid-co-methyl methacrylate), hypromellose acetate succinate (MG grade), hypromellose acetate succinate (HG grade), or hydroxypropyl methylcellulose phthalate).

In some embodiments, the spray-dried composition can be administered to the patient without further processing. However, the spray-dried composition will generally be formulated into a dosage form in conjunction with pharmaceutically acceptable excipients, selected with regards to the desired dosage form. These further excipients will typically be added to the spray dried composition after spray-drying. However, it should be appreciated that surfactants and/or excipients can be added to the mixture prior to spray drying.

The pharmaceutical compositions of the disclosure can also be formed into a wide variety of dosage forms for administration of Compound 1 or a pharmaceutically acceptable salt thereof. Exemplary dosage forms are powders or granules that can be taken orally either dry or reconstituted by addition of water or other liquids to form a paste, slurry, suspension or solution; tablets; capsules; multi-particulates; and pills. Various additives can be mixed, ground, or granulated with the solid dispersions to form a material suitable for the above dosage forms.

Other dosage forms contemplated include aerosols, elixirs, emulsions, gels, inhalers, injections, creams, liniments, ointments, infusions, implants, syrups, tinctures, suspensions, suppositories, otic solutions, ophthalmic solutions, and transdermal preparations.

The pharmaceutical compositions of the present disclosure can be formulated in various forms such that they are delivered as a suspension of particles in a liquid vehicle. Such suspensions can be formulated as a liquid or paste at the time of manufacture, or they can be formulated as a dry powder with a liquid, typically water, which is added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder (a powder that is a solid dispersion of the disclosure, or a powder that is a solid dispersion of the disclosure and at least one excipient) that is reconstituted by simply adding water (or another suitable solvent) and agitating.

The dosage form can be formulated as a liquid and a dry powder (a powder that is a solid dispersion of the disclosure, or a powder that is a solid dispersion of the disclosure and at least one excipient) that are combined and agitated to form the oral suspension. In certain embodiments, the dosage form can be formulated as two powders that are reconstituted by first adding water to one powder to form a solution to which the second powder is combined with agitation to form the suspension, wherein one or both powders contain a solid dispersion of the disclosure.

The pharmaceutical compositions of the present disclosure can also be filled into a suitable capsule, such as a hard gelatin capsule or a soft gelatin capsule, by well-known techniques in the art (see, for example, Remington's The Science and Practice of Pharmacy, 20th Edition, 2000).

The present disclosure provides solid dose forms and unit dose forms comprising a pharmaceutical composition of the disclosure formulated or compressed into a granule, pellet, particle, mini-tablet, and the like. The solid dose forms and unit dose forms include compressed powder pharmaceutical compositions as described above with the addition of one or more functional excipients, for example, a disintegrant, glidant, lubricant, filler and/or a wetting agent to facilitate compression of the powder pharmaceutical composition into a compressed pharmaceutical composition, and to facilitate disintegration and dissolution of the compressed powder. The compressed pharmaceutical composition (solid dose forms) such as granules, pellets, particles, mini-tablets and the like can be formulated into unit dose forms such as tablets, capsules, pouches, sachets, bottles and blister packs containing a one or a plurality of such solid dose forms. The number of solid dose forms required for each unit dose form will depend on the concentration of Compound 1 or a pharmaceutically acceptable salt thereof in each solid dose form (e.g. each granule, pellet or mini-tablet), the size of the unit dose form, (e.g. the volume of the capsule lumen), and the required final amount of Compound 1 or a pharmaceutically acceptable salt thereof required by the unit dose form.

In some embodiments, pharmaceutical compositions of the disclosure can include a solid dispersion of the disclosure (which includes Compound 1 or a pharmaceutically acceptable salt thereof) and at least one excipient that is then formed into a tablet. Methods of tableting can include mixing, blending, granulation, tableting and often coating to form the tablet. Tablets can be defined as the solid unit dosage form of medicament or medicaments with or without suitable diluents and prepared either by molding or by compression. Tablets can include pills, caplets, and/or orally disintegrating tablets. Tablets of the disclosure can be any shape or size. Tablets of the disclosure can be made by known techniques in the art, such as tableting where the powder or granule mixture (e.g., a solid dispersion of the disclosure that contains Compound 1 or a pharmaceutically acceptable salt thereof and optionally, at least one excipient) is prepared, a die mold is filled with the powder or granule mixture, and then the mixture is compressed into a tablet and ejected.

Excipients useful in the pharmaceutical compositions of the present disclosure can be intragranular or extragranular. An excipient is a substance formulated alongside the active ingredient of a medication, included for the purpose of long-term stabilization, bulking up solid formulations that contain potent active ingredients (thus often referred to as “bulking agents”, “fillers”, or “diluents”), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors.

A pharmaceutically acceptable excipient of the disclosure can be a natural polymer. For example, cellulose can be a pharmaceutically acceptable excipient that is incorporated into a pharmaceutical composition comprising a solid dispersion of Compound 1. In certain embodiments, microcrystalline cellulose (MCC) can be incorporated into the pharmaceutical composition.

In certain embodiments, the pharmaceutical composition comprises one, or more than one of any of the following: mannitol, talc, croscarmellose sodium, magnesium stearate, and sodium lauyl sulfate.

Pharmaceutically acceptable excipients of the disclosure can include a disintegrating agent such as maize starch, sodium calcium alginate, alginic acid, microcrystalline cellulose, and a colloidal aluminum silicate.

Pharmaceutically acceptable excipients of the disclosure can include a lubricant. A lubricant can be either hydrophilic or hydrophobic. In some embodiments, pharmaceutical compositions can include one or more of magnesium stearate, calcium stearate, sodium stearate, stearic acid, hydrogenated vegetable oil, mineral oil, polyethylene glycol, sodium lauryl sulfate, glyceryl palmitosterate, glyceryl behenate, sodium benzoate, wax, glyceryl behapate, liquid paraffin, alginic acid, guar gum, sodium starch glycolate, starch corn, sterotex, and sodium stearyl fumarate. Other lubricants include boric acid, sodium benzoate, sodium oleate, sodium acetate, sodium lauryl sulfate, magnesium lauryl sulfate.

Pharmaceutically acceptable excipients of the disclosure can include an anti-adherent and/or a glidant. An anti-adherent can be used to prevent sticking of the pharmaceutical composition to the metal of punches and die walls during processing. Examples of an anti-adherent include talc, cornstarch, colloidal silica, DL-Leucine, sodium lauryl sulfate, and a stearate. A glidant, such as talc, fumed silicon dioxide, starch, colloidal silica, or hydrated sodium silioaluminate, can be incorporated into a pharmaceutical composition of the disclosure.

Other excipients that can be incorporated into pharmaceutical compositions of the disclosure include dextrose, lactose, anhydrous lactose, sorbitol, sucrose, dibasic calcium phosphate, and calcium sulphate dehydrate.

Pharmaceutical compositions of the disclosure can include a binder, such as a dry binder or a wet binder. Examples of binders include gelatin, gum acacia, gum tragacanth, starch, methylcellulose, PVA, and Sod CMC.

Pharmaceutical compositions of the disclosure can include at least one surfactant. It should also be appreciated that the mixture for forming a solid dispersion can include at least one surfactant. Surfactants can be anionic surfactants, cationic surfactants, non-ionic surfactants, or zwitterionic/amphoteric surfactants. Examples include alkyl sulphates, alkyl ethoxylate sulphates, cetrimide, benzalkonium chloride, cetylpyridinium chloride, polyol esters, polyoxyethylene esters, poloxamers, glycol, glycerol esters, sorbitan esters, polysorbates, sorbitan monolaurate, lauryl diglucoside, and sucrose monostreate.

Pharmaceutical compositions of the disclosure can include a wetting agent. For example, a wetting agent can be a hydrophilic colloid such as alginate, bentonite, cellulose derivatives, or tragacanth. Examples of possible surfactants include SLS, polysorbates, and sorbitan esters.

Pharmaceutical compositions of the disclosure can include coloring agents, flavoring agents, and/or sweetening agents.

Pharmaceutical compositions of the disclosure can include a spray dried dispersion comprising a polymer and Compound 1 or a pharmaceutically acceptable salt thereof, microcrystalline cellulose, mannitol, talc, croscarmellose sodium, and magnesium stearate. In various embodiments, sodium lauryl sulfate can be incorporated into the pharmaceutical composition. The pharmaceutical composition can be compressed into a tablet and then administered to a patient or individual.

Pharmaceutical compositions of the disclosure can be administered by routes of administration. For example, pharmaceutical compositions of the disclosure can be administered systemically, parenterally, or locally. Pharmaceutical compositions of the disclosure can be administered via oral, sublingual/buccal, rectal, parenteral, intravenous, intramuscular, subcutaneous, intraventricular, transdermal, topical, inhalation, and/or intranasal.

In some embodiments of the disclosure, pharmaceutical compositions are formed into dosing forms that can be administered by inhalation. In some embodiments of the disclosure, pharmaceutical compositions are formed into dosing forms that can be administered by an injection.

In some embodiments of the disclosure, pharmaceutical compositions are formed into dosing forms that can be administered orally, for example by the mouth (Per os (P.O.)). Oral administration can be in the form of a tablet, capsule, chewable capsule, time-release or sustained-release tablets and capsules, and/or powders or granules. Oral administration can typically involve swallowing so that the compound enters the gastrointestinal tract (GIT). Additional dosage forms or dosing units for oral administration include solid formulations such as tablets, capsules containing particulates or powders, sachets, vials, powders, granules, lozenges, reconstitutable powders and liquid preparations (such as suspensions, emulsions and elixirs).

Oral dosage forms can contain further excipients such as binding agents (for example syrup, acacia, gelatin, sorbitol, starch, PVP, HPMC, and tragacanth); fillers (for example lactose, sugar, maize-starch, calcium phosphate, sorbitol and glycine); tableting lubricants (for example magnesium stearate); and disintegrants (for example starch, sodium starch glycollate and microcrystalline cellulose). In addition, the oral dosage form can contain preservatives, anti-oxidant, flavors, granulation binders, wetting agents and colorants.

Tablets can be prepared using standard technology familiar to the formulation chemist, for example by direct compression, granulation, melt congealing and extrusion. The tablet can be coated or uncoated. The tablet can be formulated to be immediate or controlled release. Controlled release formulations include delayed, sustained, pulsed or dual-release. Suitable tableting excipients are described in the Handbook of Pharmaceutical Excipients, Pharmaceutical Press, 1986, published by The American Pharmaceutical Association and The Royal Pharmaceutical Society of Great Britain. Typical tableting excipients include: carriers (for example lactose and starch), lubricating agents (for example magnesium stearate), binding agents, wetting agents, colorants, flavorings, glidants and disintegrants (for example croscarmellose sodium).

Alternatively, the pharmaceutical composition can be formulated into a unit dose form containing the solid dispersion or a unit dose form formulated to contain a compressed solid dose form of the solid dispersion in addition to one or more additional functional excipients, for example, a wetting agent and/or lubricant to enable the compression of the solid dispersion into granules, pellets, particles, or one or more mini-tablets, the pharmaceutical composition and/or the unit dose form comprising the specified ingredients in the specified amounts. The pharmaceutical composition is capable of being formulated into a unit dose form, for example, a tablet, capsule, sachet, troches, blister pack and the like containing the powder and/or compressed form of the pharmaceutical composition of the present disclosure.

In certain embodiments, a pharmaceutical composition of the present disclosure can be formed with a solid dispersion of the disclosure (which contains Compound 1 or a pharmaceutically acceptable salt thereof) and optionally, at least one excipient, where the pharmaceutical composition is compressed into a dose form (e.g., a tablet or a capsule). In certain embodiments, the dose form can contain about 1 mg to about 1000 mg of Compound 1 or a pharmaceutically acceptable salt thereof, about 5 mg to about 900 mg of Compound 1 or a pharmaceutically acceptable salt thereof, about 50 mg to about 800 mg of Compound 1 or a pharmaceutically acceptable salt thereof, about 100 mg to about 700 mg of Compound 1 or a pharmaceutically acceptable salt thereof, about 200 mg to about 700 mg of Compound 1 or a pharmaceutically acceptable salt thereof, about 300 mg to about 600 mg of Compound 1 or a pharmaceutically acceptable salt thereof, or about 400 mg to about 500 mg of Compound 1 or a pharmaceutically acceptable salt thereof.

In certain embodiments, the dose form can contain about 1 mg to about 150 mg of Compound 1 or a pharmaceutically acceptable salt thereof, about 5 mg to about 150 mg of Compound 1 or a pharmaceutically acceptable salt thereof, about 10 mg to about 150 mg of Compound 1 or a pharmaceutically acceptable salt thereof, about 30 mg to about 150 mg of Compound 1 or a pharmaceutically acceptable salt thereof, about 50 mg to about 150 mg of Compound 1 or a pharmaceutically acceptable salt thereof, or about 75 mg to about 125 mg of Compound 1 or a pharmaceutically acceptable salt thereof.

In certain embodiments, the dose form can contain about 200 mg to about 400 mg of Compound 1 or a pharmaceutically acceptable salt thereof, about 250 mg to about 350 mg of Compound 1 or a pharmaceutically acceptable salt thereof, or about 300 mg of Compound 1 or a pharmaceutically acceptable salt thereof.

In certain embodiments, a pharmaceutical composition of the present disclosure is formed with a solid dispersion of the disclosure (which contains Compound 1 or a pharmaceutically acceptable salt thereof) and optionally, at least one excipient, where the pharmaceutical composition is compressed into a dose form, and the dose form contains about 0.1 to about 5 mg, about 0.25 to about 5 mg, about 0.5 to about 5 mg, about 0.5 to about 4 mg, about 0.5 to about 3 mg, about 1 to about 5 mg, about 1.5 to about 5 mg, about 2 to about 5 mg, and about 3 to about 5 mg.

Dosing units of the disclosure will depend on the various factors such as effective dose of Compound 1 or a pharmaceutically acceptable salt thereof, and the frequency and route of administration.

### Methods of Treatment:

The present disclosure provides a method of treating Hepatitis B (HBV) in a patient in need thereof, comprising: administering to the patient a therapeutically effective amount of a pharmaceutical composition as described herein.

In some embodiments, provided herein is a compound represented by:

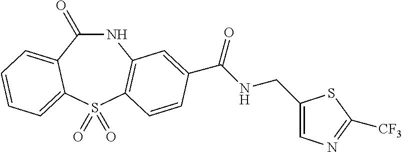 [SMILES: O=C(NCc1cnc(C(F)(F)F)s1)c1ccc2c(c1)NC(=O)c1ccccc1S2(=O)=O]

(also referred to as Compound 1) or a pharmaceutically acceptable salt thereof, for the treatment of hepatitis B in a subject.

In some embodiments, a pharmaceutical composition comprises Compound 1 or pharmaceutically acceptable salt thereof, and a pharmaceutically acceptable excipient. In other embodiments, a composition comprises Compound 1 or pharmaceutically acceptable salt thereof, and a pharmaceutically acceptable excipient.

In some embodiments, provided herein are methods of treating hepatitis B in a subject in need thereof, comprising administering to the subject an effective amount of a Compound 1 or a pharmaceutically acceptable salt thereof.

In some embodiments, provided herein are methods of treating hepatitis B in a subject in need thereof, comprising administering to the subject about 100 mg to about 500 mg, e.g., about 225 mg, about 250 mg, about 275 mg, about 300 mg, about 325 mg, about 350 mg, about 375 mg, about 400 mg, about 425 mg, or about 450 mg or more, e.g. daily, of Compound 1 or a pharmaceutically acceptable salt thereof, and optionally administering a therapeutically effective amount of a nucleos(t)ide inhibitor such as contemplated herein. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein.

For example, provided herein are methods of treating hepatitis B in a subject in need thereof, comprising administering to the subject about 100 mg to about 500 mg, e.g., about 225 mg, about 250 mg, about 275 mg, about 300 mg, about 325 mg, about 350 mg, about 375 mg, about 400 mg, about 425 mg, or about 450 mg or more, e.g. daily 300 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject 300 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 150 mg to about 200 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 200 mg to about 225 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 200 mg to about 250 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 225 mg to about 250 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 250 mg to about 300 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 300 mg to about 350 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 280 mg to about 300 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 280 mg to about 320 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 300 mg to about 325 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 325 mg to about 350 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 350 mg to about 375 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 375 mg to about 400 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

In some embodiments, provided herein are methods of treating hepatitis B in a subject in need thereof, comprising administering to the subject about 400 mg to about 425 mg of Compound 1 or a pharmaceutically acceptable salt thereof and administering to the subject a therapeutically effective amount of a nucleos(t)ide inhibitor selected from the group consisting of entecavir, tenofovir and tenofovir alafenamide fumarate.

Exemplary compounds of the disclosure may be synthesized from the following known starting materials using methods known to one skilled in the art or certain references.

Provided herein are methods of treating hepatitis B (HBV). Provided herein are methods of treating an acute HBV infection (or a new infection). An acute hepatitis B infection may last up to six months (with or without symptoms) and infected persons are able to pass the virus to others during this time. Provided herein are methods of treating chronic HBV, which is defined as a condition where the virus is not eliminated after six months. Subjects who test positive for the persistence of HBsAg for more than six months (after their first blood test result) are diagnosed as having a chronic HBV infection.

Subjects may be diagnosed with HBV by the results from a serological assay, which is an assay that detect the presence of either antigens or antibodies, typically in serum or plasma but also in capillary/venous whole blood and oral fluid. These include rapid diagnostic tests (RDTs), and laboratory-based immunoassays, e.g. enzyme immunoassays (EIAs), chemiluminescence immunoassays (CLIAs), and electrochemiluminescence immunoassays (ECLs). A positive or reactive Hepatitis B surface antigen HBsAg test result means that the subject is infected with hepatitis B. This test can detect the actual presence of the hepatitis B virus (called the “surface antigen”) in blood. If a person tests positive, then further testing would be needed to determine if this is a new acute infection or a chronic hepatitis B infection. A positive HBsAg test result means that the subject is infected and can spread the hepatitis B virus to others through blood.

A positive or reactive anti-HBs (or HBsAb) (Hepatitis B surface antibody) test result indicates that a subject is protected against the hepatitis B virus. This protection can be the result of receiving the hepatitis B vaccine or successfully recovering from a past hepatitis B infection. A positive anti-HBs (or HBsAb) test result means the subject is immune and protected against the hepatitis B virus and cannot be infected.

A positive or reactive anti-HBc (or HBcAb) (Hepatitis B core antibody) test result indicates a past or current hepatitis B infection. The core antibody does not provide any protection against the hepatitis B virus (unlike the surface antibody described above).

A positive test for the hepatitis B e-antigen (HBeAg), a protein from the hepatitis B virus that circulates in blood, indicates that there is an active infection with the hepatitis B virus and the virus is actively multiplying.

HBV DNA and HBV RNA are HBV viral genomes that can be detected and quantified in serum by nucleic acid testing (NAT). Serum HBV DNA and HBV RNA is measured in international units (IU)/mL as the recognized international standard or copies/ml by nucleic acid testing (NAT) technologies.

In some embodiments, provided herein are methods treating hepatitis B in a subject by administering daily to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, before the subject is administered Compound 1, the subject is virologically suppressed for at least 6 months. In some embodiments, before the subject is administered Compound 1, the subject is virologically suppressed for at least 5 months. In some embodiments, before the subject is administered Compound 1, the subject is virologically suppressed for at least 4 months. In some embodiments, before the subject is administered Compound 1, the subject is virologically suppressed for at least 3 months. In some embodiments, before the subject is administered Compound 1, the subject is virologically suppressed for at least 2 months. In some embodiments, before the subject is administered Compound 1, the subject is virologically suppressed for at least 1 month.

In some embodiments, before the subject is administered Compound 1, the subject is virologically suppressed for at least 6 months and the subject had previously been administer a nucleos(t)ide inhibitor alone. In some embodiments, before the subject is administered Compound 1, the subject is virologically suppressed for at least 5 months and the subject had previously been administer a nucleos(t)ide inhibitor alone. In some embodiments, before the subject is administered Compound 1, the subject is virologically suppressed for at least 4 months and the subject had previously been administer a nucleos(t)ide inhibitor alone. In some embodiments, before the subject is administered Compound 1, the subject is virologically suppressed for at least 3 months and the subject had previously been administer a nucleos(t)ide inhibitor alone. In some embodiments, before the subject is administered Compound 1, the subject is virologically suppressed for at least 2 months and the subject had previously been administer a nucleos(t)ide inhibitor alone. In some embodiments, before the subject is administered Compound 1, the subject is virologically suppressed for at least 1 month and the subject had previously been administered a nucleos(t)ide inhibitor alone.

In other embodiments, before the subject is administered Compound 1, the subject has not been previously administered a nucleos(t)ide inhibitor. In some embodiments, before the subject is administered Compound 1, the subject has not been previously administered a nucleos(t)ide inhibitor for at least 1 month. In some embodiments, before the subject is administered Compound 1, the subject has not been previously administered a nucleos(t)ide inhibitor for at least 2 months. In some embodiments, before the subject is administered Compound 1, the subject has not been previously administered a nucleos(t)ide inhibitor for at least 3 months. In some embodiments, before the subject is administered Compound 1, the subject has not been previously administered a nucleos(t)ide inhibitor for at least 4 months. In some embodiments, before the subject is administered Compound 1, the subject has not been previously administered a nucleos(t)ide inhibitor for at least 5 months. In some embodiments, before the subject is administered Compound 1, the subject has not been previously administered a nucleos(t)ide inhibitor for at least 6 months. In some embodiments, before the subject is administered Compound 1, the subject has not been previously administered a nucleos(t)ide inhibitor for at least 1 year. In some embodiments, before the subject is administered Compound 1, the subject has not been previously administered a nucleos(t)ide inhibitor for at least 2 years.

In some embodiments, before the subject is administered Compound 1, it is determined that the subject has detectable levels of hepatitis B viral DNA prior to administration. In some embodiments, before the subject is administered Compound 1, it is determined that the subject is positive for the hepatitis B e-antigen (HBeAg) prior to administration.

In some embodiments, the subject is HBeAg negative prior to daily administration.

In some embodiments, provided herein are methods treating hepatitis B in a subject by administering daily for at least 2 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate.

In some embodiments, provided herein are methods treating hepatitis B in a subject by administering daily for at least 4 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 8 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 12 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 16 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 24 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 28 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 32 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 40 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 44 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate.

In some embodiments, provided herein are methods treating hepatitis B in a subject by administering daily for at least 72 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 76 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein.

In some embodiments, provided herein are methods treating hepatitis B in a subject by administering daily for at least 48 weeks to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 1 year to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 18 months to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 2 years to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 2.5 years to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate. In some embodiments, provided herein are methods treating hepatitis B in a subject by administering daily for at least 3 years to the subject for example, about 300 mg or a dosage amount as disclosed herein of Compound 1 or a pharmaceutically acceptable salt thereof; and administering to the subject a nucleos(t)ide inhibitor such as entecavir, tenofovir or tenofovir alafenamide fumarate.

In some embodiments, the subject is assessed after a set time period for HBeAg, HBsAg, HBV DNA, and HBV RNA levels, amounts, or concentrations after the subject has undergone administered daily of for example, about 300 mg or a dosage amount as disclosed herein of Compound 1 or a pharmaceutically acceptable salt thereof; and daily administration of a therapeutically effective amount of a nucleos(t)ide inhibitor such as entecavir, tenofovir or tenofovir alafenamide fumarate. The set time period can be about 2 weeks, about 4 weeks, about 6 weeks, about 8 weeks, about 12 weeks, about 16 weeks, about 24 weeks, about 28 weeks, about 32 weeks, about 40 weeks, about 44 weeks, about 48 weeks, about 50 weeks, about 12 months, about 18 months, about 24 months, about 30 months, about 36 months, about 42 months, about 48 months, or about 54 months.

In some embodiments, the HBeAg positive subject after 2 weeks of daily administration (of the combination of Compound 1 and a nucleos(t)ide inhibitor described herein), has sustained HBeAg loss of <0.11 PEI units/mL. In some embodiments, the HBeAg positive subject after 4 weeks of daily administration (of the combination of Compound 1 and a nucleos(t)ide inhibitor described herein), has sustained HBeAg loss of <0.11 PEI units/mL. In some embodiments, the HBeAg positive subject after 8 weeks of daily administration (of the combination of Compound 1 and a nucleos(t)ide inhibitor described herein), has sustained HBeAg loss of <0.11 PEI units/mL. In some embodiments, the HBeAg positive subject after 12 weeks of daily administration (of the combination of Compound 1 and a nucleos(t)ide inhibitor described herein), has sustained HBeAg loss of <0.11 PEI units/mL. In some embodiments, the HBeAg positive subject after 24 weeks of daily administration (of the combination of Compound 1 and a nucleos(t)ide inhibitor described herein), has sustained HBeAg loss of <0.11 PEI units/mL. In some embodiments, the HBeAg positive subject after 30 weeks of daily administration (of the combination of Compound 1 and a nucleos(t)ide inhibitor described herein), has sustained HBeAg loss of <0.11 PEI units/mL. In some embodiments, the HBeAg positive subject after 34 weeks of daily administration (of the combination of Compound 1 and a nucleos(t)ide inhibitor described herein), has sustained HBeAg loss of <0.11 PEI units/mL. In some embodiments, the HBeAg positive subject after 40 weeks of daily administration (of the combination of Compound 1 and a nucleos(t)ide inhibitor described herein), has sustained HBeAg loss of <0.11 PEI units/mL. In some embodiments, the HBeAg positive subject after 44 weeks of daily administration (of the combination of Compound 1 and a nucleos(t)ide inhibitor described herein), has sustained HBeAg loss of <0.11 PEI units/mL. In some embodiments, the HBeAg positive subject after 12 months of daily administration (of the combination of Compound 1 and a nucleos(t)ide inhibitor described herein), has sustained HBeAg loss of <0.11 PEI units/mL. In some embodiments, the HBeAg positive subject after 18 months of daily administration (of the combination of Compound 1 and a nucleos(t)ide inhibitor described herein), has sustained HBeAg loss of <0.11 PEI units/mL. In some embodiments, the HBeAg positive subject after 24 months of daily administration (of the combination of Compound 1 and a nucleos(t)ide inhibitor described herein), has sustained HBeAg loss of <0.11 PEI units/mL. In some embodiments, the HBeAg positive subject after 30 months of daily administration (of the combination of Compound 1 and a nucleos(t)ide inhibitor described herein), has sustained HBeAg loss of <0.11 PEI units/mL. In some embodiments, the HBeAg positive subject after 36 months of daily administration (of the combination of Compound 1 and a nucleos(t)ide inhibitor described herein), has sustained HBeAg loss of <0.11 PEI units/mL. In some embodiments, the HBeAg positive subject after 40 months of daily administration (of the combination of Compound 1 and a nucleos(t)ide inhibitor described herein), has sustained HBeAg loss of <0.11 PEI units/mL. In some embodiments, the HBeAg positive subject after 44 months of daily administration (of the combination of Compound 1 and a nucleos(t)ide inhibitor described herein), has sustained HBeAg loss of <0.11 PEI units/mL. In some embodiments, the HBeAg positive subject after 46 months of daily administration (of the combination of Compound 1 and a nucleos(t)ide inhibitor described herein), has sustained HBeAg loss of <0.11 PEI units/mL.

In some embodiments, after 2 weeks of daily administration (of the combination of Compound 1 and a nucleos(t)ide inhibitor described herein) the subject has a reduction of HBeAg and/or HBsAg. In some embodiments, after 4 weeks of daily administration (of the combination of Compound 1 and a nucleos(t)ide inhibitor described herein) the subject has a reduction of HBeAg and/or HBsAg. In some embodiments, after 8 weeks of daily administration (of the combination of Compound 1 and a nucleos(t)ide inhibitor described herein) the subject has a reduction of HBeAg and/or HBsAg. In some embodiments, after 12 weeks of daily administration (of the combination of Compound 1 and a nucleos(t)ide inhibitor described herein) the subject has a reduction of HBeAg and/or HBsAg. In some embodiments, after 16 weeks of daily administration (of the combination of Compound 1 and a nucleos(t)ide inhibitor described herein) the subject has a reduction of HBeAg and/or HBsAg. In some embodiments, after 20 weeks of daily administration (of the combination of Compound 1 and a nucleos(t)ide inhibitor described herein) the subject has a reduction of HBeAg and/or HBsAg. In some embodiments, after 24 weeks of daily administration (of the combination of Compound 1 and a nucleos(t)ide inhibitor described herein) the subject has a reduction of HBeAg and/or HBsAg. In some embodiments, after 28 weeks of daily administration (of the combination of Compound 1 and a nucleos(t)ide inhibitor described herein) the subject has a reduction of HBeAg and/or HBsAg. In some embodiments, after 32 weeks of daily administration (of the combination of Compound 1 and a nucleos(t)ide inhibitor described herein) the subject has a reduction of HBeAg and/or HBsAg. In some embodiments, after 36 weeks of daily administration (of the combination of Compound 1 and a nucleos(t)ide inhibitor described herein) the subject has a reduction of HBeAg and/or HBsAg. In some embodiments, after 40 weeks of daily administration (of the combination of Compound 1 and a nucleos(t)ide inhibitor described herein) the subject has a reduction of HBeAg and/or HBsAg. In some embodiments, after 44 weeks of daily administration (of the combination of Compound 1 and a nucleos(t)ide inhibitor described herein) the subject has a reduction of HBeAg and/or HBsAg. In some embodiments, after 12 months of daily administration (of the combination of Compound 1 and a nucleos(t)ide inhibitor described herein) the subject has a reduction of HBeAg and/or HBsAg. In some embodiments, after 18 months of daily administration (of the combination of Compound 1 and a nucleos(t)ide inhibitor described herein) the subject has a reduction of HBeAg and/or HBsAg. In some embodiments, after 24 months of daily administration (of the combination of Compound 1 and a nucleos(t)ide inhibitor described herein) the subject has a reduction of HBeAg and/or HBsAg. In some embodiments, after 30 months of daily administration (of the combination of Compound 1 and a nucleos(t)ide inhibitor described herein) the subject has a reduction of HBeAg and/or HBsAg. In some embodiments, after 36 months of daily administration (of the combination of Compound 1 and a nucleos(t)ide inhibitor described herein) the subject has a reduction of HBeAg and/or HBsAg. In some embodiments, after 42 months of daily administration (of the combination of Compound 1 and a nucleos(t)ide inhibitor described herein) the subject has a reduction of HBeAg and/or HBsAg. In some embodiments, after 48 months of daily administration (of the combination of Compound 1 and a nucleos(t)ide inhibitor described herein) the subject has a reduction of HBeAg and/or HBsAg.

In some embodiments, after 2 weeks of daily administration (of the combination of Compound 1 and a nucleos(t)ide inhibitor described herein) the subject has a loss or stable reduction of HBsAg to ≤100 IU/mL. In some embodiments, after 4 weeks of daily administration (of the combination of Compound 1 and a nucleos(t)ide inhibitor described herein) the subject has a loss or stable reduction of HBsAg to ≤100 IU/mL. In some embodiments, after 8 weeks of daily administration (of the combination of Compound 1 and a nucleos(t)ide inhibitor described herein) the subject has a loss or stable reduction of HBsAg to ≤100 IU/mL. In some embodiments, after 12 weeks of daily administration (of the combination of Compound 1 and a nucleos(t)ide inhibitor described herein) the subject has a loss or stable reduction of HBsAg to ≤100 IU/mL. In some embodiments, after 16 weeks of daily administration (of the combination of Compound 1 and a nucleos(t)ide inhibitor described herein) the subject has a loss or stable reduction of HBsAg to ≤100 IU/mL. In some embodiments, after 24 weeks of daily administration (of the combination of Compound 1 and a nucleos(t)ide inhibitor described herein) the subject has a loss or stable reduction of HBsAg to ≤100 IU/mL. In some embodiments, after 30 weeks of daily administration (of the combination of Compound 1 and a nucleos(t)ide inhibitor described herein) the subject has a loss or stable reduction of HBsAg to ≤100 IU/mL. In some embodiments, after 36 weeks of daily administration (of the combination of Compound 1 and a nucleos(t)ide inhibitor described herein) the subject has a loss or stable reduction of HBsAg to ≤100 IU/mL. In some embodiments, after 44 weeks of daily administration (of the combination of Compound 1 and a nucleos(t)ide inhibitor described herein) the subject has a loss or stable reduction of HBsAg to ≤100 IU/mL. In some embodiments, after 48 weeks of daily administration (of the combination of Compound 1 and a nucleos(t)ide inhibitor described herein) the subject has a loss or stable reduction of HBsAg to ≤100 IU/mL. In some embodiments, after 12 months of daily administration (of the combination of Compound 1 and a nucleos(t)ide inhibitor described herein) the subject has a loss or stable reduction of HBsAg to ≤100 IU/mL. In some embodiments, after 18 months of daily administration (of the combination of Compound 1 and a nucleos(t)ide inhibitor described herein) the subject has a loss or stable reduction of HBsAg to <100 IU/mL. In some embodiments, after 24 months of daily administration (of the combination of Compound 1 and a nucleos(t)ide inhibitor described herein) the subject has a loss or stable reduction of HBsAg to <100 IU/mL. In some embodiments, after 30 months of daily administration (of the combination of Compound 1 and a nucleos(t)ide inhibitor described herein) the subject has a loss or stable reduction of HBsAg to <100 IU/mL. In some embodiments, after 36 months of daily administration (of the combination of Compound 1 and a nucleos(t)ide inhibitor described herein) the subject has a loss or stable reduction of HBsAg to <100 IU/mL. In some embodiments, after 42 months of daily administration (of the combination of Compound 1 and a nucleos(t)ide inhibitor described herein) the subject has a loss or stable reduction of HBsAg to <100 IU/mL. In some embodiments, after 48 months of daily administration (of the combination of Compound 1 and a nucleos(t)ide inhibitor described herein) the subject has a loss or stable reduction of HBsAg to <100 IU/mL. In some embodiments, after 52 months of daily administration (of the combination of Compound 1 and a nucleos(t)ide inhibitor described herein) the subject has a loss or stable reduction of HBsAg to <100 IU/mL.

In some embodiments, after 2 weeks of daily administration (of the combination of Compound 1 and a nucleos(t)ide inhibitor described herein) the subject has sustained viral suppression (e.g. below the limits of detection=20 IU/mL). In some embodiments, the subject after 4 weeks of daily administration (of the combination of Compound 1 and a nucleos(t)ide inhibitor described herein) the subject has sustained viral suppression. In some embodiments, the subject after 8 weeks of daily administration (of the combination of Compound 1 and a nucleos(t)ide inhibitor described herein) the subject has sustained viral suppression. In some embodiments, the subject after 12 weeks of daily administration (of the combination of Compound 1 and a nucleos(t)ide inhibitor described herein) the subject has sustained viral suppression. In some embodiments, the subject after 16 weeks of daily administration (of the combination of Compound 1 and a nucleos(t)ide inhibitor described herein) the subject has sustained viral suppression. In some embodiments, the subject after 18 weeks of daily administration (of the combination of Compound 1 and a nucleos(t)ide inhibitor described herein) the subject has sustained viral suppression. In some embodiments, the subject after 24 weeks of daily administration (of the combination of Compound 1 and a nucleos(t)ide inhibitor described herein) the subject has sustained viral suppression. In some embodiments, the subject after 30 weeks of daily administration (of the combination of Compound 1 and a nucleos(t)ide inhibitor described herein) the subject has sustained viral suppression. In some embodiments, the subject after 36 weeks of daily administration (of the combination of Compound 1 and a nucleos(t)ide inhibitor described herein) the subject has sustained viral suppression. In some embodiments, the subject after 42 weeks of daily administration (of the combination of Compound 1 and a nucleos(t)ide inhibitor described herein) the subject has sustained viral suppression. In some embodiments, the subject after 44 weeks of daily administration (of the combination of Compound 1 and a nucleos(t)ide inhibitor described herein) the subject has sustained viral suppression. In some embodiments, the subject after 12 months of daily administration (of the combination of Compound 1 and a nucleos(t)ide inhibitor described herein) the subject has sustained viral suppression. In some embodiments, the subject after 18 months of daily administration (of the combination of Compound 1 and a nucleos(t)ide inhibitor described herein) the subject has sustained viral suppression. In some embodiments, the subject after 24 months of daily administration (of the combination of Compound 1 and a nucleos(t)ide inhibitor described herein) the subject has sustained viral suppression. In some embodiments, the subject after 30 months of daily administration (of the combination of Compound 1 and a nucleos(t)ide inhibitor described herein) the subject has sustained viral suppression. In some embodiments, the subject after 36 months of daily administration (of the combination of Compound 1 and a nucleos(t)ide inhibitor described herein) the subject has sustained viral suppression. In some embodiments, the subject after 42 months of daily administration (of the combination of Compound 1 and a nucleos(t)ide inhibitor described herein) the subject has sustained viral suppression. In some embodiments, the subject after 44 months of daily administration (of the combination of Compound 1 and a nucleos(t)ide inhibitor described herein) the subject has sustained viral suppression. In some embodiments, the subject after 48 months of daily administration (of the combination of Compound 1 and a nucleos(t)ide inhibitor described herein) the subject has sustained viral suppression. In some embodiments, the subject after 54 months of daily administration (of the combination of Compound 1 and a nucleos(t)ide inhibitor described herein) the subject has sustained viral suppression.

In some embodiments, after 2 weeks of daily administration (of the combination of Compound 1 and a nucleos(t)ide inhibitor described herein), the subject has a reduction in HBV DNA or HBV RNA levels. In some embodiments, after 4 weeks of daily administration (of the combination of Compound 1 and a nucleos(t)ide inhibitor described herein), the subject has a reduction in HBV DNA or HBV RNA levels. In some embodiments, after 8 weeks of daily administration (of the combination of Compound 1 and a nucleos(t)ide inhibitor described herein), the subject has a reduction in HBV DNA or HBV RNA levels. In some embodiments, after 12 weeks of daily administration (of the combination of Compound 1 and a nucleos(t)ide inhibitor described herein), the subject has a reduction in HBV DNA or HBV RNA levels. In some embodiments, after 24 weeks of daily administration (of the combination of Compound 1 and a nucleos(t)ide inhibitor described herein) the subject has a reduction in HBV DNA or HBV RNA. In some embodiments, after 30 weeks of daily administration (of the combination of Compound 1 and a nucleos(t)ide inhibitor described herein) the subject has a reduction in HBV DNA or HBV RNA. In some embodiments, the subject after 36 weeks of daily administration (of the combination of Compound 1 and a nucleos(t)ide inhibitor described herein) the subject has a reduction in HBV DNA or HBV RNA. In some embodiments, the subject after 44 weeks of daily administration (of the combination of Compound 1 and a nucleos(t)ide inhibitor described herein) the subject has a reduction in HBV DNA or HBV RNA. In some embodiments, the subject after 12 months of daily administration (of the combination of Compound 1 and a nucleos(t)ide inhibitor described herein) the subject has a reduction in HBV DNA or HBV RNA. In some embodiments, the subject after 18 months of daily administration (of the combination of Compound 1 and a nucleos(t)ide inhibitor described herein) the subject has a reduction in HBV DNA or HBV RNA. In some embodiments, the subject after 24 months of daily administration (of the combination of Compound 1 and a nucleos(t)ide inhibitor described herein) the subject has a reduction in HBV DNA or HBV RNA. In some embodiments, the subject after 30 months of daily administration (of the combination of Compound 1 and a nucleos(t)ide inhibitor described herein) the subject has a reduction in HBV DNA or HBV RNA. In some embodiments, the subject after 36 months of daily administration (of the combination of Compound 1 and a nucleos(t)ide inhibitor described herein) the subject has a reduction in HBV DNA or HBV RNA. In some embodiments, the subject has a HBV DNA reduction that is below the detectable limit using a PCR-assay. In some embodiments, the subject has a HBV RNA level that is below the limit of detection.

In some embodiments, after 2 weeks of daily administration (of the combination of Compound 1 and a nucleos(t)ide inhibitor described herein) the subject has greater than 0.5 log₁₀ decline in HBeAg. In some embodiments, after 4 weeks of daily administration (of the combination of Compound 1 and a nucleos(t)ide inhibitor described herein) the subject has greater than 0.5 log₁₀ decline in HBeAg. In some embodiments, after 8 weeks of daily administration (of the combination of Compound 1 and a nucleos(t)ide inhibitor described herein) the subject has greater than 0.5 log₁₀ decline in HBeAg. In some embodiments, after 12 weeks of daily administration (of the combination of Compound 1 and a nucleos(t)ide inhibitor described herein) the subject has greater than 0.5 log₁₀ decline in HBeAg. In some embodiments, after 16 weeks of daily administration (of the combination of Compound 1 and a nucleos(t)ide inhibitor described herein) the subject has greater than 0.5 log₁₀ decline in HBeAg. In some embodiments, after 18 weeks of daily administration (of the combination of Compound 1 and a nucleos(t)ide inhibitor described herein) the subject has greater than 0.5 log₁₀ decline in HBeAg. In some embodiments, after 24 weeks of daily administration (of the combination of Compound 1 and a nucleos(t)ide inhibitor described herein) the subject has greater than 0.5 log₁₀ decline in HBeAg. In some embodiments, after 30 weeks of daily administration (of the combination of Compound 1 and a nucleos(t)ide inhibitor described herein) the subject has greater than 0.5 log₁₀ decline in HBeAg. In some embodiments, after 36 weeks of daily administration (of the combination of Compound 1 and a nucleos(t)ide inhibitor described herein) the subject has greater than 0.5 log₁₀ decline in HBeAg. In some embodiments, after 44 weeks of daily administration (of the combination of Compound 1 and a nucleos(t)ide inhibitor described herein) the subject has greater than 0.5 log₁₀ decline in HBeAg. In some embodiments, after 12 months of daily administration (of the combination of Compound 1 and a nucleos(t)ide inhibitor described herein) the subject has greater than 0.5 log₁₀ decline in HBeAg. In some embodiments, after 18 months of daily administration (of the combination of Compound 1 and a nucleos(t)ide inhibitor described herein) the subject has greater than 0.5 log₁₀ decline in HBeAg. In some embodiments, after 24 months of daily administration (of the combination of Compound 1 and a nucleos(t)ide inhibitor described herein) the subject has greater than 0.5 log₁₀ decline in HBeAg. In some embodiments, after 30 months of daily administration (of the combination of Compound 1 and a nucleos(t)ide inhibitor described herein) the subject has greater than 0.5 log₁₀ decline in HBeAg. In some embodiments, after 36 months of daily administration (of the combination of Compound 1 and a nucleos(t)ide inhibitor described herein) the subject has greater than 0.5 log₁₀ decline in HBeAg. In some embodiments, after 42 months of daily administration (of the combination of Compound 1 and a nucleos(t)ide inhibitor described herein) the subject has greater than 0.5 log₁₀ decline in HBeAg. In some embodiments, after 44 months of daily administration (of the combination of Compound 1 and a nucleos(t)ide inhibitor described herein) the subject has greater than 0.5 log₁₀ decline in HBeAg. In some embodiments, after 50 months of daily administration (of the combination of Compound 1 and a nucleos(t)ide inhibitor described herein) the subject has greater than 0.5 log₁₀ decline in HBeAg.

In some embodiments, after 2 weeks of daily administration (of the combination of Compound 1 and a nucleos(t)ide inhibitor described herein) the subject has hepatitis B virus levels that are below detection levels in the subject. In some embodiments, after 4 weeks of daily administration (of the combination of Compound 1 and a nucleos(t)ide inhibitor described herein) the subject has hepatitis B virus levels that are below detection levels in the subject. In some embodiments, after 8 weeks of daily administration (of the combination of Compound 1 and a nucleos(t)ide inhibitor described herein) the subject has hepatitis B virus levels that are below detection levels in the subject. In some embodiments, after 18 weeks of daily administration (of the combination of Compound 1 and a nucleos(t)ide inhibitor described herein) the subject has hepatitis B virus levels that are below detection levels in the subject. In some embodiments, after 24 weeks of daily administration (of the combination of Compound 1 and a nucleos(t)ide inhibitor described herein) the subject has hepatitis B virus levels that are below detection levels in the subject. In some embodiments, after 30 weeks of daily administration (of the combination of Compound 1 and a nucleos(t)ide inhibitor described herein) the subject has hepatitis B virus levels that are below detection levels in the subject. In some embodiments, after 36 weeks of daily administration (of the combination of Compound 1 and a nucleos(t)ide inhibitor described herein) the subject has hepatitis B virus levels that are below detection levels in the subject. In some embodiments, after 42 weeks of daily administration (of the combination of Compound 1 and a nucleos(t)ide inhibitor described herein) the subject has hepatitis B virus levels that are below detection levels in the subject. In some embodiments, after 44 weeks of daily administration (of the combination of Compound 1 and a nucleos(t)ide inhibitor described herein) the subject has hepatitis B virus levels that are below detection levels in the subject. In some embodiments, after 12 months of daily administration (of the combination of Compound 1 and a nucleos(t)ide inhibitor described herein) the subject has hepatitis B virus levels that are below detection levels in the subject. In some embodiments, after 18 months of daily administration (of the combination of Compound 1 and a nucleos(t)ide inhibitor described herein) the subject has hepatitis B virus levels that are below detection levels in the subject. In some embodiments, after 24 months of daily administration (of the combination of Compound 1 and a nucleos(t)ide inhibitor described herein) the subject has hepatitis B virus levels that are below detection levels in the subject. In some embodiments, after 30 months of daily administration (of the combination of Compound 1 and a nucleos(t)ide inhibitor described herein) the subject has hepatitis B virus levels that are below detection levels in the subject. In some embodiments, after 36 months of daily administration (of the combination of Compound 1 and a nucleos(t)ide inhibitor described herein) the subject has hepatitis B virus levels that are below detection levels in the subject. In some embodiments, after 42 months of daily administration (of the combination of Compound 1 and a nucleos(t)ide inhibitor described herein) the subject has hepatitis B virus levels that are below detection levels in the subject. In some embodiments, after 44 months of daily administration (of the combination of Compound 1 and a nucleos(t)ide inhibitor described herein) the subject has hepatitis B virus levels that are below detection levels in the subject. In some embodiments, after 50 months of daily administration (of the combination of Compound 1 and a nucleos(t)ide inhibitor described herein) the subject has hepatitis B virus levels that are below detection levels in the subject.

### Stopping Criteria

In some aspects, methods described herein have a stopping criteria, or a criteria that if met, the subject treated for hepatitis B is no longer administered Compound 1 and a nucleos(t)ide inhibitor, such as entecavir, tenofovir or tenofovir alafenamide fumarate, in combination. In some embodiments, the subject is virologically suppressed and HBeAg negative before administering the compound or the combination therapy. In other aspects, the subject is virologically suppressed and HBeAg positive before administering the compound or the combination therapy. In other embodiments, the subject is treatment naïve and HBeAg positive before administering the compound or the combination therapy. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein.

Subjects are administered daily an amount of the compound, i.e., Compound 1, for example about 200 mg to about 400 mg, or about 250 mg to about 350 mg, or about 300 mg, and a nucleos(t)ide inhibitor for about 12 weeks, for about 18 weeks, for about 24 weeks, for about 28 weeks, for about 32 weeks, for about 36 weeks, for about 38 weeks, for about 40 weeks, for about 42 weeks, for about 44 weeks, for about 50 weeks, for about 56 weeks, for about 60 weeks, for about 64 weeks, for about 68 weeks, for about 70 weeks, for about 72 weeks, for about 74 weeks, for about 76 weeks, for about 78 weeks, for about 80 weeks, or for about 84 weeks. Compound 1 is administered in a pharmaceutical composition disclosed herein, for example, in a spray dried dispersion. In some embodiments, Compound 1 is administered in a form as described in the Examples, for example in Example 5 herein. In some embodiments, the compound is administered to the patient in a solid dosage form as described in the Examples, for example, Examples 1-5 herein. In some embodiments, 300 mg of the compound is administered to the patient in a solid dosage form as described in the Examples, for example, Examples 1-5 herein. Subjects are assessed, for example, for hepatitis B viral DNA and HBeAg while receiving the combination therapy of Compound 1 and the nucleos(t)ide inhibitor. If after about 42 weeks, about 44 weeks, about 50 weeks, about 56 weeks, about 60 weeks, about 64 weeks, about 68 weeks, about 70 weeks, about 72 weeks, about 74 weeks, about 76 weeks, about 78 weeks, about 80 weeks, or about 84 weeks the subject meets the stopping criteria, then administration of Compound 1 and the nucleos(t)ide inhibitor is stopped.

In some embodiments, the stopping criteria is having a hepatitis B viral DNA concentration of less than 20 IU/mL and a HBeAg concentration of less than or equal to 5 IU/mL. In other aspects, the stopping criteria is having a hepatitis B viral DNA concentration of less than 20 IU/mL and HBeAg negative.

For example, if a subject that was virologically suppressed and HBeAg negative before administration of the compound, or the combination therapy, has a hepatitis B viral DNA concentration of less than 20 IU/mL and a HBeAg concentration of less than or equal to 5 IU/mL for at least six months prior to the 76′ week of administering the compound, administration of the compound and the nucleos(t)ide inhibitor is stopped. If a subject that was virologically suppressed and HBeAg negative before administration of the compound, or the combination therapy, has a hepatitis B viral DNA concentration of less than 20 IU/mL and a HBeAg concentration of less than or equal to 5 IU/mL for at least four months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. If a subject that was virologically suppressed and HBeAg negative before administration of the compound, or the combination therapy, has a hepatitis B viral DNA concentration of less than 20 IU/mL and a HBeAg concentration of less than or equal to 5 IU/mL for at least three months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. If a subject that was virologically suppressed and HBeAg negative before administration of the compound, or the combination therapy, has a hepatitis B viral DNA concentration of less than 20 IU/mL and a HBeAg concentration of less than or equal to 5 IU/mL for at least eight months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. After administration of Compound 1 and the nucleos(t)ide is stopped, the subject is monitored for up to three years for hepatitis B viral DNA concentration and HBeAg concentration.

In some embodiments, subjects are administered Compound 1 and the nucleos(t)ide inhibitor in combination for a treatment period, e.g. 76 weeks. In some embodiments, subjects are administered a placebo and the nucleos(t)ide inhibitor for an initial period, e.g. 24 weeks, and are then administered Compound 1 and the nucleos(t)ide inhibitor in combination for several weeks in a treatment period, e.g. weeks 24-weeks 76. Subjects receiving Compound 1 and the nucleos(t)ide inhibitor in combination initially and subjects receiving a placebo and the nucleos(t)ide inhibitor initially are both assessed the stopping criteria at the end of the treatment period, e.g. week 76.

For example, if a subject that was virologically suppressed and HBeAg positive before administration of the compound, or the combination therapy, has a hepatitis B viral DNA concentration of less than 20 IU/mL and a HBeAg concentration of less than or equal to 5 IU/mL for at least six months prior to the 76^{th} week of administering the compound, administration of the compound and the nucleos(t)ide inhibitor is stopped. If a subject that was virologically suppressed and HBeAg positive before administration of the compound, or the combination therapy, has a hepatitis B viral DNA concentration of less than 20 IU/mL and a HBeAg concentration of less than or equal to 5 IU/mL for at least four months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. If a subject that was virologically suppressed and HBeAg positive before administration of the compound, or the combination therapy, has a hepatitis B viral DNA concentration of less than 20 IU/mL and a HBeAg concentration of less than or equal to 5 IU/mL for at least three months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. If a subject that was virologically suppressed and HBeAg positive before administration of the compound, or the combination therapy, has a hepatitis B viral DNA concentration of less than 20 IU/mL and a HBeAg concentration of less than or equal to 5 IU/mL for at least eight months prior to the 52^{nd}, 72^{nd}, 74^{th}, 76^{th}, 78^{th}, 80^{th}, or 82^{nd} week of administering the compound, administration of the compound and the nucleos(t)ide inhibitor is stopped. If, the virologically suppressed and HBeAg positive subject has a hepatitis B viral DNA concentration of greater than or equal to 20 IU/mL or a HBeAg concentration of greater than 5 IU/mL during the six months prior to the 76^{th} week of administering the compound, administration of the compound is stopped and administration of the nucleos(t)ide inhibitor is continued. If, the virologically suppressed and HBeAg positive subject has a hepatitis B viral DNA concentration of greater than or equal to 20 IU/mL or a HBeAg concentration of greater than 5 IU/mL during the six months prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor is continued. If, the virologically suppressed and HBeAg positive subject has a hepatitis B viral DNA concentration of greater than or equal to 20 IU/mL or a HBeAg concentration of greater than 5 IU/mL during the three months prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor is continued. If, the virologically suppressed and HBeAg positive subject has a hepatitis B viral DNA concentration of greater than or equal to 20 IU/mL or a HBeAg concentration of greater than 5 IU/mL during the four months prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor is continued. If, the virologically suppressed and HBeAg positive subject has a hepatitis B viral DNA concentration of greater than or equal to 20 IU/mL or a HBeAg concentration of greater than 5 IU/mL during the three months prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor is continued. If, the virologically suppressed and HBeAg positive subject has a hepatitis B viral DNA concentration of greater than or equal to 20 IU/mL or a HBeAg concentration of greater than 5 IU/mL during the eight months prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor is continued. If the subject meets the stopping criteria, the subject is monitored for up to three years after administering the compound has stopped for hepatitis B viral DNA concentration and HBeAg concentration. If the subject does not meet the stopping criteria, the subject is monitored for up to twelve weeks for hepatitis B viral DNA concentration and HBeAg concentration.

For a subject is treatment naïve and HBeAg positive before the subject started the combination therapy of Compound 1 and a nucleos(t)ide, if after 76 weeks of administering the compound (i.e. Compound 1) and the nucleosi(t)e the subject has a pgRNA decline of greater than or equal to 2.5 log₁₀ U/mL from baseline s prior to the 76^{th} week of administering the compound, administration of the compound and nucleos(t)ide inhibitor continues up to 48 weeks. If the subject has a pgRNA decline of less than 2.5 log₁₀ U/mL from prior to the 76^{th} week of administering the compound, administration of the compound is stopped and administration of the nucleos(t)ide inhibitor continues.

For a subject is treatment naïve and HBeAg positive before the subject started the combination therapy of Compound 1 and a nucleos(t)ide, if after 52^{nd}, 72^{nd}, 74^{th}, 76^{th}, 78^{th}, 80^{th}, or 82^{nd} weeks of administering the compound (i.e. Compound 1) and the nucleosi(t)e the subject has a pgRNA decline of greater than or equal to 2.5 log₁₀ U/mL from baseline, administration of the compound and nucleos(t)ide inhibitor continues up to 48 weeks. If the subject has a pgRNA decline of less than 2.5 log₁₀ U/mL from baseline prior to the 52^{nd}, 72^{nd}, 74^{th}, 76^{th}, 78^{th}, 80^{th}, or 82^{nd} week of administering the compound, administration of the compound is stopped and administration of the nucleos(t)ide inhibitor continues.

In some embodiments, the compound is administered to the patient in a solid dosage form as described in the Examples, for example, Examples 1-5 herein, pursuant to the methods described herein, for example, administering 300 mg of Compound 1 and administering a nucleos(t)ide inhibitor for the treatment of HBV in a subject in need thereof.

In some embodiments, the compound is administered to the patient in a solid dosage form as described in the Examples, for example, Examples 1-5 herein, pursuant to the methods described herein, for example, administering about 250 mg to about 350 mg of Compound 1 and administering a nucleos(t)ide inhibitor for the treatment of HBV in a subject in need thereof.

For use in accordance with this aspect, the appropriate dosage is expected to vary depending on, for example, the particular compound employed, the mode of administration, and the nature and severity of the infection to be treated as well as the specific infection to be treated and is within the purview of the treating physician. Usually, an indicated administration dose may be in the range between about 0.1 to about 1000 μg/kg body weight. In some cases, the administration dose of the compound may be less than 400 μg/kg body weight. In other cases, the administration dose may be less than 200 μg/kg body weight. In yet other cases, the administration dose may be in the range between about 0.1 to about 100 μg/kg body weight. The dose may be conveniently administered once daily, or in divided doses up to, for example, four times a day or in sustained release form.

A pharmaceutical composition of the present disclosure may be administered by any conventional route, in particular: enterally, topically, orally, nasally, e.g. in the form of tablets or capsules, via suppositories, or parenterally, e.g. in the form of injectable solutions or suspensions, for intravenous, intra-muscular, sub-cutaneous, or intra-peritoneal injection. Suitable pharmaceutical compositions will include those formulated in a conventional manner using one or more physiologically acceptable carriers or excipients, and any of those known and commercially available and currently employed in the clinical setting. Thus, the pharmaceutical compositions may be formulated for oral, buccal, topical, parenteral, rectal or transdermal administration or in a form suitable for administration by inhalation or insufflation (either orally or nasally).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). Preparations may also contain buffer salts, flavoring, coloring and sweetening agents as appropriate.

Pharmaceutical compositions for oral administration may also be suitably formulated to give controlled-release or sustained release of the active compound(s) over an extended period. For buccal administration, the compositions may take the form of tablets or lozenges formulated in a conventional manner known to the skilled artisan.

Pharmaceutical compositions may also be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the compositions may be in powder form for constitution with a suitable vehicle, e.g. sterile pyrogen-free water, before use. Pharmaceutical compositions may also be formulated for rectal administration as suppositories or retention enemas, e.g. containing conventional suppository bases such as cocoa butter or other glycerides. Also contemplated herein are methods that include administering a second active agent both independently and via compositions that include a second active agent. For example, in addition to being infected with HBV, a subject or patient can further have HBV infection-related co-morbidities, i.e., diseases and other adverse health conditions associated with, exacerbated by, or precipitated by being infected with HBV. Contemplated herein are disclosed pharmaceutical compositions in combination with at least one other agent that has previously been shown to treat these HBV-infection-related conditions.

In some cases, a disclosed pharmaceutical composition may be administered as part of a combination therapy in conjunction with one or more antivirals, including nucleoside analogs, and other assembly effectors, for instance heteroaryldihydropyrimidines (HAPs) such as methyl 4-(2-chloro-4-fluorophenyl)-6-methyl-2-(pyridin-2-yl)-1,4-dihydropyrimidine-5-carboxylate (HAP-1). For example, provided herein is a method of treating a patient suffering from hepatitis B infection comprising administering to the patient a first amount of Compound 1 and a second amount of an antiviral, or other anti HBV agent, for example a second amount of a second compound selected from the group consisting of: a HBV capsid assembly promoter (for example, GLS4, BAY 41-4109, AT-130, DVR-23 (e.g., as depicted below),

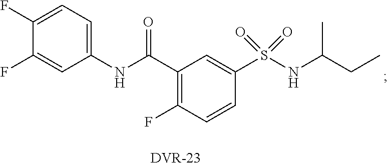

NVR 3-778, NVR1221 (by code); and N890 (as depicted below):

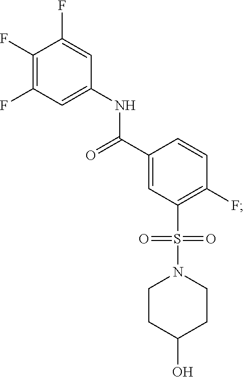 [SMILES: O=C(Nc1cc(F)c(F)c(F)c1)c1ccc(F)c(S(=O)(=O)N2CCC(O)CC2)c1]

other CpAMs such as those disclosed in the following patent applications hereby incorporated by reference: WO2014037480, WO2014184328, WO2013006394, WO2014089296, WO2014106019, WO2013102655, WO2014184350, WO2014184365, WO2014161888, WO2014131847, WO2014033176, WO2014033167, and WO2014033170; Nucleoside analogs interfering with viral polymerase, such as entecavir (Baraclude), Lamivudine, (Epivir-HBV), Telbivudine (Tyzeka, Sebivo), Adefovir dipivoxil (Hepsera), Tenofovir (Viread), Tenofovir alafenamide fumarate (TAF), prodrugs of tenofavir (e.g. AGX-1009), L-FMAU (Clevudine), LB80380 (Besifovir) and:

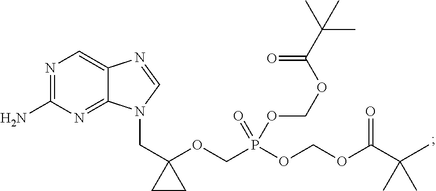 [SMILES: CC(C)(C)C(=O)OCOP(=O)(COC1(Cn2cnc3cnc(N)nc32)CC1)OCOC(=O)C(C)(C)C]

viral entry inhibitors such as Myrcludex B and related lipopeptide derivatives; HBsAg secretion inhibitors such as REP 9AC′ and related nucleic acid-based amphipathic polymers, HBF-0529 (PBHBV-001), PBHBV-2-15 as depicted below:

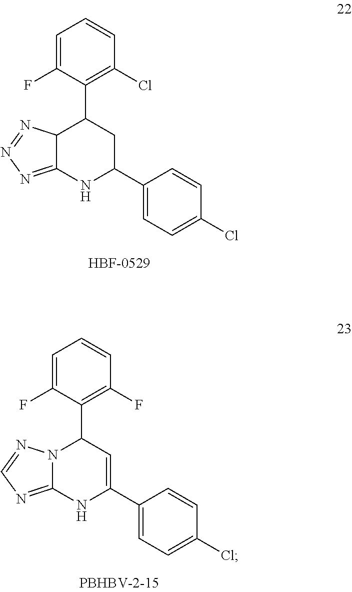 [SMILES: Fc1cccc(Cl)c1C1CC(c2ccc(Cl)cc2)NC2=NN=NC21 ; Fc1cccc(F)c1C1C=C(c2ccc(Cl)cc2)Nc2ncnn21]

and BM601 as depicted below:

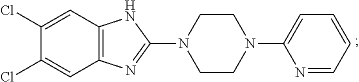 [SMILES: Clc1cc2nc(N3CCN(c4ccccn4)CC3)[nH]c2cc1Cl]

disruptors of nucleocapsid formation or integrity such as NZ-4/W28F:

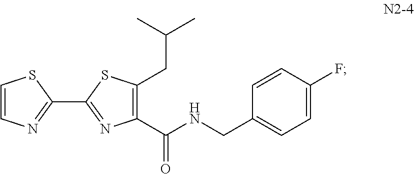 [SMILES: CC(C)Cc1sc(-c2nccs2)nc1C(=O)NCc1ccc(F)cc1]

cccDNA formation inhibitors such as BSBI-25, CCC-0346, CCC-0975 (as depicted below):

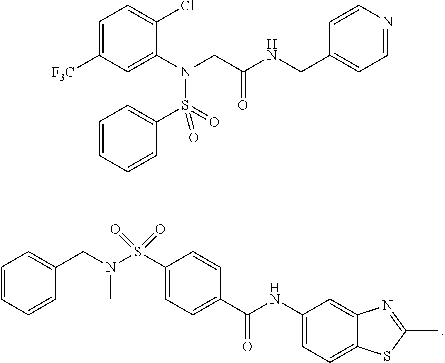 [SMILES: O=C(CN(c1cc(C(F)(F)F)ccc1Cl)S(=O)(=O)c1ccccc1)NCc1ccncc1 ; Cc1nc2cc(NC(=O)c3ccc(S(=O)(=O)N(C)Cc4ccccc4)cc3)ccc2s1]

HBc directed transbodies such as those described in Wang Y, et al, Transbody against hepatitis B virus core protein inhibits hepatitis B virus replication in vitro, Int. Immunopharmacol (2014), located at //dx.doi.org/10.1016/j.intimp.2015.01.028; antiviral core protein mutant (such as Cp183-V124W and related mutations as described in WO/2013/010069, WO2014/074906, each incorporated by reference); inhibitors of HBx-interactions such as RNAi, antisense and nucleic acid based polymers targeting HBV RNA, e.g., RNAi (for example ALN-HBV, ARC-520, TKM-HBV, ddRNAi), antisense (ISIS-HBV), or nucleic acid based polymer: (REP 2139-Ca); Pegylated IFN 2b, IFN lambda 1a and PEG IFN lambda 1a, Wellferon, Infergen, lymphotoxin beta agonists such as CBE11 and BS1); Non-Interferon Immune enhancers such as Thymosin alpha-1 (Zadaxin) and Interleukin-7 (CYT107); TLR-7/9 agonists such as GS-9620, CYT003, Resiquimod; Cyclophilin Inhibitors such as NVPO18; OCB-030; SCY-635; Alisporivir; NIM811 and related cyclosporine analogs; vaccines such as GS-4774, TG1050, Core antigen vaccine; SMAC mimetics such as birinapant and other IAP-antagonists; Epigenetic modulators such as KMT inhibitors (EZH1/2, G9a, SETD7, Suv39 inhibitors), PRMT inhibitors, HDAC inhibitors, SIRT agonists, HAT inhibitors, WD antagonists (e.g. OICR-9429), PARP inhibitors, APE inhibitors, DNMT inhibitors, LSD1 inhibitors, JMJD HDM inhibitors, and Bromodomain antagonists; kinase inhibitors such as TKB1 antagonists, PLK1 inhibitors, SRPK inhibitors, CDK2 inhibitors, ATM & ATR kinase inhibitors; STING Agonists; Ribavirin; N-acetyl cysteine; NOV-205 (BAM205); Nitazoxanide (Alinia), Tizoxanide; SB 9200 Small Molecule Nucleic Acid Hybrid (SMNH); DV-601; Arbidol; FXR agonists (such as GW 4064 and Fexaramin); antibodies, therapeutic proteins, gene therapy, and biologics directed against viral components or interacting host proteins.

In some embodiments, the disclosure provides a method of treating a hepatitis B infection in a patient in need thereof, comprising: administering a pharmaceutical composition comprising Compound 1, or a pharmaceutically acceptable salt thereof, and one or more other HBV agents each selected from the group consisting of HBV capsid assembly promoters, HBF viral polymerase interfering nucleosides, viral entry inhibitors, HBsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a hepatitis B infection in a patient in need thereof, comprising: administering a first amount of a disclosed pharmaceutical composition comprising Compound 1, and administering a second amount of a HBV capsid assembly promoter.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of Compound 1 first, for example one or more days or weeks prior to initiation of administration of the antiviral. Moreover, additional drugs may be given in conjunction with the above combination therapy.

In another embodiment, Compound 1 may be conjugated (e.g., covalently bound directly or through molecular linker to a free carbon, nitrogen (e.g. an amino group), or oxygen (e.g. an active ester) of a disclosed compound), with a detection moiety, for e.g., a fluorophore moiety (such a moiety may for example re-emit a certain light frequency upon binding to a virus and/or upon photon excitation). Contemplated fluorophores include AlexaFluor® 488 (Invitrogen) and BODIPY FL (Invitrogen), as well as fluorescein, rhodamine, cyanine, indocarbocyanine, anthraquinones, fluorescent proteins, aminocoumarin, methoxycoumarin, hydroxycoumarin, Cy2, Cy3, and the like. Such disclosed compounds conjugated to a detection moiety may be used in e.g. a method for detecting HBV or biological pathways of HBV infection, e.g., in vitro or in vivo; and/or methods of assessing new compounds for biological activity.

### EXAMPLES

In order that the invention described herein can be more fully understood, the following examples are set forth. It should be understood that these examples are for illustrative purposes only and are not to be construed as limiting the invention in any manner.

### Example 1: Spray-Dried Dispersion (SDD) Formulations

Four Compound 1-polymer dispersion formulations were prepared as indicated in Table 1. Compound 1 and a polymer were dissolved in acetone/H₂O (95/5). Each formulation was spray dried from acetone/H₂O (95/5) at a 20:80 Compound 1: polymer weight ratio. A Buchi B290 spray dryer was used at a high efficiency (standard) cyclone (closed loop configuration; 0.7 mm liquid cap; and 1.5 mm gas cap). The spray solution pump set point was 22 g/min (±4), the spray solution atomization pressure was 28 psi (±5), the inlet drying gas temperature was 42° C. (±5), the drying gas flow rate was 100% (Aspirator Set Point), and the condenser outlet temperature was −20° C. (±6). The process included a secondary drying step using a convection tray dryer (Despatch 4 or 14 ft³), with a drying temperature set point of 40° C., a bed depth of approximately 1 inch, and a total drying time of at least 24 hours. The batch size of total solids was 2.5 g and the solution composition was 8% solids.

The four formulations of Compound 1 prepared above are listed in Table 1 with the dry yield percentage and T_{g}.

TABLE 1

| | Dry | Measure T_{g} | |
|---|---|---|---|
| Formulation | Yield | (° C.) | Polymer |
| 1 | 88% | 178 | Poly(methacrylic acid-co-methyl |
| | | | methacrylate)(1:1)¹ |
| 2 | 87% | 105 | Hypromellose Acetate Succinate |
| | | | MG grade(HPMCAS-M)² |
| 3 | 88% | 107 | Hypromellose Acetate Succinate |
| | | | HG grade(HPMCAS-H)³ |
| 4 | 84% | 126 | Hydroxypropyl Methylcellulose |
| | | | Phthalate(HPMCP HP-55)⁴ |

¹EUDRAGIT ® L 100 manufactured by Evonik
²AQOAT ®-MG manufactured by Shin Etsu
³AQOAT ®-HG manufactured by Shin Etsu
⁴HPMCP HP-55 manufactured by Shin Etsu

Compound 1 and the four spray-dried solid dispersions (SDDs) were characterized using modulated differential scanning calorimetry (MSDC), powder X-ray diffraction (PXRD), scanning electron microscopy (SEM), and non-sink dissolution, as discussed below.

a. Modulated Differential Scanning Calorimetry (MDSC) Analysis

Modulated differential Scanning calorimetry (MDSC) was performed using a TA Instruments Q2000 differential scanning calorimeter equipped with a TA instruments Refrigerated Cooling System 90. MDSC was used to measure glass transition temperature (T_{g}), cold crystallization temperature (T_{c}), defined as a crystallization event at a temperature lower than the melting temperature, and melting temperature (Tₘ).

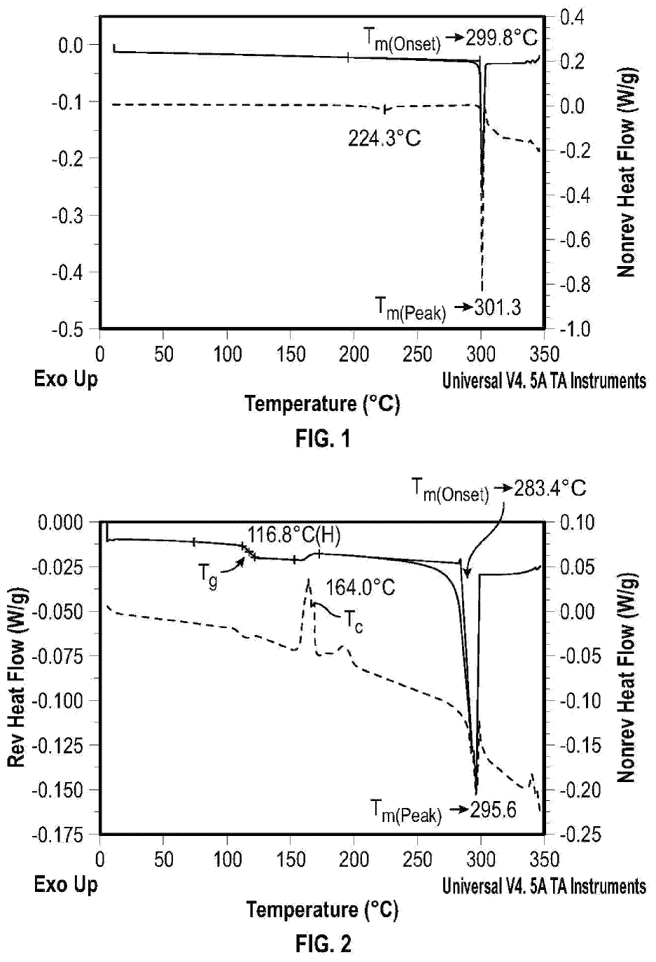

For Compound 1, the T_{g} was measured via a melt-quench technique, heating past its melting temperature and rapidly cooling to trap the molten material in an amorphous state. The resulting sample was analyzed and found to have a T_{g} of 117° C. and a T_{c} of 164.0° C. A melt event was observed at 301° C. A Tₘ/T_{g} ratio (K/K) of 1.47 was determined. The Tₘ/T_{g} ratio is a strong indicator of a molecule's crystal lattice energy and its propensity to recrystallize. The thermographs for crystalline Compound 1 are shown in FIGS. 1 and 2.

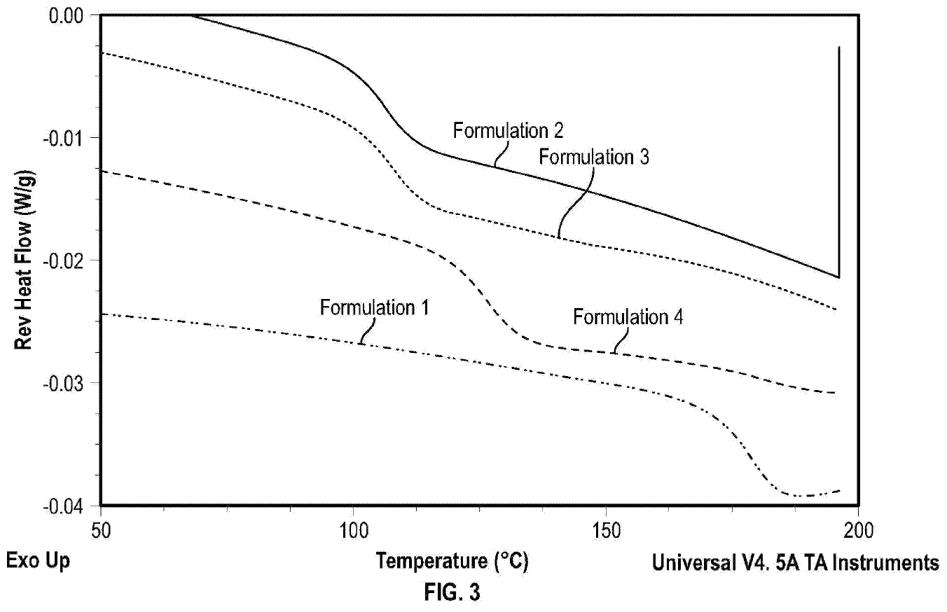

Thermal analysis of the four dispersion formulations (i.e., Formulations 1-4 prepared in Example 1, listed in Table 1) are shown in FIG. 3. All dispersions were found to have a single T_{g} indicating an intimately mixed amorphous solid dispersion with good homogeneity. The T_{g} is an indication of physical stability, indicating that the propensity of the API to recrystallize during long-term storage is low.

b. Powder X-Ray Diffraction (PXRD) Analysis

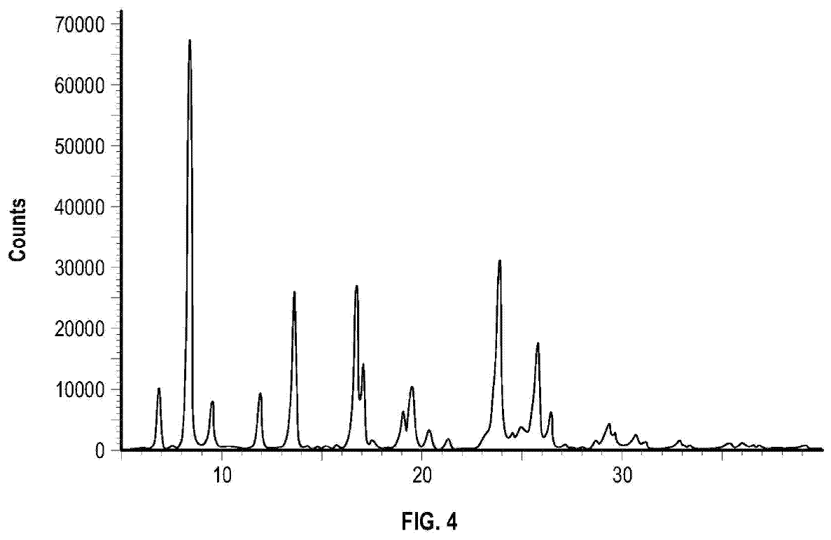

PXRD was performed using a Bruker D2 Phaser X-ray diffractometer to evaluate the crystallinity of spray dried formulations. Amorphous materials show an amorphous halo diffraction pattern, absent the discrete peaks that would be found in a crystalline material. The diffraction pattern of crystalline Compound 1 is shown in FIG. 4.

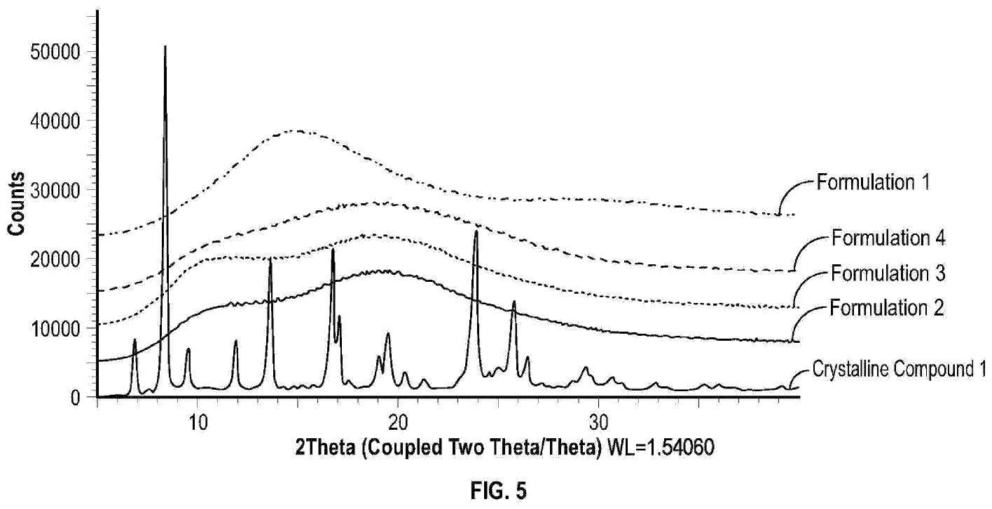

PXRD Diffractograms of Formulations 1-4 are shown in FIG. 5. Characterization by PXRD indicates that the SDDs are amorphous dispersions because no crystalline peaks were observed in the SDD diffractograms.

c. Scanning Electron Microscopy (SEM) Analysis

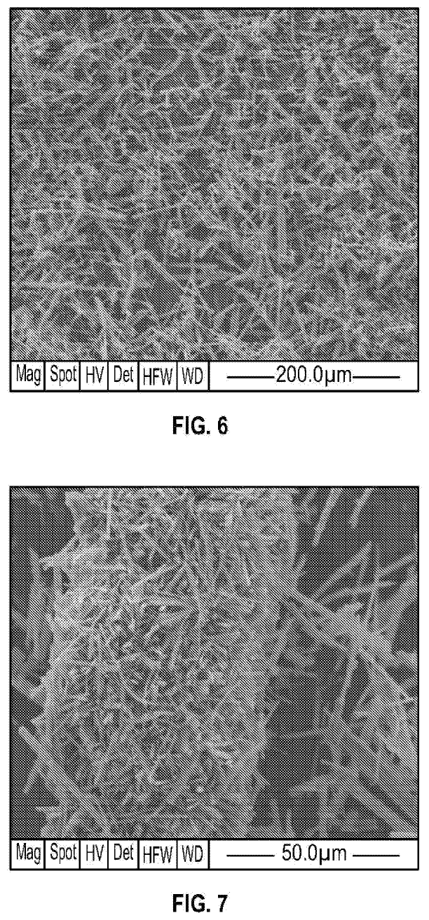

SEM samples were prepared by dispersing a testing sample (i.e., the SDD particles or crystalline Compound 1) onto an adhesive carbon-coated sample stub a coating with a thin conductive layer of gold using Polaron Autocoater E5200. Samples were analyzed using a FEI Quanta 200 SEM fitted with an Everhart-Thornley (second electron) detector, operating in high vacuum mode. Micrographs at various magnifications were captured for qualitative surface particle morphology analysis. SEM images of crystalline Compound 1 are shown in FIGS. 6 and 7.

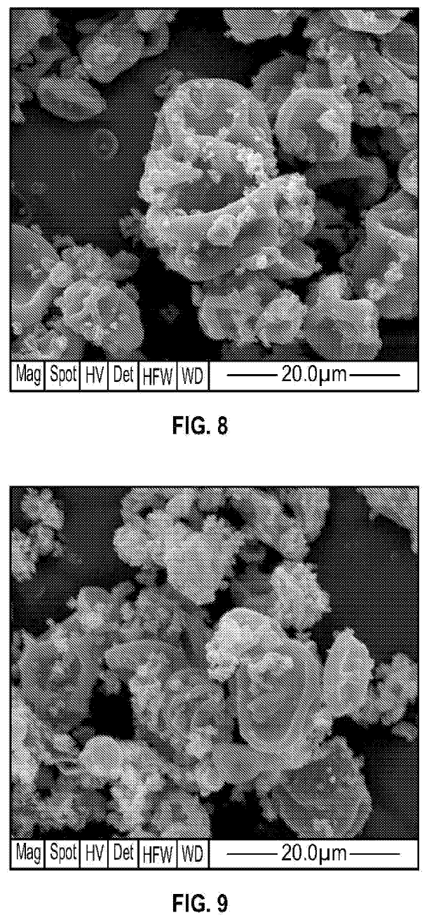

FIGS. 8-11 show the SEM images of the SDD particles, at 5,000× magnification, of the four formulations disclosed in Example 1 (see Table 1). FIG. 8 shows Formulation 2; FIG. 9 shows Formulation 3; FIG. 10 shows Formulation 1; and FIG. 11 shows Formulation 4. Typical SDD morphology is observed consisting of whole and collapsed spheres with smooth surfaces. No crystalline material was observed in any of Formulations 1-4.

d. Non-Sink Dissolution Analysis

In-vitro drug dissolution performance for each SDD prepared in Example 1 and described in Table 1 (i.e., Formulations 1-4) was evaluated by a two-stage gastric transfer non-sink dissolution test, which stimulates pH and bile salt concentrations for both gastric and intestinal exposure. A drug product sample start in 0.1N HCl_{(aq)} (simulated gastric fluid or SGF) for 30 minutes at which point an equal volume of concentrated fasted-state simulated intestinal fluid (FaSSIF) is added to the SGF, resulting in a final pH of 6.8 in FaSSIF (100 mM PBS, 2.24 mg/mL SIF, Biorelevant Inc.).

The dissolution performance of the SDDs of Formulations 1-4 and crystalline Compound 1 was tested. The dissolution test is used to measure the supersaturation of drug above the bulk crystalline Compound 1 solubility in biorelevant intestinal media (FaSSIF) after 30 minutes exposure to a low-pH environment (SGF). During the test, samples were transferred from SGF [theoretical Cₘₐₓ=1000 μA/mL] to FaSSIF SGF [theoretical Cₘₐₓ=500 μA/mL]. The results are reported in Table 2.

TABLE 2

| | C_{max FaSSIF}^{a} | C₂₁₀^{b} | AUC_{35-210FaSSIF}^{c} | |
|---|---|---|---|---|
| Formulation | (μgA/mL) | (μgA/mL) | (min * μgA/mL) | |
| 1 | 207.9 | 35.1 | 11600 | 20:80 Compound 1:Poly(methacrylic |
| | | | | acid-co-methyl methacrylate)(1:1) |
| | | | | (Eudragit L 100) |
| 2 | 182.2 | 182.2 | 28200 | 20:80 Compound 1:Hypromellose |
| | | | | Acetate Succinate MG grade |
| | | | | (HPMCAS-M) |
| 3 | 123.5 | 123.1 | 21400 | 20:80 Compound 1:Hypromellose |
| | | | | Acetate Succinate HG grade |
| | | | | (HPMCAS-H) |
| 4 | 201.4 | 28.6 | 10400 | 20:80 Compound 1:Hydroxypropyl |
| | | | | Methylcellulose Phthalate |
| | | | | (HPMCP HP-55) |
| 5 | 33.4 | 27.6 | 5300 | Compound 1 Bulk Crystalline API |

^{a}C_{max FaSSIF} = maximum drug concentration after transfer to FaSSIF
^{b}C₂₁₀ = drug concentration at 180 minutes after transfer to FaSSIF
^{c}AUC_{35-210FaSSIF} = area under the curve after transfer to FaSSIF from 35 to 210 minutes

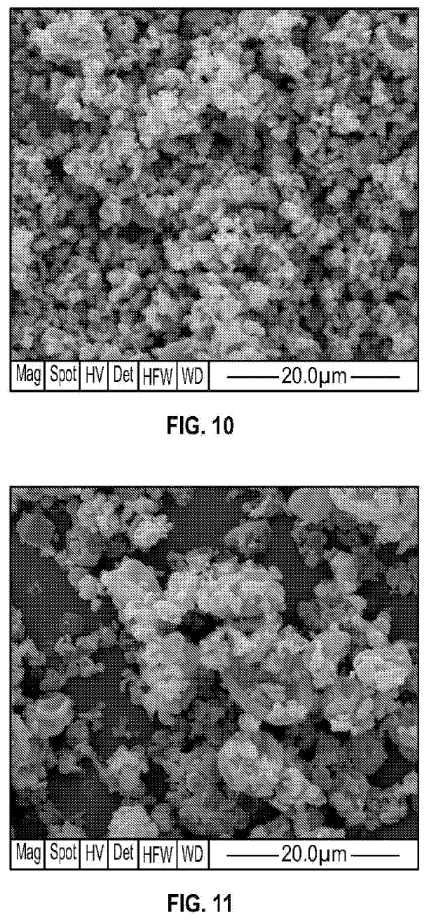

The SGF/FaSSIF non-sink dissolution test for SDDs of Formulations 1-4 compared to bulk crystalline Compound 1 are shown in FIG. 12. Formulations 2 and 3 provided a 4 to 5-fold enhancement for solubilized drug compared to bulk crystalline drug (AUC_{SDD}/AUC_{API}).

e. Suspension Stability

Suspension stability for SDDs of Formulation 2 and Formulation 4 were evaluated with suspension concentrations of 40 mg active (200 mg SDD) per 1 mL of 0.5 wt % METHOCEL A4M® (methyl cellulose, commercially available from Sigma Aldrich). The SDDs were prepared as disclosed in Example 1 with a 20:80 Compound 1: polymer ratio. The performance of the SDD suspensions were monitored after 4 hours using the SGF/FaSSIF dissolution. The SDD suspensions were dosed 4 hours after preparation.

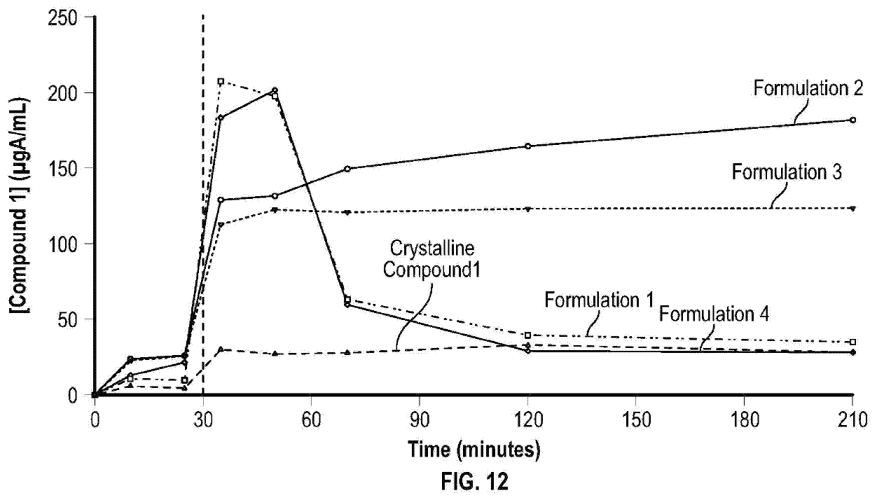

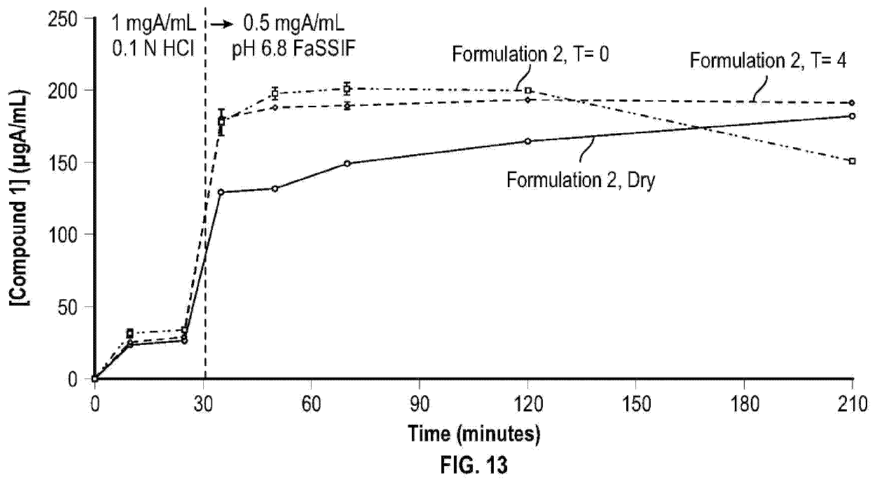

Stable dissolution performance through 4 hours (when stored at room temperature 21° C. with 100 rpm stirring) was observed for SDDs of Formulation 2 and Formulation 4, which would allow sufficient time for in vivo dosing following constitution of the suspensions. The suspension stability results for the tested SDD formulations can be found in FIG. 13 and Table 3; and FIG. 14 and Table 4.

FIG. 13 shows the SGF/FaSSIF non-sink dissolution test results for the SDD of prepared as a suspension in 0.5% METHOCEL A4M® (methyl cellulose, commercially available from Sigma Aldrich), dosed 4 hours after constitution compared to the SDD of Formulation 2 dosed as dry powder. See also Table 3.

TABLE 3
Formulation 2

| C_{max FaSSIF} | C₂₁₀ | AUC_{35-210FaSSIF} | |
|---|---|---|---|
| (μgA/mL) | (μgA/mL) | (min * μgA/mL) | State |
| 182.2 | 182.2 | 28200 | Formulation 2 Dry Power |
| 200.8 | 151.2 | 32600 | Formulation 2 in Suspension |
| | | | (Initial) |
| 193.2 | 191.1 | 33400 | Formulation 2 in Suspension |
| | | | (4 Hrs.) |

FIG. 14 shows the SGF/FaSSIF non-sink dissolution test results for the SDD of Formulation 4 prepared as a suspension in 0.5% METHOCEL A4M® (methyl cellulose, commercially available from Sigma Aldrich), dosed 4 hours after constitution compared to the SDD of Formulation 4 dosed as dry powder. See also Table 4.

TABLE 4
Formulation 4

| C_{max FaSSIF} | C₂₁₀ | AUC_{35-210FaSSIF} | |
|---|---|---|---|
| (μgA/mL) | (μgA/mL) | (min * μgA/mL) | State |
| 201.4 | 28.6 | 10400 | Formulation 4 Dry Power |
| 326.0 | 35.1 | 23800 | Formulation 4 in Suspension |
| | | | (Initial) |
| 348.7 | 53.2 | 29000 | Formulation 4 in Suspension |
| | | | (4 Hrs.) |

Based on the results, suspensions of the SDDs can be held for at least 4 hours prior to dosing with no crystallization and no significant change in performance expected.

f. Accelerated Stability Testing

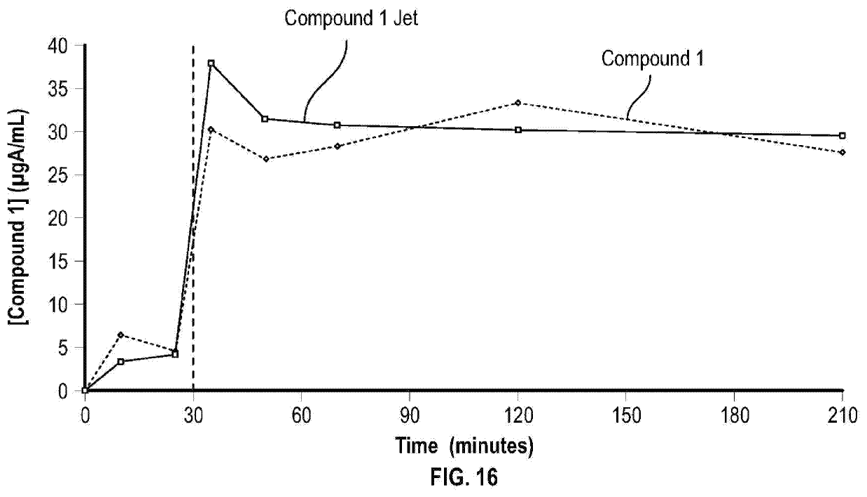

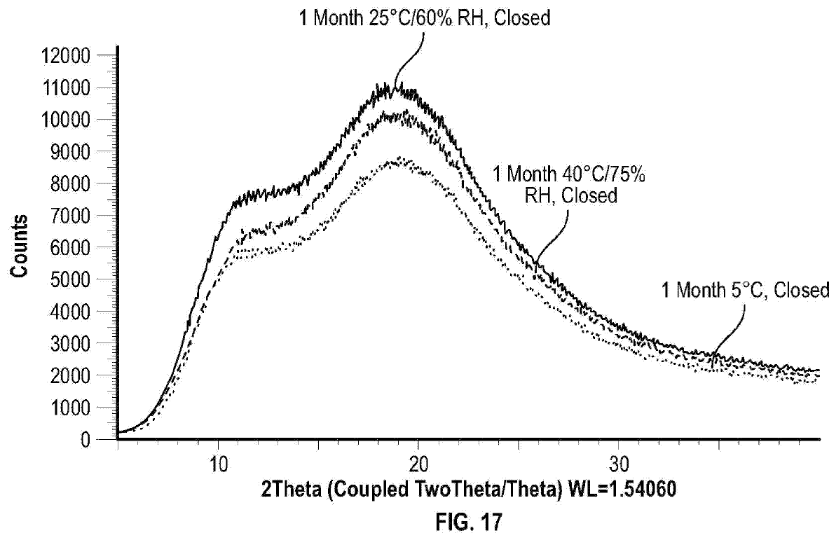

SDDs of Formulations 2 and 4 were aged for 4 weeks at 2-8° C., 25° C./60% RH, and 40° C./75% RH in closed packaging with a desiccant. The aged formulations were analyzed via PXRD. PXRD analysis of the aged SDD formulations shows that SDDs of Formulation 2 and 4 remain amorphous with no detectable crystalline material after 4 weeks. See FIGS. 17 and 18. FIG. 17 depicts the PXRD diffractograms of Formulation 2 after 4 weeks stability. FIG. 18 depicts the PXRD diffractograms of Formulation 4 after 4 weeks stability.

### Example 2: Micronization by Jet Milling

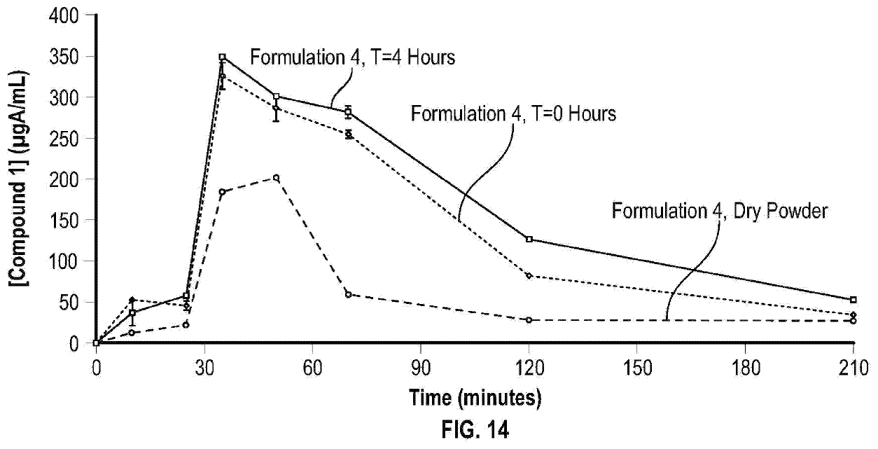

Compound 1 bulk drug material was subjected to particle size reduction through jet milling using a Jet-O-Mizer Mill. 224 mg of micronized Compound 1 was collected, resulting in an 22.4% recovery yield. From an SEM image of micronized Compound 1 at 10,000× magnification, the average particle size from visual observation was below 10 μm. X-ray diffraction of Compound 1 was performed to determine if any polymorphic conversion occurred during particle size reduction. Diffractorgrams of initial and milled Compound 1 indicated that the jet milling of the bulk material had no effect on crystalline form. FIG. 15 shows the PXRD diffractogram of micronized Compound 1 compared to bulk crystalline Compound 1.

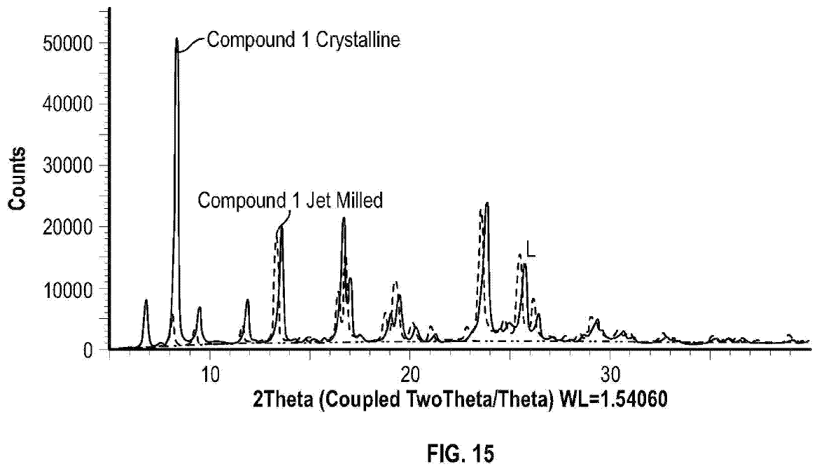

The dissolution performance of the jet-milled material and crystalline Compound 1 was tested in a non-sink dissolution test, to measure the supersaturation of the drug above the bulk crystalline Compound 1 solubility in biorelevant intestinal media (FaSSIF) after 30 minutes exposure to a low-pH environment (SGF). During the test, samples were transferred from SGF to FaSSIF SGF. FIG. 16 shows the SGF/FaSSIF non-sink dissolution test for micronized Compound 1 compared to bulk crystalline Compound 1. The results are reported below in Table 5.

TABLE 5

| | C_{max FaSSIF} | C₂₁₀ | AUC_{35-210 FaSSIF} |
|---|---|---|---|
| | (μgA/mL) | (μgA/mL) | (min * μgA/mL) |
| Compound 1-Jet Mill | 38.0 | 29.6 | 5400 |
| Crystalline Compound 1 | 33.4 | 27.6 | 5300 |

### Example 3: Tablet Formulations

An SDD of Formulation 4 was combined with excipients and incorporated into tablets.

In Formulation 10, detailed in Table 6, a SDD of Formulation 4 was combined with intragranular excipients and extragranular excipients. The intragranular excipients were microcrystalline cellulose, mannitol, talc, croscarmellose sodium and magnesium stearate. The extragranular excipients were microcrystalline cellulose, mannitol, talc, croscarmellose sodium, and magnesium stearate.

In Formulation 20, detailed in Table 7, a SDD of Formulation 4 was combined with intragranular excipients and extragranular excipients. The intragranular excipients were microcrystalline cellulose, sodium lauryl sulfate, mannitol, talc, croscarmellose sodium and magnesium stearate. The extragranular excipients were microcrystalline cellulose, mannitol, talc, croscarmellose sodium, sodium lauryl sulfate and magnesium stearate.

TABLE 6
Formulation 10

| Component | w/w % | mg/tablet |
|---|---|---|
| Intragranular | | |
| SDD of Formulation 4 | 50.0 | 500.0 |
| microcrystalline cellulose | 18.00 | 180.0 |
| Mannitol | 10.00 | 100.0 |
| Talc | 1.00 | 10.0 |
| croscarmellose sodium | 4.50 | 45.0 |
| magnesium stearate | 0.50 | 5.0 |
| Extragranular | | |
| microcrystalline cellulose | 4.00 | 40.0 |
| Mannitol | 7.75 | 77.5 |
| Talc | 1.00 | 10.0 |
| croscarmellose sodium | 3.00 | 30.0 |
| magnesium stearate | 0.25 | 2.5 |
| Total | 100.00 | 1000.0 |

TABLE 7
Formulation 20

| Component | w/w % | mg/tablet |
|---|---|---|
| Intragranular | | |
| SDD of Formulation 4 | 50.0 | 500.00 |
| microcrystalline cellulose | 17.75 | 177.5 |
| Mannitol | 9.75 | 97.5 |
| Talc | 1.00 | 10.0 |
| croscarmellose sodium | 4.5 | 45.0 |
| Sodium lauryl sulfate | 0.50 | 5.0 |
| magnesium stearate | 0.50 | 5.0 |
| Extragranular | | |
| microcrystalline cellulose | 4.00 | 40.0 |
| Mannitol | 7.25 | 72.5 |
| Talc | 1.00 | 10.0 |
| croscarmellose sodium | 3.00 | 30.0 |
| Sodium lauryl sulfate | 0.50 | 5.0 |
| magnesium stearate | 0.25 | 2.5 |
| Total | 100.00 | 1000.0 |

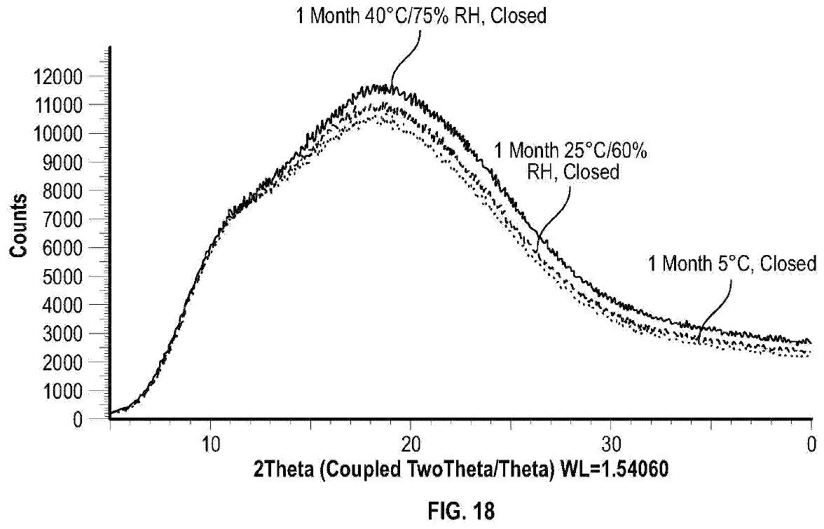

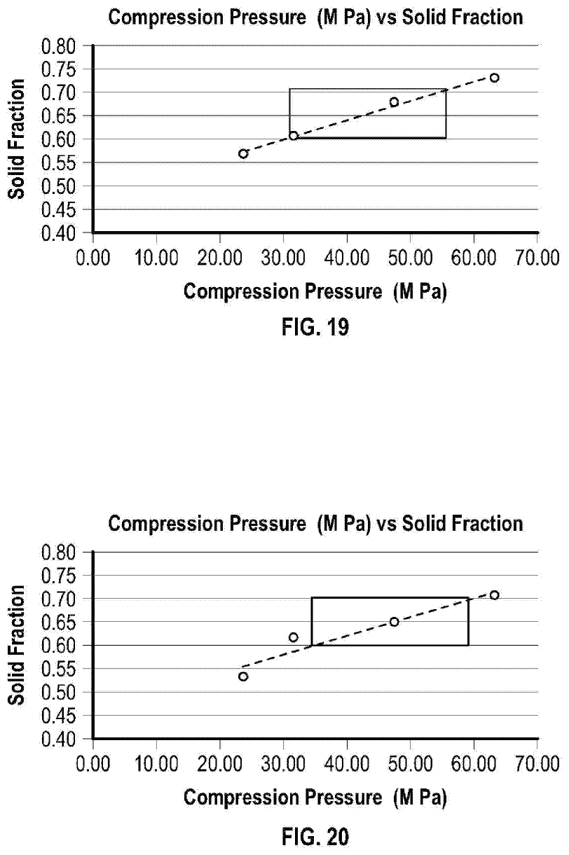

Tablets were prepared by granulation. FIGS. 19 and 20 depict the compression pressure (M Pa) vs. solid fraction for Formulations 10 and 20 into tablets. FIG. 19 presents the data for preparing tablets of Formulation 10 (without sodium lauryl sulfate), and FIG. 20 depicts the results for Formulation 20 (with sodium lauryl sulfate). The target solid fraction was 0.6-0.7.

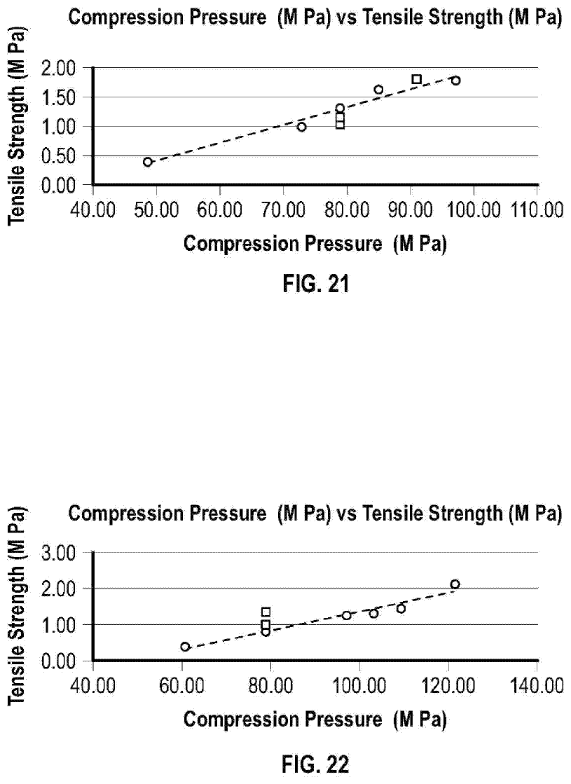

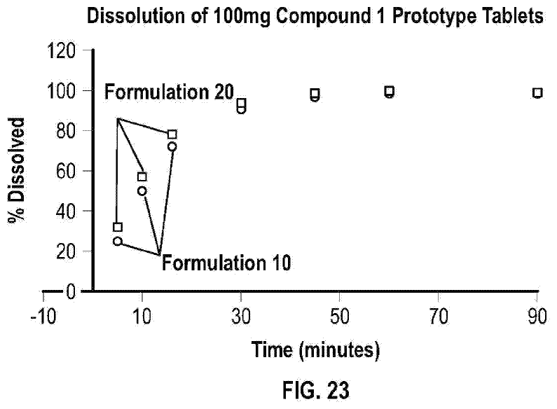

FIGS. 21 and 22 depict the compression pressure (M Pa) vs. tensile strength (M Pa) for Formulation 10 and Formulation 20. FIG. 21 depicts the compression pressure (M Pa) vs. tensile strength (M Pa) for Formulation 10 (without sodium lauryl sulfate), and FIG. 22 depicts compression pressure (M Pa) vs. tensile strength (M Pa) for Formulation 20 (with sodium lauryl sulfate). The tablet press used a Natoli Single Punch, with a 0.3750×0.7480″ Mod. Oval. The target tensile strength was 1.0-1.4 (M Pa).

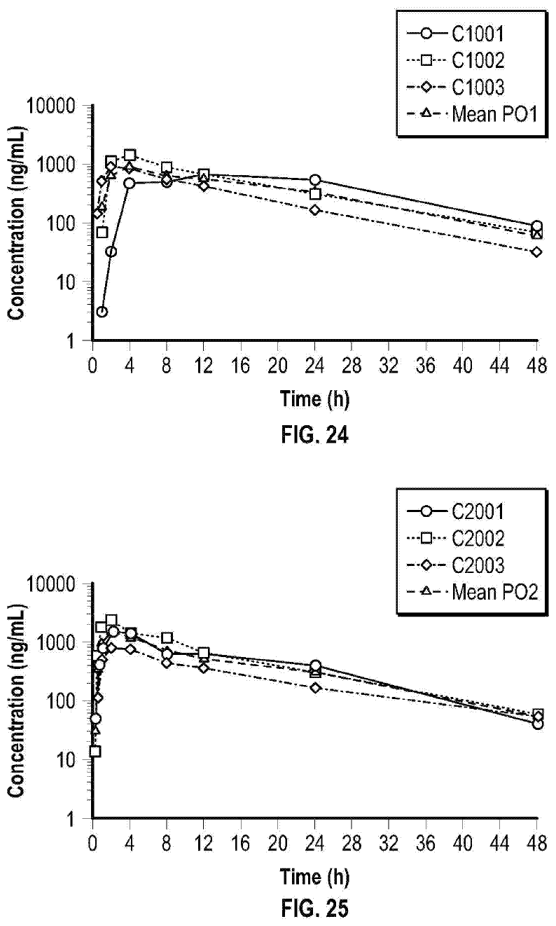

Tablets of Formulation 10 and Formulation 20 were investigated for dissolution. The medium used in the dissolution testing was 2.5% (w/v) CTAB (cetyl trimethylammonium bromide) in 0.01N HCl. Tablets dissolved to dose in approximately 45 minutes with the release profiles shown in FIG. 23.

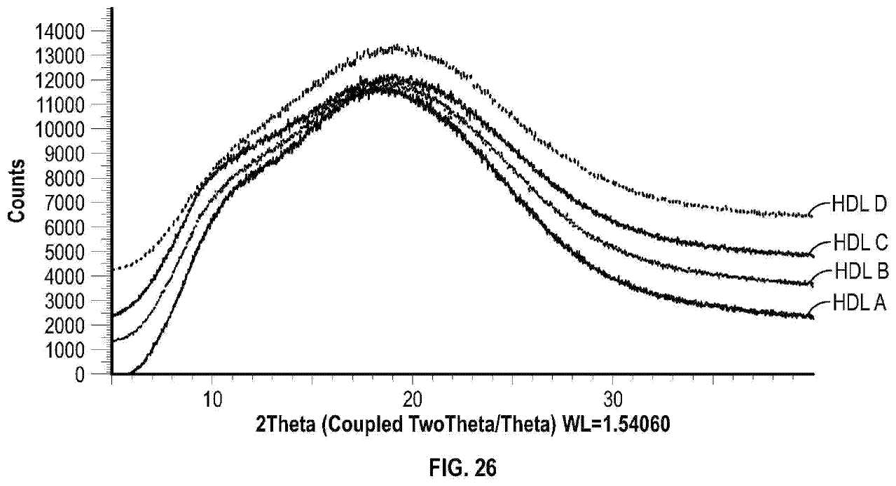

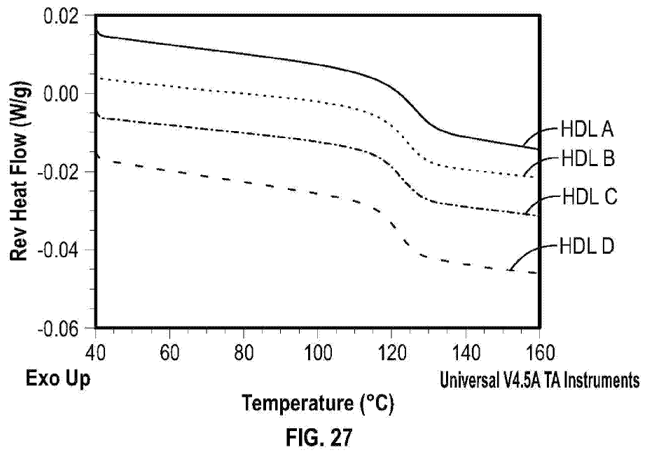

In vivo performance of each tablet (Formulation 10 and Formulation 20) (100 mg) was investigated in a monkey PK experiment. Results for Formulation 10 are shown in FIG. 24 (plasma concentration of Compound 1 after PO1 dosing at 100 mg/monkey) and results for Formulation 20 are shown in FIG. 25 (plasma concentration of Compound 1 after PO2 dosing at 100 mg/monkey). Formulation 20 had higher Cₘₐₓ and less Tₘₐₓ variability. Results are shown in Table 8.

TABLE 8
Formulation difference of systemic exposure to Compound 1 following single oral
dose of Formulation 10 tablet or Formulation 20 table to male cynomolgus monkeys.

| | Compared | Cₘₐₓ | | Tₘₐₓ | | | |
|---|---|---|---|---|---|---|---|
| | Doses | values | Cₘₐₓ | Values | Tₘₐₓ | AUC_{0-Last} | AUC_{0-Last} |
| Ratio | (mg/monkey) | (ng/mL | Ratio | (h) | Ratio | Values | Ratio |
| Formulation 20/ | 100 | 1570/993 | 1.58 | 2/6 | 0.333 | 18700/17000 | 1.10 |
| Formulation 10 | | | | | | | |

### Example 4: High Drug Loading Formulations

Spray dried solid dispersions comprising Compound 1 and hydroxypropyl methylcellulose phthalate (HPMCP HP-55) were investigated for higher drug loading. The SDDs were prepared as disclosed in Example 1, but with different amounts of Compound 1 and HPMCP HP-55 to result in the formulations reported in Table 9.

TABLE 9

| Formulation | Ratios |
|---|---|
| HDL A | 25:75 Compound 1:HPMCP HP-55* |
| HDL B | 33.3:67.7 Compound 1:HPMCP HP-55* |
| HDL C | 40:60 Compound 1:HPMCP HP-55* |
| HDL D | 50:50 Compound 1:HPMCP HP-55* |

*HPMCP HP-55 manufactured by Shin Etsu

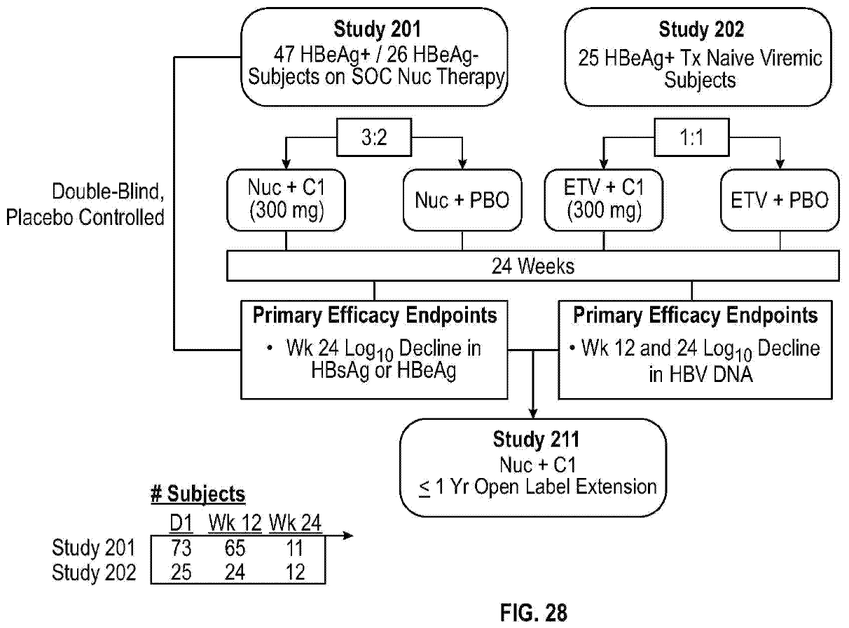

FIG. 26 depicts the PXRD results for the four high drug loading SDDs listed in Table 9. As shown in FIG. 26, the PXRD results for the four high drug loading SDDs indicate that all four of the high drug loaded SDDs are amorphous.

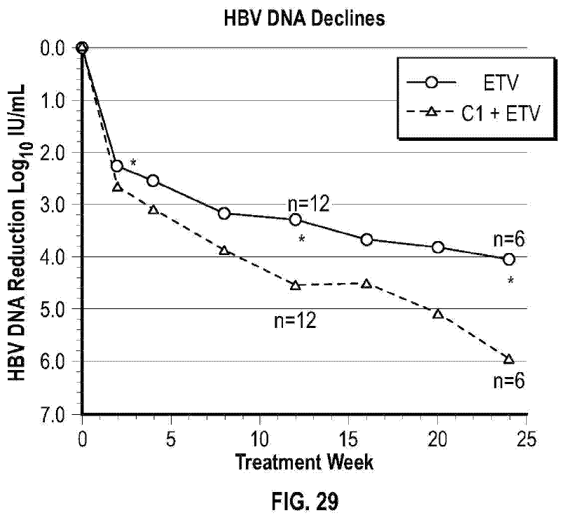

FIG. 27 depicts the MDSC results for the four high drug loading SDDs. As shown in FIG. 27, the high drug loaded SDDs show a single, high T_{g}. Stability for the formulations were investigated after one month. No change in the chemical profile was observed after one month. Thus, the four high-drug loaded SDDs were stable after one month.

### Example 5: Formulation

Spray dried solid dispersions comprising Compound 1 and hydroxypropyl methylcellulose phthalate (HPMCP HP-55) were as disclosed in Example 1, but with different amounts of Compound 1 and HPMCP HP-55 to result in the formulations reported in Table 10 and in Table 11.

TABLE 10

| | Composition (T1) | | Composition (T2) | | |
|---|---|---|---|---|---|
| Component | mg/Tablet | % | mg/Tablet | % | Function |

Intra-granulation (Dry granulation)

| | | | | | |
|---|---|---|---|---|---|
| 20:80 Compound | 500.0 | 50.00 | 500.0 | 50.00 | Active |
| 1:HPMCP HP-55 SDD | | | | | Ingredient |
| Microcrystalline Cellulose | 217.5 | 21.75 | 210.0 | 21.00 | Filler |
| (Avicel PH 105) | | | | | |
| Mannitol (Parteck M100) | 170.0 | 17.00 | 0 | 0 | Filler |
| Dibasic Calcium | 0 | 0 | 200.0 | 20.00 | Filler |
| phosphate | | | | | |
| Croscarmellose Sodium | 75.00 | 7.50 | 62.50 | 6.25 | Disintegrant |
| Sodium Lauryl Sulfate | 10.00 | 1.00 | 10.00 | 1.00 | Wetting agent |
| (Kolliphor SLS Fine) | | | | | |
| Talc | 20.00 | 2.00 | 0 | 0 | Glidant |
| Colloidal Silicon Dioxide | 0 | 0 | 10.00 | 1.00 | Glidant |
| Magnesium Stearate | 5.000 | 0.50 | 5.000 | 0.50 | Lubricant |

Extra-granulation (Final Blending)

| | | | | | |
|---|---|---|---|---|---|
| Magnesium Stearate | 2.500 | 0.25 | 2.500 | 0.25 | Lubricant |
| Total tablet weight (mg) | 1,000.0 | | 1,000.0 | | |

TABLE 11
T3 Formulation

| | Composition | | | |
|---|---|---|---|---|
| Component | mg/Tablet | % | Function | Compendial |

Intra-granulation (Dry granulation)

| | | | | |
|---|---|---|---|---|
| 20:80 ABI- | 500.0 | 66.67 | Active Ingredient | In-house |
| H0731:HPMCP SDD | | | | standard |
| MCC, Avicel PH 105 | 115.0 | 15.33 | Filler | NF/EP |
| Dibasic Calcium | 75.00 | 10.00 | Filler | USP/NF/EP |
| phosphate | | | | |
| Croscarmellose | 39.375 | 5.25 | Disintegrant | USP/NF/EP |
| Sodium | | | | |
| Sodium Lauryl Sulfate | 7.500 | 1.00 | Wetting agent | NF/EP |
| Colloidal Silicon | 7.500 | 1.00 | Glidant | USP/NF/EP |
| Dioxide | | | | |
| Magnesium Stearate | 3.750 | 0.50 | Lubricant | EP/NF |

Extra-granulation (Final Blending)

| | | | | |
|---|---|---|---|---|
| Magnesium Stearate | 1.875 | 0.25 | Lubricant | EP/NF |
| Core tablet weight | 750.0 | | | |
| (mg) | | | | |

The formulations disclosed in Tables 10 and 11 were used in the studies in the Examples below. In Study 201 both T1 and T2 were used. In Study 202, only T2 was used. In Study 211, both T2 and T3 were used.

### Example 6: A Study of Compound 1+Nucleos(t)Ide as Finite Treatment for Chronic Hepatitis B Subjects

Subjects undergoing combination therapy with 300 mg of Compound 1 and standard of care nucleos(t)ide (SOC NUC) are assessed for sustained virologic response (SVR), such as sustained clearance of serum HBV DNA, quantitative and qualitative reduction in the viral antigens hepatitis B e antigen (HBeAg), and hepatitis B surface antigen (HBsAg), as well as exploratory biomarkers such as reduction in circulating HBV RNA.

Subjects receive 300 mg QD of Compound 1 tablets orally. Subjects continue on their SOC NUC (ETV, TDF or TAF) tablet QD orally as per approved package insert.

Subjects who on Day 1 have a ‘complete response’ undergo a consolidation treatment period with Compound 1+standard of care nucleos(t)ide (SOC NUC) for 28 weeks, after which time they discontinue both their Compound 1 and SOC NUC. Subjects are intensively monitored for an additional 24 weeks of post-treatment follow-up to assess for a SVR. After post-treatment follow-up, subjects are monitored for an additional 24 months during a long-term, off-treatment follow-up period for a total of up to 36 months.

Subjects who at Week 24 have a ‘non-response’ after receiving combination treatment of Compound 1+SOC NUC are discontinued from the study at their Week 28 visit and are followed while continuing on SOC NUC therapy alone for 12 more weeks.

Subjects who have not met ‘complete response’ criteria by Week 48 of this study are considered ‘partial responders’. Subjects continue combination therapy until Week 52 and then stop therapy with Compound 1 at Week 52 and are followed while on their SOC NUC through Week 76.

Subjects who have met ‘complete response’ criteria by their Week 48 visit continue combination therapy until Week 52, after which they stop all HBV treatment (both Compound 1+SOC NUC) and are monitored for an additional 24 weeks post-treatment follow-up to assess SVR at Week 76. After post-treatment follow-up, subjects are monitored for an additional 18 months during the long-term, off-treatment, follow-up period for up to 36 months.

The primary outcomes are: (1) Number of subjects with sustained HBeAg loss (<0.11 PEI units/mL) in HBeAg positive subjects [Time Frame: Baseline to Week 24]; (2) Number of subjects with sustained viral suppression (below the limits of detection=20 IU/mL) [Time Frame: Baseline to Week 24]; and (3) Number of subjects with loss or stable reduction of HBsAg to ≤100 IU/mL [Time Frame: Baseline to Week 24]

The secondary outcomes include: (1) Number of subjects with adverse events, premature discontinuations, abnormal safety laboratory results, abnormal electrocardiogram (ECG), or abnormal vital signs [Time Frame: Up to maximum Week 52]; (2) Number of subjects with abnormal alanine aminotransferase (ALT) at Baseline who have normal ALT at end of treatment (EOT) and end of study (EOS) [Time Frame: Non Responders: Baseline to Wk 28 (EOT), Wk 40 (EOS); Early Complete Responders: Baseline to Wk 28 (EOT), Month 36 (EOS); Partial Responders: Baseline to Wk 52 (EOT), Wk 76 (EOS); Late Complete Responders: Baseline to Wk 52 (EOT), Month 36 (EOS)]; and (3) Number of subjects with suppression/loss of viral antigen/DNA on combination treatment whose viral antigens rebound off therapy [Time Frame: Up to 36 months following End of Treatment].

The eligibility criteria for subjects include adults ages 18 years to 71 years and all sexes.

The Inclusion Criteria for subjects in the study are as follows:
1. Willing and able to provide informed consent.
2. Previously enrolled on a study of Compound 1 and completed the treatment period, with demonstrated compliance in the opinion of the investigator.
3. Female subjects must agree to use an effective birth control method for the duration of the study and follow-up, or be surgically sterile for at least 6 months, or at least 2 years postmenopausal with serum follicle-stimulating hormone (FSH) levels consistent with a postmenopausal status. Effective birth control methods include male or female condom (may not be used together due to increased risk of breakage), vasectomy, intrauterine device (IUD), diaphragm, or cervical cap. Female subjects of childbearing potential must have a negative serum pregnancy test.
4. All heterosexually active male subjects must agree to use an effective birth control method for the duration of the study and follow-up. Effective birth control methods include male or female condom (may not be used together due to increased risk of breakage), vasectomy, hormone-based contraception (only female partner of a male subject), IUD, diaphragm, or cervical cap.
5. Agreement to adhere to Lifestyle Considerations (including abstaining from alcohol abuse [defined as alcohol consumption exceeding 2 standard drinks per day on average (1 standard drink=10 grams of alcohol)] and the use of illicit substances, herbal or other substances, or unnecessary over-the-counter medications throughout study duration.
6. In good general health except for chronic HBV infection.
7. Have the ability to take oral medication and be willing to adhere to the Compound 1 regimen.

The Exclusion Criteria for subjects in the study are as follows:
1. Must not have had evidence of HBV resistance-associated variants (RAVs) or lack of compliance on a previous study of Compound 1.
2. Must not have had a treatment-emergent adverse event or laboratory abnormalities deemed clinically significant and possibly or probably related to drug while on a previous study of Compound 1, that in the opinion of the Investigator or the Sponsor makes the subject unsuitable for this study.
3. Current clinically significant cardiac or pulmonary disease, chronic or recurrent renal or urinary tract disease, liver disease other than HBV, endocrine disorder, autoimmune disorder, diabetes mellitus requiring treatment with insulin or hypoglycemic agents, neuromuscular, musculoskeletal, or mucocutaneous conditions requiring frequent treatment, seizure disorders requiring treatment, or other medical conditions requiring frequent medical management or pharmacologic or surgical treatment that in the opinion of the Investigator or the Sponsor makes the subject unsuitable for the study.
4. Females who are lactating or pregnant or wish to become pregnant within the duration of the Compound 1 study.

### Example 7: A Study Evaluating Compound 1+NUC Vs NUC Alone for the Treatment of Viremic HBeAg-positive, CHB Subjects

Compound 1 is investigated in F0-F2 liver fibrosis (or equivalent) chronic hepatitis B subjects in a double blinded, placebo (Pbo) controlled study. In this study (also referred to as study 202), 25 treatment-naive HBeAg positive viremic subjects are randomized 1:1 (Group 1: Group 2) to Entecavir (ETV)+300 mg Compound 1 or ETV+Pbo treatment regimens. FIG. 1 depicts a flow chart of the study.

Subjects (Group 1) with chronic HBV who are currently not being treated receive Compound 1 along with SOC NUC (entecavir [ETV]) tablets orally for 24 weeks. Subjects receive 300 mg QD of Compound 1 tablets orally and SOC NUC (0.5 mg QD of ETV) orally as per approved package insert.

Subjects (Group 2) with chronic HBV who are currently not being treated receive matching placebo along with SOC NUC (entecavir [ETV]) tablets orally for 24 weeks. Subjects receive SOC NUC (0.5 mg QD of ETV) orally as per approved package insert and matching QD placebo tablets orally.

The primary outcome is a change in mean log₁₀ HBV DNA from Baseline (Day 1) to Week 12 or Week 24 on Compound 1+ETV as compared to placebo+ETV.

The secondary outcomes are as follows: (1) Number of subjects with adverse events, premature discontinuations, abnormal safety laboratory results, electrocardiogram (ECG), or vital signs [Time Frame: Up to Follow-up (maximum up to Week 36)]; (2) Number of subjects with abnormal alanine aminotransferase (ALT) at Baseline who have normal ALT at Week 24 on Compound 1+ETV as compared to placebo+ETV [Time Frame: Baseline to Week 24]; (3) Percentage of subjects with a decline in viral DNA to below limit of quantitation (LOQ; on Compound 1+ETV as compared to placebo+ETV) at end of treatment [Time Frame: Baseline, Weeks 2, 4, 8, 12, 16, 20, 24, 28, and 36]; Percentage of participants with HBV DNA levels below LLOQ are evaluated; (4) Median time to viral suppression, defined as HBV DNA <20 IU/mL, on Compound 1+ETV as compared to placebo+ETV [Time Frame: Baseline, Weeks 2, 4, 8, 12, 16, 20, 24, 28, and 36]; Median time to viral suppression are calculated and evaluated between subjects on Compound 1+ETV as compared to placebo+ETV; (5) Number of subjects with emergence of resistant HBV variants on Compound 1+ETV as compared to Placebo+ETV [Time Frame: Baseline to Week 36]; (6) Trough levels of Compound 1 on Compound 1+ETV therapy [Time Frame: Baseline, Weeks 2, 4, 12, 24, and 28]; Plasma concentrations of Compound 1 co-administered with SOC NUC (ETV) are determined; (7) Trough to peak ratios of Compound 1 on Compound 1+ETV therapy [Time Frame: Baseline, Weeks 2, 4, 12, 24, and 28]; Plasma concentrations of Compound 1 co-administered with SOC NUC (ETV) are determined; (8) Trough levels of ETV on Compound 1+ETV therapy as compared with placebo+ETV therapy [Time Frame: Baseline, Weeks 2, 4, 12, 24, and 28]; Plasma concentrations of SOC NUC (ETV) administered are determined; and (9) Trough to peak ratios of ETV on Compound 1+ETV therapy as compared with placebo+ETV therapy [Time Frame: Baseline, Weeks 2, 4, 12, 24, and 28]; Plasma concentrations of SOC NUC (ETV) administered are determined.

The Key Inclusion Criteria for subjects are as follows:
Male or female between ages 18 and 70 years
HBeAg-positive at screening
In good general health except for chronic HBV infection
HBV viral load ≥2×105 IU/mL
HBsAg >1000 IU/mL at screening

The Key Exclusion Criteria for subjects are as follows:
Any prior treatment with lamivudine or telbivudine, previous treatment with an investigational agent for HBV other than Compound 1; or any other SOC treatment for >4 weeks
Co-infection with HIV, HCV, HEV or HDV
History or evidence of hepatic decompensation (including gastrointestinal bleeding or esophageal varices) at any time prior to or at time of screening
Clinically significant cardiac or pulmonary disease, chronic or recurrent renal or urinary tract disease, liver disease other than HBV, endocrine disorder, autoimmune disorder, diabetes mellitus requiring treatment with insulin or hypoglycemic agents, neuromuscular, musculoskeletal, or mucocutaneous conditions requiring frequent treatment, seizure disorders requiring treatment, or other medical conditions requiring frequent medical management or pharmacologic or surgical treatment that in the opinion of the Investigator or the Sponsor makes the subject unsuitable for the study
Previous treatment with an investigational agent for HBV other than

Compound 1 in the last 6 months before screening
History of HCC
Females who are lactating or pregnant or wish to become pregnant are excluded from the study
Exclusionary laboratory parameters at screening:
   Platelet count <100,000/mm3
   Albumin <lower limit of normal (LLN)
   Direct bilirubin >1.2×ULN
   ALT >10×ULN at screening
   Serum alpha fetoprotein (AFP) ≥100 ng/mL. If AFP at Screening is >ULN but <100 ng/mL, subject is eligible if a hepatic imaging study prior to the initiation of study drug reveals no lesions suspicious of possible HCC
   International Normalized Ratio (INR) >1.5×ULN
   Glomerular filtration rate (GFR)<60 mL/min/1.73 m2 by CKD-EPI equation

Subjects return to the clinic at weeks (wk) 2 and 4 and then monthly up to wk 24. Clinical labs, safety and PK are monitored, as well as HBV biomarkers including HBV DNA, HBV RNA, HBsAg and HBeAg. At weeks 12 and 24, longitudinal serum samples are assayed for detectable virus. The Primary efficacy endpoints are log₁₀ decline in HBV DNA at wks 12/24, and the results are reported in Table 12 (below). Compound 1 is abbreviated as C1 in the tables and figures.

TABLE 12
Study 202 (Tx Naïve HBeAg+ subjects), mean log₁₀ declines

| Marker | Week | ETV (n) | | C1 + ETV (n) | | P values |
|---|---|---|---|---|---|---|
| RNA, copies/mL | 12 | 0.44 | (12) | 2.27 | (12) | <.005 |
| | 24 | 0.61 | (5) | 2.54 | (6) | <.005 |
| DNA, IU/mL | 12 | 3.29 | (12) | 4.54 | (12) | <.011 |
| | 24 | 3.99 | (6) | 5.94 | (6) | <.005 |

As shown in Table 12, subjects treated with Compound 1+ETV show a decrease in both viral RNA and viral DNA, and the combination of Compound 1+Nuc demonstrate superior antiviral activity vs. Nuc alone. FIG. 2 depicts the HBV DNA declines and FIG. 3 depicts the HBV RNA declines for the values reported in Table 12. As seen in FIG. 2 and FIG. 3, significantly faster and greater declines in HBV viremia (DNA/RNA) are seen in the combination therapy vs. Nuc alone.

### Example 8: A Study Evaluating Compound 1 as Adjunctive Therapy in Subjects with Chronic Hepatitis B

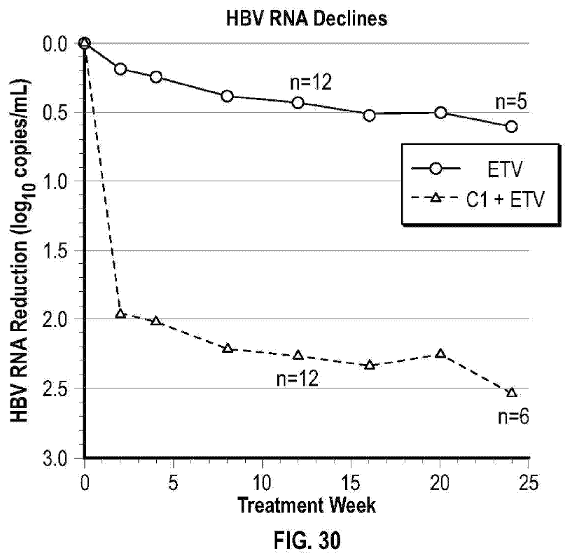

Compound 1 is investigated in F0-F2 liver fibrosis (or equivalent) chronic hepatitis B subjects in a double blinded, placebo (Pbo) controlled study. In this study (also referred to as study 201), 47 HBeAg positive and 26 HBeAg negative subjects already at viral suppression levels on standard of care (SOC) Nuc (ETV) are randomized 3:2 for the addition of Compound 1 (300 mg):Pbo to their SOC. FIG. 28 depicts a flow chart of the study.

Virologically suppressed subjects receive Compound 1 along with SOC NUC (ETV, TDF or TAF) tablets orally for 24 weeks. Subjects receive 300 mg QD Compound 1 tablets orally, and subjects continued on their SOC NUC (ETV, TDF or TAF) tablet orally (QD frequency) as per approved package insert.

Virologically suppressed subjects receive matching placebo tablets and continue their SOC NUC (ETV, TDF or TAF) for 24 weeks. Subjects receive matching QD placebo tablets orally, and subjects receive SOC NUC (ETV, TDF or TAF) tablet orally as per approved package insert.

The primary outcome is a change in mean log₁₀ serum viral antigen (HBsAg or HBeAg) from Baseline (Day 1) to Week 24 on Compound 1+SOC NUC as compared to placebo+SOC NUC [Time Frame: Baseline to Week 24]

The secondary outcomes are as follows: (1) Number of subjects with adverse events, premature discontinuations, abnormal safety laboratory results, electrocardiogram (ECG), or vital signs [Time Frame: Up to Follow-up (maximum up to Week 36)]; (2) Subjects with abnormal alanine aminotransferase (ALT) at Baseline who have normal ALT at Week 24 on Compound 1+NUC therapy as compared with placebo+NUC therapy [Time Frame: Baseline to Week 24]; (3) Trough levels of Compound 1 on Compound 1+SOC NUC therapy [Time Frame: Baseline, Weeks 2, 4, 12, 24, and 28]; (4) Trough to peak ratios of Compound 1 on Compound 1+SOC NUC therapy [Time Frame: Baseline, Weeks 2, 4, 12, 24, and 28]; (5) Trough levels of SOC NUC on Compound 1+SOC NUC therapy as compared with placebo+SOC NUC therapy [Time Frame: Baseline, Weeks 2, 4, 12, 24, and 28]; and (6) Trough to peak ratios of SOC NUC on Compound 1+SOC NUC therapy as compared with placebo+SOC NUC therapy [Time Frame: Baseline, Weeks 2, 4, 12, 24, and 28].

The Key Inclusion Criteria for the subjects in the study are as follows:
Male or female between ages 18 and 70 years
Virologically-suppressed (defined as HBV DNA ≤LOQ) for at least 6 months before screening on SOC NUC therapy
HBeAg-positive or HBeAg-negative at screening
In good general health except for chronic HBV infection

The Key Exclusion Criteria for the subjects in the study are as follows:
Co-infection with HIV, HCV, HEV or HDV
History or evidence of hepatic decompensation (including gastrointestinal bleeding or esophageal varices) at any time prior to or at time of screening
Clinically significant cardiac or pulmonary disease, chronic or recurrent renal or urinary tract disease, liver disease other than HBV, endocrine disorder, autoimmune disorder, diabetes mellitus requiring treatment with insulin or hypoglycemic agents, neuromuscular, musculoskeletal, or mucocutaneous conditions requiring frequent treatment, seizure disorders requiring treatment, or other medical conditions requiring frequent medical management or pharmacologic or surgical treatment that in the opinion of the Investigator or the Sponsor makes the subject unsuitable for the study
Previous treatment with an investigational agent for HBV other than Compound 1 in the last 6 months before screening
History of HCC
Females who are lactating or pregnant or wish to become pregnant are excluded from the study
Exclusionary laboratory parameters at screening include:
   Platelet count <100,000/mm3
   Albumin <lower limit of normal (LLN)
   Direct bilirubin >1.2×ULN
   ALT >5×ULN at screening
   International Normalized Ratio (INR) >1.5×ULN
   Glomerular filtration rate (GFR)<60 mL/min/1.73 m2 by CKD-EPI equation

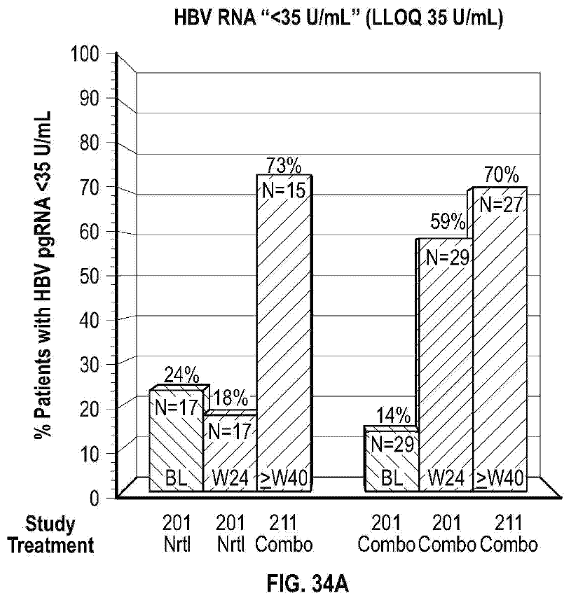

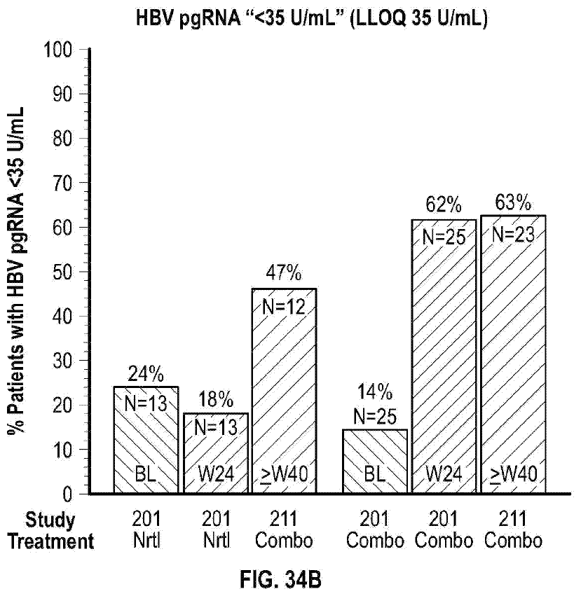

Subjects return to the clinic at weeks (wk) 2 and 4 and then monthly up to wk 24. Clinical labs, safety and PK are monitored, as well as HBV biomarkers including HBV DNA, HBV RNA, HBsAg and HBeAg. At weeks 12 and 24, longitudinal serum samples are assayed for detectable virus. FIG. 31 depicts the HBV DNA PCR Assay results at Week 24 for the Nuc monotherapy and FIG. 32 depicts the HBV DNA PCR Assay results at Week 24 for Compound 1+Nuc combo therapy. As shown in FIG. 31, residual viremia is not eliminated with the Nuc monotherapy. As shown in FIG. 32, residual viremia declined to below detection level (2-5 IU/mL). Thus, residual viremia is not eliminated by Nuc therapy, but by the combination therapy (Compound 1+Nuc). Additionally, as shown in Table 13, subjects on the combination therapy achieved rapid RNA declines. Of the subjects with detectable baseline RNA, 60% on the combination therapy achieved RNA<LOQ (200 copies/mL) by Week 16 vs. 0% on Nuc monotherapy.

TABLE 13

| | | | | |
|---|---|---|---|---|
| Marker | Week | Nuc (n) | C1 + Nuc (n) | P values |

Study 201 (Nuc-suppressed HBeAg+ subjects)

| | | | | | | |
|---|---|---|---|---|---|---|
| RNA, mean | 12 | 0.05 | (18) | 2.34 | (23) | <.001 |
| log₁₀ declines | 24 | 0.15 | (4) | 2.20 | (5) | 0.012 |

Study 201 (Available subjects at week 24), HBV DNA (+/−)

| | | | | | | |
|---|---|---|---|---|---|---|
| DNA, PCR TND* | 24 | 0 | (4) | 5 | (6) | N/A |

*TND = target not detected using ASMB <5 copies/mL semi-quantitative PCR assay

### Example 9

Compound 1 was investigated for potential inhibition of CYPs 2C19, 2D6, 2C8, 3A4 or 2B6, and induction of 3A4 or 2B6 in 58 healthy volunteers (HV) in three parts. HVs in part 1 received index substrates caffeine, tolbutamide, omeprazole, and dextromethorphan in a cocktail with and without Compound 1, and later received repaglinide with and without Compound 1. Part 2 HVs received Compound 1 at 300 mg QD on days 2 through 15. On days 1, 7 and 15, midazolam was co-administered with Compound 1 at 300 mg PO. Part 3 HVs received Compound 1 at 300 mg QD on days 11 through 30. On days 1, 16, and 26, bupropion was co-administered with Compound 1 at 300 mg.

In all studies, Compound 1 alone or in combination was well-tolerated. In studies 201 and 202, no clinically meaningful C_{trough} changes to ETV, TAD or TAF were found for each combination of Nuc plus Compound 1 at 300 mg. C_{trough} levels of Compound 1 300 mg were similar to monotherapy cohorts from previous studies of Compound 1. No clinically meaningful changes in AUC and Cₘₐₓ for the index substrates monitoring CYPs 2C9, 2C19, 2D6, 2C8, 3A4 or 2B6 were identified.

Data support long-term combination therapy dosing without alteration of either Compound 1 or Nuc dose regimens in the combinations studied. Co-administration studies with sensitive index substrates further indicate that Compound 1 has low potential for inhibition of CYPs 2C19, 2D6, 2C8, 3A4 or 2B6, and no induction of 3A4 or 2B6.

### Example 10

Compound 1 was investigated in an open-label, extension study (Study 211) to evaluate the safety and efficacy of combination therapy and Compound 1's effect on sustained viral response biomarkers. Of the 97 subjects completing Study 201 or Study 202, 87 received Compound 1 and Nrtl (nucleo(t)ide reverse tanscriptase inhibitors) and had been treated for at least 16 weeks in Study 211. Study 211 utilized the four assays described in Studies 201 and 202.

Participants with chronic HBV who were currently not being treated received Compound 1 along with SOC NUC (entecavir [ETV]) tablets orally for 24 weeks. Eligible participants entered a separate extension study after Week 24 to continue open-label Compound 1 for up to an additional year.

Switching from ETV to Compound 1+ETV resulted in immediate and enhanced declines in both HBV DNA and pgRNA levels, confirming the contribution of Compound 1 to the combination. The mean HBV DNA and pgRNA declines from baseline at Week 48 were 6.3 logs and 3.0 logs, respectfully, for patients with Compound 1 and ETV. Continued HBV DNA declines were observed on combination therapy. The observed acceleration in second phase decline of HBV pgRNA levels likely reflects reductions of cccDNA pools.

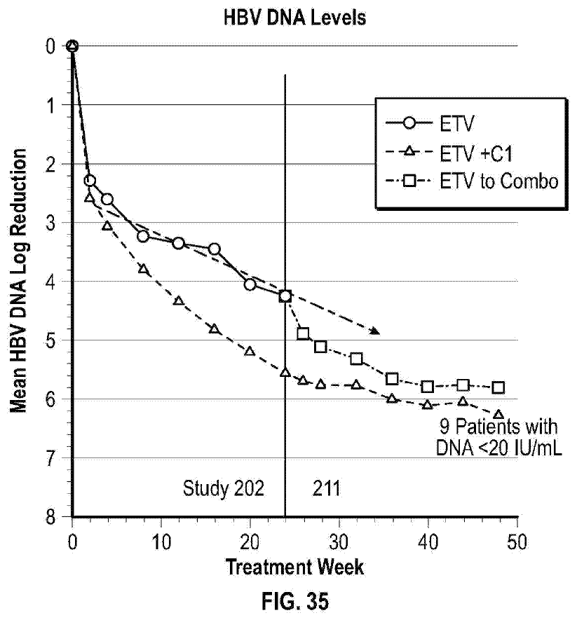

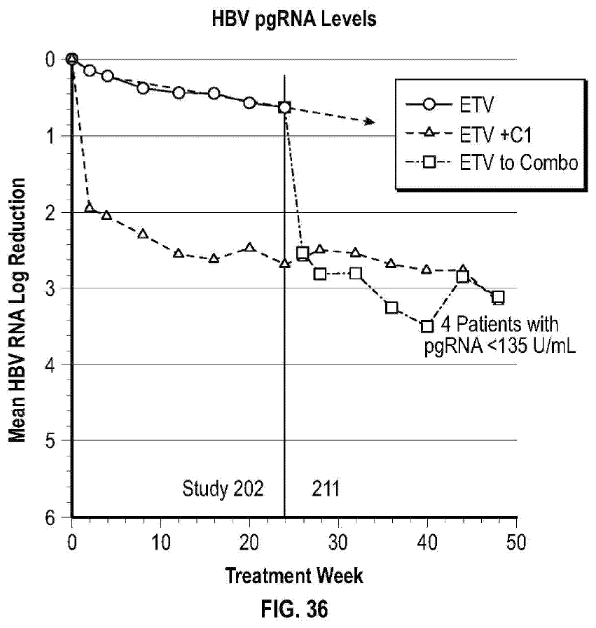

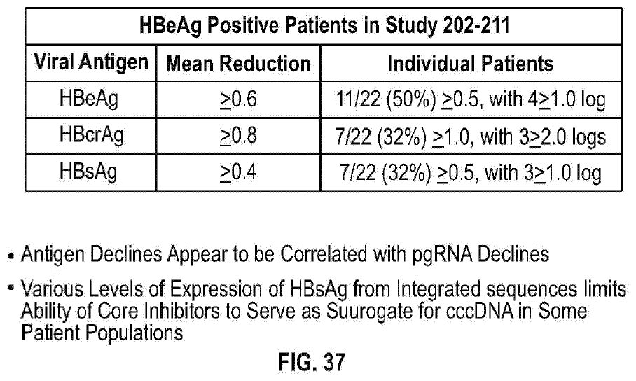

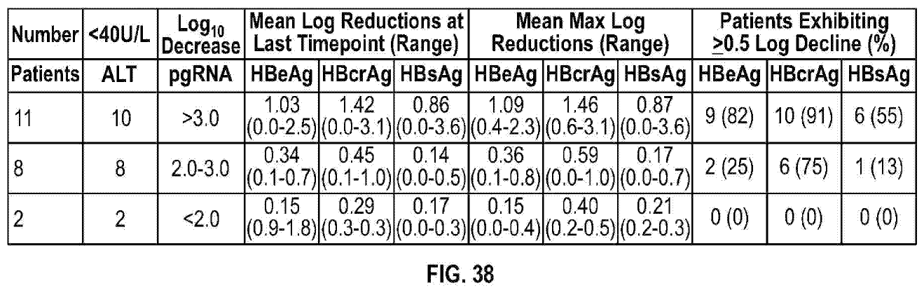

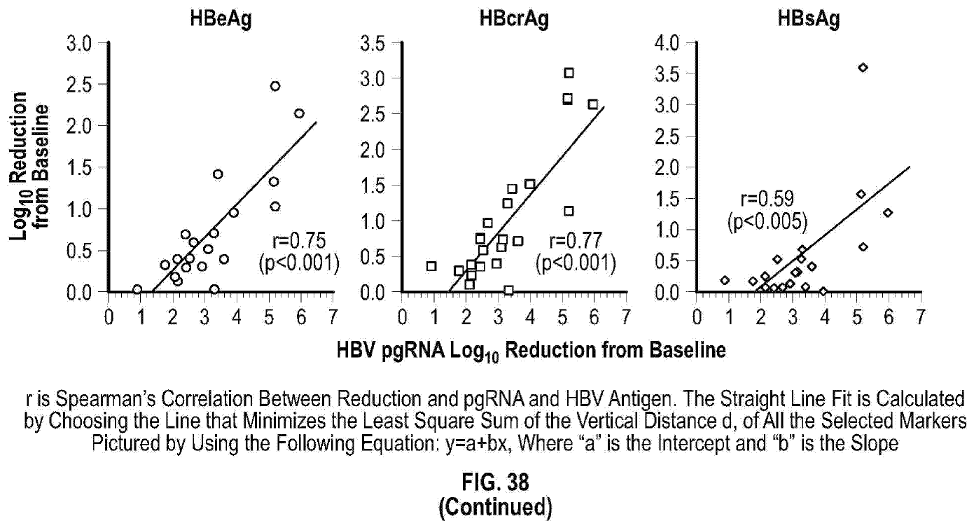

Only patients receiving Compound 1+ETV had reduced HBV DNA levels to TND and pgRNA levels to <35 U/mL. FIG. 33 shows the percentage of patients with HBV DNA in the open label with HBV DNA at undetectable limits. FIG. 34 shows the percentage of patients with HBV RNA in the open label with HBV RNA levels less than 35 U/mL. FIG. 35 shows the HBV DNA Log Reduction by treatment week. FIG. 36 shows the mean HBV RNA Log Reduction by treatment week. FIG. 37 summarizes the HBeAg reduction levels in patents.

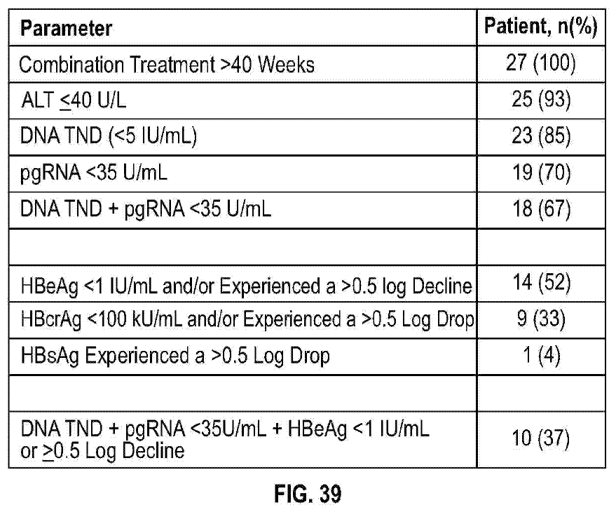

The addition of Compound 1 resulted in multi-log reduction in pgRNA levels while Nrtl therapy fails to significantly reduce pgRNA levels. FIG. 38 depicts the correlations between HBV pgRNA reductions and viral antigen declines (patients treated 16-60 weeks with Compound 1 and ETV in Study 202/211).

The initial phase decline of pgRNA (≤2 logs) was not associated with HBV antigen declines. The second phase of pgRNA appears to reflect decline in cccDNA pools, as pgRNA reductions greater than 3 logs are associated with the greatest level of declines in HB3Ag and HBcrAg (surrogate markers of cccDNA).

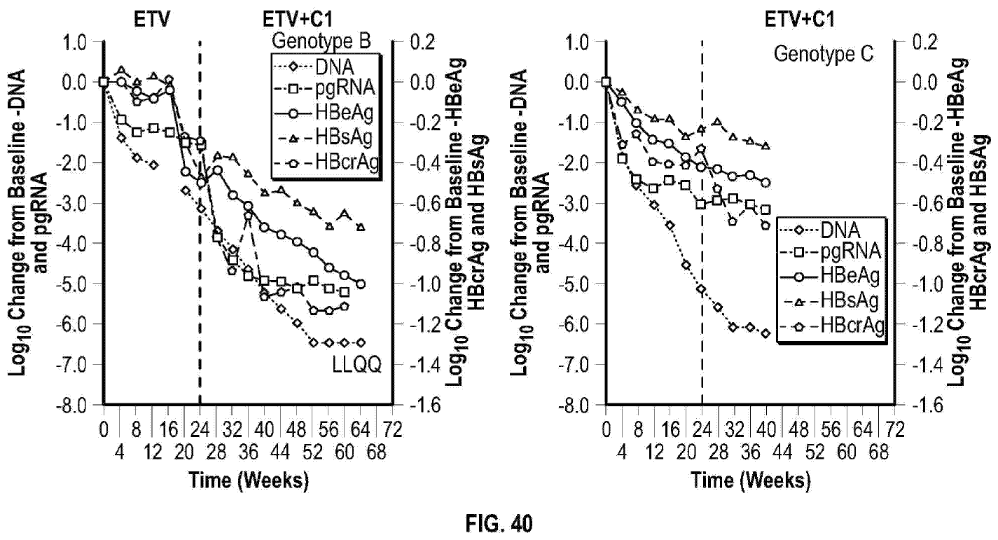

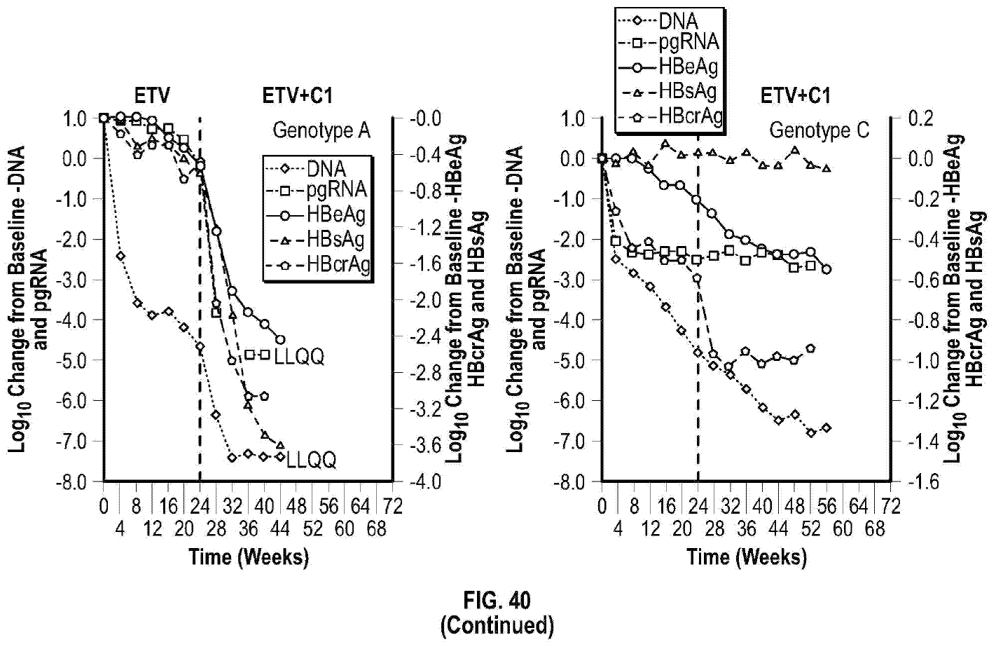

FIG. 39 summarizes the progression of viral markers in HBV Nrtl-Suppressed patients (patients treated 16-60 weeks with Compound 1 and Nrtl in Study 201/211). Viral markers in the patients received long term Nrtl treatment are significantly lower than in Rx-naïve patients, with several approaching the LLOQ. Results are supportive of mixed source (cccDNA and integrants) HBsAg in long term HBeAg-negative and Nrtl-suppressed patients that appear different than other viral antigents. FIG. 40 summarizes Study 202/211 individual patients.

The combination of Compound 1+Nrtl demonstrated faster and greater reductions in viral nucleic acid levels than Nrtl therapy alone with DNA TND and pgRNA <35 U/mL thresholds only being achieved in patients receiving Compound 1 and Nrtl.

Long term treatment with Compound 1+Nrtl results in continued deep reductions in HBV DNA and pgRNA as measured by high sensitivity PCR assays.

Second phase declines in pgRNA (>3 logs), a primary surrogate marker of cccDNA, were strongly associated with reductions in viral antigens, suggesting declining cccDNA pools.

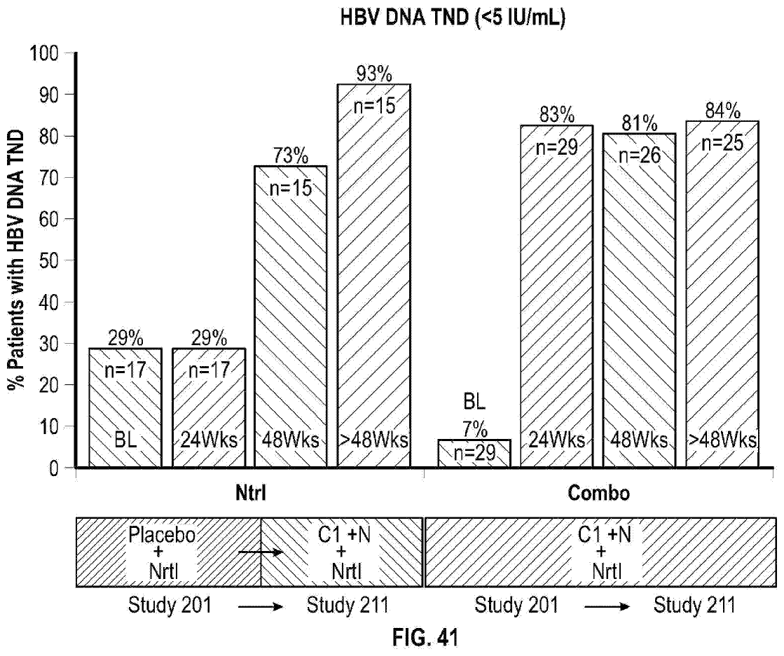

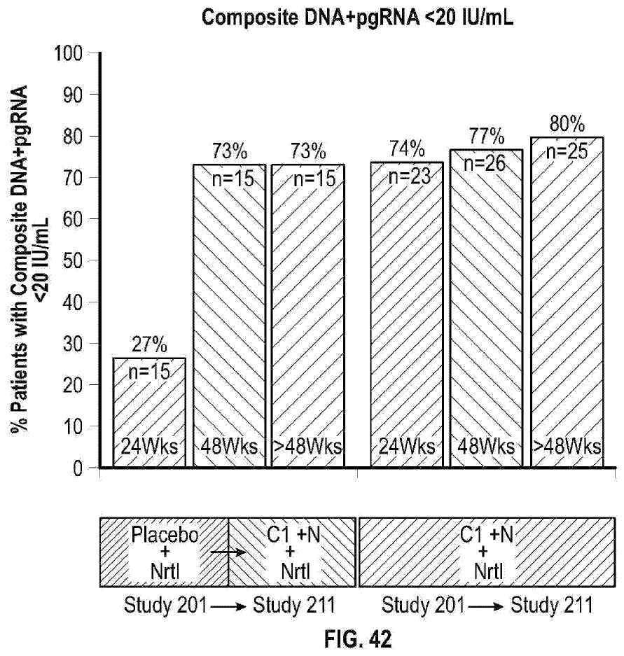

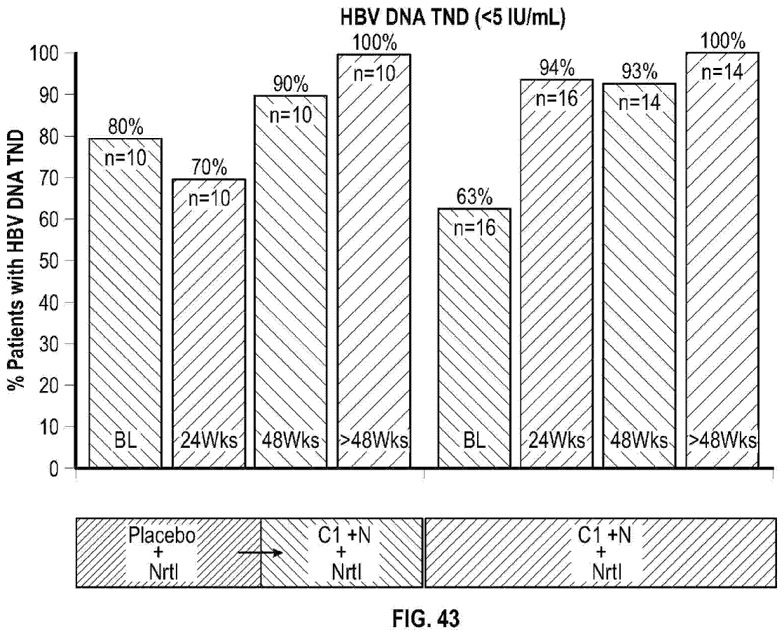

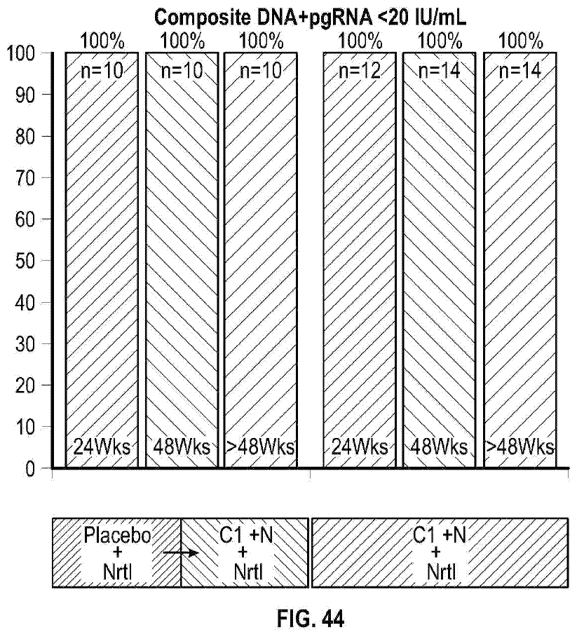

Patients in Studies 201, 202 and 211 were further monitored after 48 weeks of treatment. The results are provided in FIG. 40-FIG. 43. FIG. 40 depicts the log₁₀ change from baseline for patients in Study 202/211. FIG. 41 depicts the percentage of patients with HBV DNA TND for study 201/211. FIG. 42 depicts the percentage of patients with composite DNA and pgRNA less than 20 IU/mL. FIG. 43 depicts the percentage of patients with HBV DNA TND. FIG. 44 depicts the percentage of patients with DNA and pgRNA less than 20 IU/mL.

### Example 11: Compound 1 Combination Therapy Stopping Criteria

Compound 1 in combination with a nucleos(t)ide inhibitor is investigated for sustained virologic response (SVR). Subjects received 76 weeks of combination treatment (300 mg of Compound 1 and entecavir), as disclosed in Examples 7 and 8 herein. Other subjects received placebo and the entecavir from weeks 0 up to week 24, then received the combination of 300 mg of Compound 1 and entecavir for weeks 24-weeks 76. These subjects were also evaluated for meeting the stopping criteria at week 76.

In the study, virologically suppressed HBeAg negative patients, virologically suppressed HBeAg positive patients, and treatment naïve patients are assessed during the 76 week period for HBV nucleic acid concentration and HBeAg concentrations.

The stopping criteria is defined as a subject or patient having total HBV nucleic acids less than 20 IU/mL and HBeAg negative or have HBeAg concentrations of less than or equal to 5 IU/mL for at least six months prior to treatment week 76. The stopping criteria is applied to both classes of subjects: subjects that received the placebo and entecavir for weeks 0 to 24, and for subjects that received Compound 1 and entecavir starting at week 0.

If virologically suppressed HBeAg negative patients and virologically suppressed HBeAg positive patients meet the stopping criteria, administration of the combination treatment is stopped and the patient is monitored for up to three years. Virologically suppressed HBeAg positive patients not meeting the stopping criteria are no longer administered Compound 1, but are administer the nucleos(t)ide inhibitor.

If a treatment naïve and HBeAg positive patient, has an initial virologic response (defined as pgRNA decline greater than or equal to 2.5 log 10 U/mL from baseline), at around week 76, then the treatment of Compound 1 and the nucleos(t)ide inhibitor continues for up to an additional 48 weeks after the 76 week treatment week. If the treatment naïve and HBeAg positive patient does not meet the initial virologic response, then the patient is no longer administered Compound 1 but continues with the nucleos(t)ide inhibitor and is monitored monthly for 12 weeks.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A pharmaceutical composition comprising:
a solid dispersion, wherein the solid dispersion comprises:
a compound represented by: 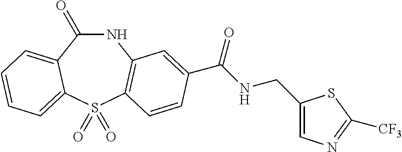 [SMILES: O=C(NCc1cnc(C(F)(F)F)s1)c1ccc2c(c1)NC(=O)c1ccccc1S2(=O)=O]
or a pharmaceutically acceptable salt thereof, and
a polymer;
wherein the solid dispersion comprises
about 10 wt % to about 50 wt % of the compound, or a pharmaceutically acceptable salt thereof, and
about 40 wt % to about 90 wt % of the polymer.

2. The pharmaceutical composition of claim 1, wherein the solid dispersion comprises about 15 wt % to about 30 wt % of compound, or a pharmaceutically acceptable salt thereof,
and
about 70 wt % to about 90 wt % of the polymer.

3. The pharmaceutical composition of claim 1, wherein the polymer is a methacrylate polymer or a cellulosic polymer.

4. The pharmaceutical composition of claim 1, wherein the polymer is selected from the group consisting of poly(methacrylic acid-co-methyl methacrylate), hypromellose acetate succinate, and hydroxypropyl methylcellulose phthalate.

5. The pharmaceutical composition of claim 1, wherein the solid dispersion is a spray-dried solid dispersion.

6. The pharmaceutical composition of claim 1, wherein the solid dispersion is a substantially amorphous solid dispersion.

7. The pharmaceutical composition of claim 1, wherein the solid dispersion is an amorphous solid dispersion.

8. The pharmaceutical composition of claim 7, wherein the solid dispersion has a single T_{g}.

9. The pharmaceutical composition of claim 8, wherein the solid dispersion is stable for at least four weeks.

10. The pharmaceutical composition of claim 1, wherein the pharmaceutical composition further comprises an excipient.

11. The pharmaceutical composition of claim 9, wherein the solid dispersion further comprises an excipient.

12. The pharmaceutical composition of claim 10, wherein the excipient is selected from the group consisting of a filler, sweetener, diluent, binder, lubricant, disintegrant, and glidant.

13. The pharmaceutical composition of claim 10, wherein the excipient is selected from the group consisting of microcrystalline cellulose, mannitol, talc, croscarmellose sodium, magnesium stearate, and sodium lauryl sulfate.

14. The pharmaceutical composition of claim 10, wherein the pharmaceutical composition further comprises a colorant, fragrance, or flavoring agent.

15. The pharmaceutical composition of claim 1, wherein the pharmaceutical composition is in a dose form selected from the group consisting of a granule, pellet, tablet, particle, and mini-tablet.

16. The pharmaceutical composition of claim 1, wherein the pharmaceutical composition comprises a pharmaceutically effective amount of the compound or a pharmaceutically acceptable salt thereof.

17. The pharmaceutical composition of claim 1, wherein the pharmaceutical composition is in a dose form comprising about 75 mg to about 125 mg of the compound, or a pharmaceutically acceptable salt thereof.

18. A method of treating Hepatitis B (HBV) in a patient in need thereof patient, comprising: administering to the patient a therapeutically effective amount of a pharmaceutical composition according to claim 1.

19. A method for preparing a pharmaceutical composition, the method comprising:
combining 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof, and
a polymer in a solvent thereby forming a mixture;
drying the mixture thereby forming a solid dispersion;
and
optionally combining the solid dispersion with an excipient.

20. The method of claim 19, wherein drying the mixture comprises spray drying the mixture.

21. The method of claim 19, wherein the solvent comprises water.

22. The method of claim 19, wherein the solvent comprises an organic solvent.

23. The method of claim 19, wherein the solvent comprises acetone and water.

24. The method of claim 19, wherein the polymer is selected from the group consisting of poly(methacrylic acid-co-methyl methacrylate), hypromellose acetate succinate, and hydroxypropyl methylcellulose phthalate.

25. The method of claim 19, wherein the solid dispersion is a substantially amorphous solid dispersion ion.

26. The method of claim 19, wherein the solid dispersion is an amorphous solid dispersion.

27. The method of claim 25, wherein the solid dispersion has a single T_{g}.

28. The method of claim 19, wherein the excipient is selected from the group consisting of a filler, sweetener, diluent, binder, lubricant, disintegrant, and glidant.

29. The method of claim 19, wherein the excipient is selected from the group consisting of microcrystalline cellulose, mannitol, talc, croscarmellose sodium, magnesium stearate, and sodium lauryl sulfate.

30. The method of claim 19, wherein the solid dispersion comprises
about 10 wt % to about 50 wt % of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof,
and
about 40 wt % to about 90 wt % of the polymer.

31. The method of claim 19, wherein the solid dispersion comprises
about 15 wt % to about 30 wt % of 11-oxo-N-((2-(trifluoromethyl)thiazol-5-yl)methyl)-10,11-dihydrodibenzo[b,f][1,4]thiazepine-8-carboxamide 5,5-dioxide, or a pharmaceutically acceptable salt thereof,
and
about 70 wt % to about 90 wt % of the polymer.

32. The method of claim 19, further comprising:
compressing the pharmaceutical composition into a tablet.

33. A method of treating hepatitis B in a subject in need thereof, the method comprising:
administering daily to the subject about 300 mg of a compound represented by: 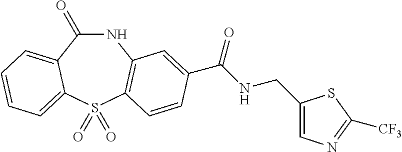 [SMILES: O=C(NCc1cnc(C(F)(F)F)s1)c1ccc2c(c1)NC(=O)c1ccccc1S2(=O)=O]
and
administering to the subject a therapeutically effective amount of a nucleos(t)ide inhibitor selected from the group consisting of entecavir, tenofovir, and tenofovir alafenamide fumarate.

34. The method of claim 33, wherein the subject is virologically suppressed and HBeAg negative before administering the compound.

35. The method of claim 33, wherein the subject is virologically suppressed and HBeAg positive before administering the compound.

36. The method of claim 33, wherein the subject is treatment naïve and HBeAg positive before administering the compound.

37. The method of claim 33, wherein the subject is virologically suppressed for at least 6 months before administration of the compound and has previously been administered a nucleos(t)ide inhibitor for the treatment of HBV.

38. The method of claim 33, wherein the subject has been previously administered a nucleos(t))ide inhibitor for the treatment of HBV for at least 2 months before administration of the compound.

39. The method of claim 33, wherein the subject has not been previously administered nucleos(t)ide inhibitor before administration of the compound.

40. The method of claim 33, wherein the subject has detectable levels of hepatitis B viral DNA prior to administration.

41. The method of claim 33, wherein the subject is HBeAg positive before administration of the compound.

42. The method of claim 37, wherein after 24 weeks of daily administration, the HBeAg positive subject has sustained HBeAg loss of <0.11 PEI units/mL.

43. The method of claim 37, wherein after 12 weeks of daily administration, the HBeAg positive subject has sustained HBeAg loss of <0.11 PEI units/mL.

44. The method of claim 37, wherein after 28 weeks of daily administration, the HBeAg positive subject has sustained HBeAg loss of <0.11 PEI units/mL.

45. The method of claim 37, wherein after 32 weeks of daily administration, the HBeAg positive subject has sustained HBeAg loss of <0.11 PEI units/mL.

46. The method of claim 37, wherein after 36 weeks of daily administration, the HBeAg positive subject has sustained HBeAg loss of <0.11 PEI units/mL.

47. The method of claim 37, wherein after 42 weeks of daily administration, the HBeAg positive subject has sustained HBeAg loss of <0.11 PEI units/mL.

48. The method of claim 37, wherein after 44 weeks of daily administration, the HBeAg positive subject has sustained HBeAg loss of <0.11 PEI units/mL.

49. The method of claim 33, wherein the subject is HBeAg negative before administration of the compound.

50. The method of claim 33, further comprising daily administration for at least 12 weeks, 24 weeks, 28 weeks, 32 weeks, 40 weeks, 44 weeks, 12 months, 18 months, 24 months, or 36 months.

51. The method of claim 33, wherein after 12 weeks, 24 weeks, 28 weeks, 32 weeks, 40 weeks, 44 weeks, 12 months, 18 months, 24 months, or 36 months of daily administration the subject has a reduction of HBeAg and/or HBsAg.

52. The method of claim 33, wherein after 12 weeks, 24 weeks, 28 weeks, 32 weeks, 40 weeks, 44 weeks, 12 months, 18 months, 24 months, or 36 months of daily administration the subject has a loss or stable reduction of HBsAg to ≤100 IU/mL.

53. The method of claim 33, wherein after 12 weeks, 24 weeks, 28 weeks, 32 weeks, 40 weeks, 44 weeks, 12 months, 18 months, 24 months, or 36 months of daily administration the subject has sustained viral suppression.

54. The method of claim 33, wherein after 12 weeks, 24 weeks, 28 weeks, 32 weeks, 40 weeks, 44 weeks, 12 months, 18 months, 24 months, or 36 months of daily administration the subject has a reduction in HBV DNA or HBV RNA.

55. The method of claim 54, wherein the HBV DNA reduction is below the detectable limit using a PCR-assay.

56. The method of claim 54, wherein the HBV RNA is below the limit of detection.

57. The method of claim 1, after 12 weeks, 24 weeks, 28 weeks, 32 weeks, 40 weeks, 44 weeks, 12 months, 18 months, 24 months, or 36 months of daily administration the subject has greater than 0.5 log₁₀ decline in HBeAg.

58. The method of claim 33, wherein the method reduces hepatitis B virus to below detection levels in the subject.

59. The method of claim 33, wherein the compound is in a solid dosage form.

60. The method of claim 33, wherein the compound is in a solid dispersion.

61. The method of claim 33, wherein the solid dispersion further comprises a polymer.

62. The method of claim 33, wherein the solid dispersion further comprises an excipient.

63.-88. (canceled)
